(19) United States
(12) Patent Application Publication
(10) Pub. No.: US 2023/0220106 A1
(43) Pub. Date: Jul. 13, 2023

(54) ANTIBODIES TARGETING 5T4 AND USES THEREOF

(71) Applicant: Dragonfly Therapeutics, Inc., Waltham, MA (US)

(72) Inventors: Pyae P. Hein, Malden, MA (US); Zong Sean Juo, Taipei (TW); Xinbi Li, Waltham, MA (US); Christopher Ryan Morgan, Southborough, MA (US)

(21) Appl. No.: 18/062,433

(22) Filed: Dec. 6, 2022

Related U.S. Application Data
(60) Provisional application No. 63/375,823, filed on Sep. 15, 2022.
(60) Provisional application No. 63/287,511, filed on Dec. 8, 2021.

(51) Int. Cl. C07K 16/30; C07K 16/28; A61P 35/00

(52) U.S. Cl. CPC C07K 16/30; A61P 35/00; C07K 16/2809; C07K 2317/24; C07K 2317/31; C07K 2317/92; C07K 2317/565; C07K 2317/622

(57) ABSTRACT

Disclosed are proteins with antibody heavy chain and light chain variable domains that can be paired to form an antigen-binding site targeting 5T4 on a cell, pharmaceutical compositions comprising such proteins, and therapeutic methods using such proteins and pharmaceutical compositions, including for the treatment of cancer.

### FIELD OF THE INVENTION

The disclosure provides proteins with antibody heavy chain and light chain variable domains that can be paired to form an antigen-binding site targeting 5T4 on a cell, pharmaceutical compositions comprising such proteins, and therapeutic methods using such proteins and pharmaceutical compositions, including for the treatment of cancer.

### CROSS-REFERENCE TO RELATED APPLICATIONS

This application claims the benefit under 35 U.S.C. § 119(e) of U.S. Provisional Application No. 63/287,511, filed on Dec. 8, 2021, and U.S. Provisional Application No. 63/375,823, filed on Sep. 15, 2022, which are hereby incorporated herein by reference in their entireties for all purposes.

### SEQUENCE LISTING

The instant application contains a Sequence Listing which has been submitted electronically in .XML file format and is hereby incorporated by reference in its entirety. Said .XML copy, created on created on Nov. 22, 2022, is named DFY-123-US-NP_SL.xml and is 268,288 bytes in size.

### BACKGROUND

Cancer continues to be a significant health problem despite the substantial research efforts and scientific advances reported in the literature for treating this disease. Some of the most frequently diagnosed cancers in adults include breast cancer and lung cancer. Current treatment options for these cancers are not effective for all patients and/or can have substantial adverse side effects. Other types of cancer also remain challenging to treat using existing therapeutic options. Slower replicating, stem-like cells of the tumor (i.e., cancer stem cells), may be causes of clinical relapse or recurrences after traditional therapies that target the rapidly proliferating cells that comprise the bulk of the tumor. Additionally, the tumor microenvironment, including cancer-associated fibroblasts (CAPs), often promotes malignancy and inhibits cancer therapies.

The human trophoblast glycoprotein 5T4 is an N-glycosylated transmembrane protein. Its expression is mechanistically associated with the directional movement of cells through epithelial mesenchymal transition, facilitation of CXCL12/CXCR4 chemotaxis, and blocking of canonical Wnt/beta-catenin while favoring non-canonical pathway signaling. These processes are highly regulated in development and in adult tissues, but they help drive the spread of cancer cells. It has been shown that 5T4 has very limited expression in normal adult tissue, but is widespread in many cancers including colorectal cancer, ovarian cancer, non-small cell lung cancer, renal cancer, breast cancer (e.g., hormone receptor positive (HR+) breast cancer), endometrial cancer, squamous cell carcinoma, head and neck squamous cell carcinoma, uterine cancer, pancreatic cancer, mesothelioma, and gastric cancer. Additionally, 5T4 has been linked to cancer stem cells (Harper J et al. Mol Cancer Ther. 2017). 5T4 may also be associated with the tumor microenvironment.

Accordingly, there remains a need in the field for new and useful antibodies that bind 5T4.

### SUMMARY OF THE INVENTION

The present disclosure provides antigen-binding sites or antigen-binding domains that bind 5T4. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating 5T4-associated diseases such as cancer.

Accordingly, in one aspect, the present disclosure provides an antigen-binding site or antigen-binding domain that binds 5T4, comprising: a heavy chain variable domain (VH) comprising a complementarity-determining region 1 (CDR1) sequence comprising SEQ ID NO:3, a complementarity-determining region 2 (CDR2) sequence comprising SEQ ID NO:4, and a complementarity-determining region 3 (CDR3) sequence comprising SEQ ID NO:5; and a light chain variable domain (VL) comprising a CDR1 sequence comprising SEQ ID NO:6, a CDR2 sequence comprising SEQ ID NO:7, and a CDR3 sequence comprising SEQ ID NO:8. In another aspect, provided is an antigen-binding site or antigen-binding domain that binds 5T4, comprising: a heavy chain variable domain (VH) comprising a CDR1 sequence comprising SEQ ID NO: 170, a CDR2 sequence comprising SEQ ID NO:172, and a CDR3 sequence comprising SEQ ID NO:5; and a light chain variable domain (VL) comprising a CDR1 sequence comprising SEQ ID NO:6, a CDR2 sequence comprising SEQ ID NO:7, and a CDR3 sequence comprising SEQ ID NO:8, wherein the complementarity-determining regions (CDRs) are according to Kabat. In another aspect, provided is an antigen-binding site or antigen-binding domain that binds 5T4, comprising: a heavy chain variable domain (VH) comprising a CDR1 sequence comprising SEQ ID NO:3, a CDR2 sequence comprising SEQ ID NO:182, and a CDR3 sequence comprising SEQ ID NO:183; and a light chain variable domain (VL) comprising a CDR1 sequence comprising SEQ ID NO:184, a CDR2 sequence comprising SEQ ID NO:185, and a CDR3 sequence comprising SEQ ID NO:186, wherein the CDRs are according to Chothia.

In another aspect, provided is an antigen-binding site or antigen-binding domain that binds 5T4, comprising: a heavy chain variable domain (VH) comprising a CDR1 sequence comprising SEQ ID NO:199, a CDR2 sequence comprising SEQ ID NO:200, and a CDR3 sequence comprising SEQ ID NO:201; and a light chain variable domain (VL) comprising a CDR1 sequence comprising SEQ ID NO:202, a CDR2 sequence comprising SEQ ID NO:185, and a CDR3 sequence comprising SEQ ID NO:8, wherein the CDRs are according to IMGT. In another aspect, provided is an antigen-binding site or antigen-binding domain that binds 5T4, comprising: a heavy chain variable domain (VH) comprising a CDR1 sequence comprising SEQ ID NO:216, a CDR2 sequence comprising SEQ ID NO:217, and a CDR3 sequence comprising SEQ ID NO:218; and a light chain variable domain (VL) comprising a CDR1 sequence comprising SEQ ID NO:219, a CDR2 sequence comprising SEQ ID NO:220, and a CDR3 sequence comprising SEQ ID NO:186, wherein the CDRs are according to Honegger. In some embodiments, the VH comprises an amino acid sequence of SEQ ID NO:9 and the VL comprises an amino acid sequence of SEQ ID NO:10. In some embodiments, the VH comprises an amino acid sequence of SEQ ID NO:11 and the VL comprises an amino acid sequence of SEQ ID NO:12. In some embodiments, the VH comprises an amino acid sequence of SEQ ID NO:22 and the VL comprises an amino acid sequence of SEQ ID NO:10. In some embodiments, the VH comprises an amino acid sequence of SEQ ID NO:24 and the VL comprises an amino acid sequence of SEQ ID NO:10. In some embodiments, the VH comprises an amino acid sequence of SEQ ID NO:108 and the VL comprises an amino acid sequence of SEQ ID NO:10. In some embodiments, the VH comprises an amino acid sequence of SEQ ID NO:138 and the VL comprises an amino acid sequence of SEQ ID NO:10. In some embodiments, the VH comprises an amino acid sequence of SEQ ID NO:26 and the VL comprises an amino acid sequence of SEQ ID NO:10. In some embodiments, the VH comprises an amino acid sequence of SEQ ID NO:28 and the VL comprises an amino acid sequence of SEQ ID NO:10. In some embodiments, the VH comprises an amino acid sequence of SEQ ID NO:30 and the VL comprises an amino acid sequence of SEQ ID NO:10. In some embodiments, the VH comprises an amino acid sequence of SEQ ID NO:1 and the VL comprises an amino acid sequence of SEQ ID NO:1.

In some embodiments, the VH comprises an amino acid sequence at least 90%, at least 91%, at least 92%, at least 93%, at least 94%, at least 95%, at least 96%, at least 97%, at least 98%, or at least 99% identical to the amino acid sequence of SEQ ID NO:9. In some embodiments, the VH comprises a G44C substitution relative to SEQ ID NO:9, wherein the numbering is according to Kabat. In some embodiments, the VH comprises the amino acid sequence of SEQ ID NO:11. In some embodiments, the VH comprises the amino acid sequence of SEQ ID NO:9.

In some embodiments, the VL comprises an amino acid sequence at least 90%, at least 91%, at least 92%, at least 93%, at least 94%, at least 95%, at least 96%, at least 97%, at least 98%, or at least 99% identical to the amino acid sequence of SEQ ID NO:10. In some embodiments, the VL comprises a G100C substitution relative to SEQ ID NO:10, wherein the numbering is according to Kabat. In some embodiments, wherein the VL comprises the amino acid sequence of SEQ ID NO:12. In some embodiments, the VL comprises the amino acid sequence of SEQ ID NO:10.

In another aspect, the present disclosure provides an antigen-binding site comprising a VH comprising the amino acid sequence of SEQ ID NO:94 and a VL comprising the amino acid sequence of SEQ ID NO:10.

In another aspect, the present disclosure provides an antigen-binding site or antigen-binding domain comprising a VH comprising at least 95% identity to the amino acid sequence of SEQ ID NO:9 and a VL comprising at least 95% identity to the amino acid sequence of SEQ ID NO:10, or a VH comprising at least 95% identity to the amino acid sequence of SEQ ID NO:11 and a VL comprising at least 95% identity to the amino acid sequence of SEQ ID NO:12. In some embodiments, the antigen-binding site or antigen-binding domain comprises a VH comprising at least 96% identity to the amino acid sequence of SEQ ID NO:9 and a VL comprising at least 96% identity to the amino acid sequence of SEQ ID NO:10, or a VH comprising at least 96% identity to the amino acid sequence of SEQ ID NO:11 and a VL comprising at least 96% identity to the amino acid sequence of SEQ ID NO:12. In some embodiments, the antigen-binding site or antigen-binding domain comprises a VH comprising at least 96% identity to the amino acid sequence of SEQ ID NO:9 and a VL comprising at least 96% identity to the amino acid sequence of SEQ ID NO:10, or a VH comprising at least 96% identity to the amino acid sequence of SEQ ID NO:11 and a VL comprising at least 96% identity to the amino acid sequence of SEQ ID NO:12. In some embodiments, the antigen-binding site or antigen-binding domain comprises a VH comprising at least 97% identity to the amino acid sequence of SEQ ID NO:9 and a VL comprising at least 97% identity to the amino acid sequence of SEQ ID NO:10, or a VH comprising at least 97% identity to the amino acid sequence of SEQ ID NO:11 and a VL comprising at least 97% identity to the amino acid sequence of SEQ ID NO:12. In some embodiments, the antigen-binding site or antigen-binding domain comprises a VH comprising at least 98% identity to the amino acid sequence of SEQ ID NO:9 and a VL comprising at least 98% identity to the amino acid sequence of SEQ ID NO:10, or a VH comprising at least 98% identity to the amino acid sequence of SEQ ID NO:11 and a VL comprising at least 98% identity to the amino acid sequence of SEQ ID NO:12. In some embodiments, the antigen-binding site or antigen-binding domain comprises a VH comprising at least 99% identity to the amino acid sequence of SEQ ID NO:9 and a VL comprising at least 99% identity to the amino acid sequence of SEQ ID NO:10, or a VH comprising at least 99% identity to the amino acid sequence of SEQ ID NO:11 and a VL comprising at least 99% identity to the amino acid sequence of SEQ ID NO:12.

In another aspect, the present disclosure provides an antigen-binding site or antigen-binding domain comprising a VH comprising the amino acid sequence of SEQ ID NO:9 and a VL comprising the amino acid sequence of SEQ ID NO:10, or a VH comprising the amino acid sequence of SEQ ID NO: 11 and a VL comprising the amino acid sequence of SEQ ID NO:12.

In another aspect, the present disclosure provides an antigen-binding site comprising a VH comprising the amino acid sequence of SEQ ID NO:9 and a VL comprising the amino acid sequence of SEQ ID NO:10.

In another aspect, the present disclosure provides an antigen-binding site comprising VH comprising the amino acid sequence of SEQ ID NO: 11 and a VL comprising the amino acid sequence of SEQ ID NO:12.

In another aspect, the present disclosure provides an antigen-binding site that binds 5T4, comprising: a VH comprising a CDR1 sequence comprising SEQ ID NO:47, a CDR2 sequence comprising SEQ ID NO:4, and a CDR3 sequence comprising SEQ ID NO:48; and a VL comprising a CDR1 sequence comprising SEQ ID NO:49, a CDR2 sequence comprising SEQ ID NO:7, and a CDR3 sequence comprising SEQ ID NO:8.

In another aspect, the present disclosure provides an antigen-binding site that binds 5T4 , comprising: (a) a VH comprising a CDR1 sequence comprising SEQ ID NO:53, a CDR2 sequence comprising SEQ ID NO:54, and a CDR3 sequence comprising SEQ ID NO:55; and a VL comprising a CDR1 sequence comprising SEQ ID NO: 56, a CDR2 sequence comprising SEQ ID NO: 57, and a CDR3 sequence comprising SEQ ID NO:8, or (b) a VH comprising a CDR1, a CDR2, and a CDR3 sequence from Table 5; and a VL comprising a CDR1 sequence comprising SEQ ID NO: 56, a CDR2 sequence comprising SEQ ID NO: 57, and a CDR3 sequence comprising SEQ ID NO:8.

In another aspect, the present disclosure provides an antigen-binding site that competes with the antigen-binding site provided herein.

In some embodiments, the antigen-binding site is present as a single-chain fragment variable (scFv), a Fab fragment, or a monoclonal antibody. In some embodiments, the antigen-binding site is present as a single-chain fragment variable (scFv). In some embodiments, the scFv comprises a sequence selected from the group consisting of SEQ ID NO:95 and SEQ ID NO:96. In some embodiments, the scFv comprises a sequence selected from the group consisting of SEQ ID NO: 13 and SEQ ID NO: 14.

In some embodiments, the antigen-binding site binds 5T4 within the LRR1 domain.

In some embodiments, an antigen-binding site of the present disclosure comprises a VH comprising a CDR1, a CDR2, and a CDR3 sequence from Table 1; and a VL comprising a CDR1, a CDR2, and a CDR3 sequence from Table 1. In some embodiments, the antigen-binding site comprises a VH and corresponding VL from Table 1.

In another aspect, the present disclosure provides a protein comprising the antigen-binding site provided herein. In some embodiments, the protein further comprises an antibody heavy chain constant region. In some embodiments, the antibody heavy chain constant region is a human IgG heavy chain constant region. In some embodiments, the antibody heavy chain constant region is a human IgG1 heavy chain constant region. In some embodiments, each polypeptide chain of the antibody heavy chain constant region comprises an amino acid sequence at least 90% identical to the amino acid sequence of wild-type human IgG1 Fc region.

In some embodiments, at least one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

In some embodiments, at least one polypeptide chain of the antibody heavy chain constant region comprises one or more mutations, relative to the amino acid sequence of wild-type human IgG1 Fc region, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

In some embodiments, one polypeptide chain of the antibody heavy chain constant region comprises one or more mutations, relative to the amino acid sequence of wild-type human IgG1 Fc region, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to the amino acid sequence of wild-type human IgG1 Fc region, at one or more positions selected from Q347, Y349, L351, S354, E356, E357, S364, T366, L368, K370, N390, K392, T394, D399, D401, F405, Y407, K409, T411, and K439, numbered according to the EU numbering system.

In some embodiments, one polypeptide chain of the antibody heavy chain constant region comprises K360E and K409W substitutions relative to the amino acid sequence of wild-type human IgG1 Fc region; and the other polypeptide chain of the antibody heavy chain constant region comprises Q347R, D399V and F405T substitutions relative to the amino acid sequence of wild-type human IgG1 Fc region, numbered according to the EU numbering system. In some embodiments, one polypeptide chain of the antibody heavy chain constant region comprises a Y349C substitution relative to the amino acid sequence of wild-type human IgG1 Fc region; and the other polypeptide chain of the antibody heavy chain constant region comprises an S354C substitution relative to the amino acid sequence of wild-type human IgG1 Fc region, numbered according to the EU numbering system.

In another aspect, the present disclosure provides an isolated nucleic acid molecule encoding the antigen-binding sites or proteins described herein.

In another aspect, the present disclosure provides an antibody-drug conjugate comprising the protein provided herein and a drug moiety. In some embodiments, the drug moiety is selected from the group consisting of auristatin, N-acetyl-γ calicheamicin, maytansinoid, pyrrolobenzodiazepine, and SN-38.

In another aspect, the present disclosure provides an immunocytokine comprising the antigen-binding site provided herein and a cytokine. In some embodiments, the cytokine is selected from the group consisting of IL-2, IL-4, IL-10, IL-12, IL-15, TNF, and IFNα.

In another aspect, the present disclosure provides a bispecific T-cell engager comprising the antigen-binding site provided herein and an antigen-binding site that binds CD3 .

In another aspect, the present disclosure provides a chimeric antigen receptor (CAR) comprising: (a) the antigen-binding site provided herein; (b) a transmembrane domain; and (c) an intracellular signaling domain. In some embodiments, transmembrane domain is selected from the transmembrane regions of the alpha, beta or zeta chain of the T-cell receptor, CD28, CD3 epsilon, CD45, CD4, CD5, CD8, CD9, CD16, CD22, 5T4, CD37, CD64, CD80, CD86, CD134, CD137, CD152, and CD154. In some embodiments, the intracellular signaling domain comprises a primary signaling domain comprising a functional cytoplasmic signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In some embodiments, the intracellular signaling domain further comprises a costimulatory signaling domain comprising a functional cytoplasmic signaling domain of a costimulatory receptor. In some embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

In another aspect, the present disclosure provides an isolated nucleic acid molecule encoding the CAR provided herein.

In another aspect, the present disclosure provides an expression vector comprising the isolated nucleic acid provided herein.

In another aspect, the present disclosure provides an immune effector cell comprising the nucleic acid provided herein or the expression vector provided herein. In another aspect, the present disclosure provides an immune effector cell expressing the CAR provided herein. In some embodiments, the immune effector cell is a T cell. In some embodiments, the T cell is a CD8⁺ T cell, a CD4⁺ T cell, a γδ T cell, or an NKT cell. In some embodiments, the immune effector cell is an NK cell.

In another aspect, the present disclosure provides one or more isolated nucleic acid molecules encoding: a VH comprising a CDR1 sequence comprising SEQ ID NO:3, a CDR2 sequence comprising SEQ ID NO:4, and a CDR3 sequence comprising SEQ ID NO:5; and/or a VL comprising a CDR1 sequence comprising SEQ ID NO:6, a CDR2 sequence comprising SEQ ID NO:7, and a CDR3 sequence comprising SEQ ID NO:8.

In another aspect, provided are one or more nucleic acid molecules encoding a VH comprising the amino acid sequence of SEQ ID NO:9; and a VL comprising the amino acid sequence of SEQ ID NO:10.

In another aspect, provided are one or more isolated nucleic acid molecules encoding: a VH comprising the amino acid sequence of SEQ ID NO:11; and a VL comprising the amino acid sequence of SEQ ID NO:12.

In another aspect, the present disclosure provides an isolated polypeptide encoded by the nucleic acid sequence provided herein. In some embodiments, the isolated polypeptide comprises a VH. In some embodiments, the isolated polypeptide comprises a VL. In some embodiments, the isolated polypeptide comprises an scFv.

In another aspect, the present disclosure provides an expression vector comprising the isolated nucleic acid provided herein.

In another aspect, the present disclosure provides a host cell comprising the isolated nucleic acid provided herein or the expression vector provided herein.

In another aspect, the present disclosure provides a cell comprising one or more nucleic acids encoding the antigen-binding site or the antigen-binding domain or the protein provided herein.

In another aspect, the present disclosure provides a pharmaceutical composition comprising the protein provided herein, the antibody-drug conjugate provided herein, the immunocytokine provided herein, the bispecific T-cell engager provided herein, the CAR provided herein, or the immune effector cell provided herein; and a pharmaceutically acceptable carrier.

In another aspect, the present disclosure provides a pharmaceutical composition comprising the protein provided herein, the antibody-drug conjugate provided herein, the immunocytokine provided herein, or the bispecific T cell engager provided herein, and a pharmaceutically acceptable carrier.

In another aspect, the present disclosure provides a method of treating cancer, the method comprising administering to a subject in need thereof an effective amount of the protein provided herein, the antibody-drug conjugate provided herein, the provided herein, the bispecific T-cell engager provided herein, the CAR provided herein, the immune effector cell provided herein, or the pharmaceutical composition provided herein. In some embodiments, the cancer is selected from the group consisting of colorectal cancer, ovarian cancer, non-small cell lung cancer, renal cancer, breast cancer (e.g., hormone receptor positive (HR+) breast cancer), endometrial cancer, squamous cell carcinoma, head and neck squamous cell carcinoma, uterine cancer, pancreatic cancer, mesothelioma, and gastric cancer. In some embodiments, the breast cancer is hormone receptor positive (HR+) breast cancer. In some embodiments, the cancer is a metastatic cancer. In some embodiments, the subject is refractory to chemotherapy. In some embodiments, the method increases overall survival and/or progression free survival in the subject. In some embodiments, the cancer expresses 5T4. In some embodiments, the 5T4 is expressed at high levels relative to normal cells. In some embodiments, the 5T4 is expressed at low levels relative to normal cells.

In another aspect, the present disclosure provides a method of enhancing tumor cell death, the method comprising exposing the tumor cell to an effective amount of the protein provided herein or the pharmaceutical composition provided herein. In another aspect, provided the use of the protein provided herein, the antibody-drug conjugate provided herein, the immunocytokine provided herein, or the bispecific T cell engager provided herein, or a pharmaceutical composition provided herein, for enhancing tumor cell death by exposing the tumor cell to an effective amount of the protein, the antibody-drug conjugate, the immunocytokine, or the bispecific T cell engager, or a pharmaceutical composition comprising such protein, antibody-drug conjugate, immunocytokine, or bispecific T cell engager. In another aspect, provided is the protein provided herein, the antibody-drug conjugate provided herein, the immunocytokine provided herein, or the bispecific T cell engager provided herein, or a pharmaceutical composition provided herein for use in enhancing tumor cell death by exposing the tumor cell to an effective amount of the protein or a pharmaceutical composition comprising such protein, antibody-drug conjugate, immunocytokine, or bispecific T cell engager.

In another aspect, the present disclosure provides a method of enhancing cancer-associated fibroblast (CAF) cell death, the method comprising exposing the CAF to an effective amount of the protein provided herein, the antibody-drug conjugate provided herein, the immunocytokine provided herein, or the bispecific T cell engager provided herein, or the pharmaceutical composition provided herein. In another aspect, provided is the use of the protein provided herein, the antibody-drug conjugate provided herein, the immunocytokine provided herein, or the bispecific T cell engager provided herein, or a pharmaceutical composition provided herein for enhancing cancer-associated fibroblast (CAF) cell death by exposing the CAF to an effective amount of the protein, the antibody-drug conjugate, the immunocytokine, or the bispecific T cell engager, or a pharmaceutical composition comprising such protein, antibody-drug conjugate, immunocytokine, or bispecific T cell engager. In another aspect, provided is the protein provided herein, the antibody-drug conjugate provided herein, the immunocytokine provided herein, or the bispecific T cell engager provided herein, or a pharmaceutical composition provided herein for use in enhancing cancer-associated fibroblast (CAF) cell death by exposing the CAF to an effective amount of the protein, the antibody-drug conjugate, the immunocytokine, or the bispecific T cell engager, or a pharmaceutical composition comprising such protein, antibody-drug conjugate, immunocytokine, or bispecific T cell engager.

In some embodiments, the antigen-binding site provided herein, the protein provided herein, the antibody-drug conjugate provided herein, the immunocytokine provided herein, or the bispecific T cell engager provided herein is a purified antigen-binding site, protein, antibody-drug conjugate, immunocytokine, or bispecific T cell engager. In some embodiments, the antigen-binding site, protein, antibody-drug conjugate, immunocytokine, or bispecific T cell engager is purified by a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

In some embodiments, the protein provided herein is a purified protein. In some embodiments, the protein is purified using a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

These and other aspects and advantages of the invention are illustrated by the following figures, detailed description, and claims.

### BRIEF DESCRIPTION OF THE DRAWINGS

The invention can be more completely understood with reference to the following drawings.

FIGS. 1A-1F are graphs showing surface plasmon resonance (SPR) of multispecific binding proteins to 5T4. FIG. 1A shows binding of AB1310/AB1783 to human 5T4 at pH 7.4. FIG. 1B shows binding of AB0064 to human 5T4 at pH 7.4. FIG. 1C shows binding of AB0064 to cynomolgus 5T4 at pH 7.4. FIG. 1D shows binding of AB0063 to human 5T4 at pH 7.4. FIG. 1E shows binding of AB0063 to cynomolgus 5T4 at pH 7.4. FIG. 1F shows binding of AB 1310/AB 1783 to human 5T4 at pH 7.4.

FIGS. 2A-2C are graphs showing concentration curves showing saturation of binding of AB1310/AB1783 and the parental antibody 10F10 to 5T4-expressing cells. FIG. 2A shows binding to KYSE-30 cells. FIG. 2B shows binding to H292 cells. FIG. 2C shows binding to H2172 cells.

FIGS. 3A-3B are graphs showing binding of AB1310/AB1783 to primary cancer-associated fibroblasts (CAPs). FIG. 3A is a concentration curve showing saturation of binding of AB1310/AB1783 to CAFs. FIG. 3B is a plot showing observed binding EC₅₀ values of AB1310/AB1783 to tumor cells lines and primary CAPs.

FIG. 4 are flow cytometry dot plots of a polyspecificity assay showing AB1310/AB1783 (left panels) or controls (center and right panels) in the absence (top panels) or presence (bottom panels) of poly-specificity reagent (PSR).

FIGS. 5A-5D are graphs showing binding (fold over background (FOB)) of various concentrations of 10F 10, 11F09, and mutants thereof produced via humanization and sequence liability correction to 5T4⁺ H1975 cells.

FIG. 5E and FIG. 5F are graphs showing binding (fold over background (FOB)) of various concentrations of humanized 5T4 binders.

### DETAILED DESCRIPTION

The present disclosure provides antigen-binding sites that bind human 5T4. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating 5T4-associated diseases such as cancer. Various aspects of the present disclosure are set forth in the sections below; however, aspects of the invention described in one particular section are not to be limited to any particular section.

To facilitate an understanding of the present disclosure, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in or is capable of antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering unless otherwise indicated.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by Kabat et al., J. Biol. Chem. 252, 6609-6616 (1977) and Kabat et al., Sequences of protein of immunological interest. (1991) (“Kabat” numbering scheme); Chothia et al., J. Mol. Biol. 196:901-917 (1987), Al-Lazikani et al., 1997, J. Mol. Biol., 273:927-948 (“Chothia” numbering scheme); Lefranc et al., Dev. Comp. Immunol., 2003, 27:55-77 (“IMGT” numbering scheme); Honegger and Plückthun, J. Mol. Biol., 2001, 309:657-70 (“AHo” or “Honegger” numbering scheme), MacCallum et al., J. Mol. Biol. 262:732-745 (1996) (“Contact” numbering scheme) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991), each of which is incorporated by reference in its entirety. In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs. CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

The term “protein” as used herein means a macromolecule that comprises one or more chains of amino acids. Such a chain of amino acids may be referred to as a polypeptide, which is a continuous, unbranched chain of amino acids linked by peptide bonds. Accordingly, a protein may include a single polypeptide or multiple polypeptides.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

With respect to treatment of cancer, a 5T4-targeting antigen-binding site or antigen-binding domain, as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, a 5T4-targeting antigen-binding site or antigen-binding domain, as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

An “increased” or “enhanced” amount (e.g., with respect to cancer cell proliferation or expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein. In various embodiments, tumor burden is determined using linear dimensional methods (e.g. Response Evaluation Criteria in Solid Tumors (RECIST) v1.1 (Eisenhauer, et al., Eur J Cancer. (2009) 45(2):228-47). In various embodiments, tumor burden is determined using volumetric analysis (e.g., positron emission tomography (PET) / computed tomography (CT) scan). See, e.g., Paydary, et al., Mol Imaging Biol. (2019) 21(1):1-10; Li, et al., AJR Am J Roentgenol. (2021) 217(6):1433-1443; and Kerner, et al., EJNMMI Res. (2016) Dec;6(1):33.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence or recurrence of a tumor, e.g., a relapse after remission.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington’s Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present disclosure which, upon administration to a subject, is capable of providing a compound of this invention or an active metabolite or residue thereof. As is known to those of skill in the art, “salts” of the compounds of the present disclosure may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the present disclosure and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH4⁺, and NW₄⁺ (wherein W is a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, 5T4 (also known as Trophoblast glycoprotein, TPBG, Wnt-activated Inhibitory Factor 1, WAIF1, M6P1, 5T4AG) refers to the protein of Uniprot Accession No. Q13641 and related isoforms and orthologs. The NCBI Gene ID for 5T4 is 7162.

As used herein, the terms “specific binding,” “specifically binds to,” “specific for,” “selectively binds,” and “selective for,” with regard to the binding of a protein or antigen-binding site as described herein to a target molecule, a particular antigen (e.g., a polypeptide target), or an epitope on a particular antigen, mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the protein or antigen-binding site to the target molecule is competitively inhibited by the control molecule. A protein or antigen-binding site as described herein that “specifically binds to” or is “specific for” a particular polypeptide or an epitope on a particular polypeptide is one that binds to that particular polypeptide or epitope on a particular polypeptide without substantially binding to any other polypeptide or polypeptide epitope. In some instances, the protein or antigen-binding site as described herein specifically binds to an antigen, e.g., a polypeptide target, with dissociation constant (K_{D}) as described herein, for example, in the form of an antibody, scFv, Fab, or other form of a protein described herein measured at a temperature of about 4° C., 25° C., 37° C., or 42° C. Affinities of a protein or antigen-binding site as described herein can be readily determined using conventional techniques, for example, those described by Scatchard et al., Ann. N. Y. Acad. Sci. USA, 51:660 (1949), ELISA assays, biolayer interferometry (BLI) assays, and surface plasmon resonance (SPR) assays. Binding properties of a protein or antigen-binding site as described herein to antigens, cells, or tissues thereof may generally be determined and assessed using immunodetection methods including, for example, immunofluorescence-based assays, such as immuno-histochemistry (IHC) and/or fluorescence- activated cell sorting (FACS). Generally, but not necessarily, reference to “binding” means “specific binding.”

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the invention are discussed in more detail below.

### I. Antigen-Binding Site

In one aspect, the present disclosure provides an antigen-binding site that binds human 5T4. The VH, VL, CDR, and scFv sequences of exemplary antigen-binding sites are listed in Table 1. The CDR sequences are identified according to the Chothia numbering scheme, unless otherwise specified. Table 1A provides CDR sequences according to Kabat numbering scheme. Table 1B provides CDR sequences according to Chothia numbering scheme. Table 1C provides CDR sequences according to IMGT numbering scheme. Table 1D provides CDR sequences according to Honegger numbering scheme.

TABLE 1

Sequences of Exemplary Antigen-Binding Sites that Bind 5T4

| | | |
|---|---|---|
| Clone | VH | VL |
| 10F10 murine | EVQLQESGGGLVKPGGSRRLSCA ASRFTFSDFGMHWVRQAPEKGLE WIAYISSGSSTIYYADTVKGRFT ISRDNAKNTLFLQMTSLRSEDTA MYYCASSQSYYRGTLDYWGQGTS VTVSS (SEQ ID NO:1) | DIVLTQSPASLAVTLGQRATISCRA SQSVTTSNYNYMHWFHQKPGQPPKL LIKFASNLESGVPARFSGSGSGTDF TLNIHPVEEEDIATYYCQHSWEIPW TFGGGTKLEIK (SEQ ID NO:2) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO:8) |
| AB 1002 (humanized | EVQLVESGGGLVKPGGSLRLSCA ASRFTFSDFGMHWVRQAPGKGLE | DIQLTQSPSSLSASVGDRVTITCRA SQSVTTSNYNYMHWFQQKPGKAPKL |
| 10F10, T62S) | WVSYISSGSSTIYYADSVKGRFT ISRDNAKNSLYLQMNSLRAEDTA VYYCASSQSYYRGTLDYWGQGTT VTVSS (SEQ ID NO:9) | LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| AB 1002 (with cysteine heterodimer ization mutations for disulfide bond formation) | EVQLVESGGGLVKPGGSLRLSCA ASRFTFSDFGMHWVRQAPGKCLE WVSYISSGSSTIYYADSVKGRFT ISRDNAKNSLYLQMNSLRAEDTA VYYCASSQSYYRGTLDYWGQGTT VTVSS (SEQ ID NO:11) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGCGTKVEIK (SEQ ID NO:12) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| AB1002 scFv (VL-VH) (with cysteine heterodimer ization mutations for disulfide bond formation) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASRFTFSDFGMHWVRQAPGKCLEWVSYISSGSSTIYYADSVKGRFT ISRDNAKNSLYLQMNSLRAEDTAVYYCASSQSYYRGTLDYWGQGTTVTVS S (SEQ ID NO:13) | |
| AB1002 scFv (VH-VL) (with cysteine heterodimer ization mutations | EVQLVESGGGLVKPGGSLRLSCAASRFTFSDFGMHWVRQAPGKCLEWVSY ISSGSSTIYYADSVKGRFTISRDNAKNSLYLQMNSLRAEDTAVYYCASSQ SYYRGTLDYWGQGTTVTVSSGGGGSGGGGSGGGGSGGGGSDIQLTQSPSS LSASVGDRVTITCRASQSVTTSNYNYMHWFQQKPGKAPKLLIKFASNLES GVPSRFSGSGSGTDFTLTISSLQPEDFATYYCQHSWEIPWTFGCGTKVEI K (SEQ ID NO:14) | |
| for disulfide bond formation) | | |
| AB 1002 (humanized 10F10) M33L | EVQLVESGGGLVKPGGSLRLSCA ASRFTFSDFGMHWVRQAPGKGLE WVSYISSGSSTIYYADSVKGRFT ISRDNAKNSLYLQMNSLRAEDTA VYYCASSQSYYRGTLDYWGQGTT VTVSS (SEQ ID NO:9) | DIQLTQSPSSLSASVGDRVTITCRA SQSVTTSNYNYLHWFQQKPGKAPKL LIKFASNLESGVPSRFSGSGSGTDF TLTISSLQPEDFATYYCQHSWEIPW TFGGGTKVEIK (SEQ ID NO:129) |
| | CDR1- RFTFSDF (SEQ ID NO:3) | CDR1- RASQSVTTSNYNYLH (SEQ ID NO:130) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| AB 1002 (humanized 10F10) M33V | EVQLVESGGGLVKPGGSLRLSCA ASRFTFSDFGMHWVRQAPGKGLE WVSYISSGSSTIYYADSVKGRFT ISRDNAKNSLYLQMNSLRAEDTA VYYCASSQSYYRGTLDYWGQGTT VTVSS (SEQ ID NO:9) | DIQLTQSPSSLSASVGDRVTITCRA SQSVTTSNYNYVHWFQQKPGKAPKL LIKFASNLESGVPSRFSGSGSGTDF TLTISSLQPEDFATYYCQHSWEIPW TFGGGTKVEIK (SEQ ID NO:131) |
| | CDR1- RFTFSDF (SEQ ID NO:3) | CDR1- RASQSVTTSNYNYVH (SEQ ID NO:132) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 05H04 | QVQLVQSGAEVKKPGASVKVSCK ASGYKFTDYYMDWVRQAPGQGLE WIGYIFPNDASTTYNEKFKGKAT LTADKSTNTAYMELSSLRSEDTA VYYCARSRDADYWGQGTTVTVSS (SEQ ID NO:15) | ENVLTQSPATLSLSPGERATLSCSA KSSVSYIHWYQQKPGQAPRLLIYDT SYLGSGIPARFSGSGSGTDYTLTIS SLEPEDFAVYYCQQWSSYPYTFGGG TKVEIK (SEQ ID NO:16) |
| | CDR1- GYKFTDY (SEQ ID NO:41) | CDR1- SAKSSVSYIH (SEQ ID NO:44) |
| | CDR2- FPNDAS (SEQ ID NO:42) | CDR2- DTSYLGS (SEQ ID NO:45) |
| | CDR3- SRDADY (SEQ ID NO:43) | CDR3- QQWSSYPYT (SEQ ID NO:46) |
| 05H04 Q1E | EVQLVQSGAEVKKPGASVKVSCK ASGYKFTDYYMDWVRQAPGQGLE WIGYIFPNDASTTYNEKFKGKAT LTADKSTNTAYMELSSLRSEDTA VYYCARSRDADYWGQGTTVTVSS (SEQ ID NO:17) | ENVLTQSPATLSLSPGERATLSCSA KSSVSYIHWYQQKPGQAPRLLIYDT SYLGSGIPARFSGSGSGTDYTLTIS SLEPEDFAVYYCQQWSSYPYTFGGG TKVEIK (SEQ ID NO:16) |
| | CDR1- GYKFTDY (SEQ ID NO:41) | CDR1- SAKSSVSYIH (SEQ ID NO:44) |
| | CDR2- FPNDAS (SEQ ID NO:42) | CDR2- DTSYLGS (SEQ ID NO:45) |
| | CDR3- SRDADY (SEQ ID NO:43) | CDR3- QQWSSYPYT (SEQ ID NO:46) |
| 05H04 scFv (VH-VL) | QVQLVQSGAEVKKPGASVKVSCKASGYKFTDYYMDWVRQAPGQCLEWIGY IFPNDASTTYNEKFKGKATLTADKSTNTAYMELSSLRSEDTAVYYCARSR DADYWGQGTTVTVSSGGGGSGGGGSGGGGSGGGGSENVLTQSPATLSLSP GERATLSCSAKSSVSYIHWYQQKPGQAPRLLIYDTSYLGSGIPARFSGSG SGTDYTLTISSLEPEDFAVYYCQQWSSYPYTFGCGTKVEIK (SEQ ID NO:18) | |
| 05H04 scFv (VL-VH) | ENVLTQSPATLSLSPGERATLSCSAKSSVSYIHWYQQKPGQAPRLLIYDT SYLGSGIPARFSGSGSGTDYTLTISSLEPEDFAVYYCQQWSSYPYTFGCG TKVEIKGGGGSGGGGSGGGGSGGGGSQVQLVQSGAEVKKPGASVKVSCKA SGYKFTDYYMDWVRQAPGQCLEWIGYIFPNDASTTYNEKFKGKATLTADK STNTAYMELSSLRSEDTAVYYCARSRDADYWGQGTTVTVSS (SEQ ID NO:120) | |
| 05H04 Q1E (VH-VL) | EVQLVQSGAEVKKPGASVKVSCKASGYKFTDYYMDWVRQAPGQCLEWIGY IFPNDASTTYNEKFKGKATLTADKSTNTAYMELSSLRSEDTAVYYCARSR DADYWGQGTTVTVSSGGGGSGGGGSGGGGSGGGGSENVLTQSPATLSLSP GERATLSCSAKSSVSYIHWYQQKPGQAPRLLIYDTSYLGSGIPARFSGSG SGTDYTLTISSLEPEDFAVYYCQQWSSYPYTFGCGTKVEIK (SEQ ID NO:19) | |
| 11F09 | EVQLVESGGGLVKPGGSLRLSCE ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:20) | DIQLTQSPSSLSASVGDRVTITCRA SQGVSTSTYTYLHWFQQKPGKAPKL LIKFASNLESGVPSRFSGSGSGTDF TLTISSLQPEDFATYYCQHSWEIPW TFGGGTKVEIK (SEQ ID NO:21) |
| | CDR1- GFTFSDY (SEQ ID NO:47) | CDR1- RASQGVSTSTYTYLH (SEQ ID NO:49) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 10F10 21*05 AB1002 parental humanized (T62) | EVQLVESGGGLVKPGGSLRLSCA ASRFTFSDFGMHWVRQAPGKGLE WVSYISSGSSTIYYADTVKGRFT ISRDNAKNSLYLQMNSLRAEDTA VYYCASSQSYYRGTLDYWGQGTT VTVSS (SEQ ID NO:22) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 21*05 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASRFTFSDFGMHWVRQAPGKCLEWVSYISSGSSTIYYADTVKGRFT ISRDNAKNSLYLQMNSLRAEDTAVYYCASSQSYYRGTLDYWGQGTTVTVS S (SEQ ID NO:23) | |
| 10F10 23*03 humanized variant 2 | EVQLLESGGGLVQPGGSLRLSCA ASRFTFSDFGMHWVRQAPGKGLE WIAYISSGSSTIYYADTVKGRFT ISRDNAKNTLYLQMNSLRAEDTA VYYCASSQSYYRGTLDYWGQGTT VTVSS (SEQ ID NO:24) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 23*03 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLLESGGGLVQPGGSLR LSCAASRFTFSDFGMHWVRQAPGKCLEWIAYISSGSSTIYYADTVKGRFT ISRDNAKNTLYLQMNSLRAEDTAVYYCASSQSYYRGTLDYWGQGTTVTVS S (SEQ ID NO:25) | |
| 10F10 23*03 BM1 | EVQLLESGGGLVQPGGSLRLSCA ASRFTFSDFGMHWVRQAPGKGLE WVSYISSGSSTIYYADTVKGRFT | DIQLTQSPSSLSASVGDRVTITCRA SQSVTTSNYNYMHWFQQKPGKAPKL LIKFASNLESGVPSRFSGSGSGTDF |
| | ISRDNSKNTLYLQMNSLRAEDTA VYYCASSQSYYRGTLDYWGQGTT VTVSS (SEQ ID NO:108) | TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 23*03 BM1 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLLESGGGLVQPGGSLR LSCAASRFTFSDFGMHWVRQAPGKCLEWVSYISSGSSTIYYADTVKGRFT ISRDNSKNTLYLQMNSLRAEDTAVYYCASSQSYYRGTLDYWGQGTTVTVS S (SEQ ID NO:133) | |
| 10F10 48*01 humanized variant 3 | EVQLVESGGGLVQPGGSLRLSCA ASRFTFSDFGMHWVRQAPGKGLE WVSYISSGSSTIYYADTVKGRFT ISRDNAKNSLYLQMNSLRAEDTA VYYCASSQSYYRGTLDYWGQGTT VTVSS (SEQ ID NO:138) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 48*01 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVQPGGSLR LSCAASRFTFSDFGMHWVRQAPGKCLEWVSYISSGSSTIYYADTVKGRFT ISRDNAKNSLYLQMNSLRAEDTAVYYCASSQSYYRGTLDYWGQGTTVTVS S (SEQ ID NO:27) | |
| 10F10 48*01 BM2 | EVQLVESGGGLVQPGGSLRLSCA ASRFTFSDFGMHWVRQAPGKGLE WIAYISSGSSTIYYADTVKGRFT ISRDNAKNSLYLQMNSLRAEDTA VYYCASSQSYYRGTLDYWGQGTT VTVSS (SEQ ID NO:26) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 48*01 BM2 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVQPGGSLR LSCAASRFTFSDFGMHWVRQAPGKCLEWIAYISSGSSTIYYADTVKGRFT ISRDNAKNSLYLQMNSLRAEDTAVYYCASSQSYYRGTLDYWGQGTTVTVS S (SEQ ID NO:121) | |
| 10F10 11*01 humanized variant 4 | QVQLVESGGGLVKPGGSLRLSCA ASRFTFSDFGMHWVRQAPGKGLE WIAYISSGSSTIYYADTVKGRFT ISRDNAKNSLYLQMNSLRAEDTA VYYCASSQSYYRGTLDYWGQGTT VTVSS (SEQ ID NO:28) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 11*01 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSQVQLVESGGGLVKPGGSLR LSCAASRFTFSDFGMHWVRQAPGKCLEWIAYISSGSSTIYYADTVKGRFT ISRDNAKNSLYLQMNSLRAEDTAVYYCASSQSYYRGTLDYWGQGTTVTVS S (SEQ ID NO:29) | |
| 10F10 21*05 humanized variant 5 | EVQLVESGGGLVKPGGSLRLSCA ASRFTFSDFGMHWVRQAPGKGLE WIAYISSGSSTIYYADTVKGRFT ISRDNAKNSLYLQMNSLRAEDTA VYYCASSQSYYRGTLDYWGQGTT VTVSS (SEQ ID NO:30) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO: 8) |
| 10F10 21*05 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASRFTFSDFGMHWVRQAPGKCLEWIAYISSGSSTIYYADTVKGRFT ISRDNAKNSLYLQMNSLRAEDTAVYYCASSQSYYRGTLDYWGQGTTVTVS S (SEQ ID NO:31) | |
| 10F10 11*01 BM1 | EVQLVESGGGLVKPGGSLRLSCA ASRFTFSDFGMHWIRQAPGKGLE WVSYISSGSSTIYYADTVKGRFT ISRDNAKNSLYLQMNSLRAEDTA VYYCASSQSYYRGTLDYWGQGTT VTVSSG (SEQ ID NO:106) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO: 10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | CDR3- SQSYYRGTLDY (SEQ ID NO:5) | CDR3- QHSWEIPWT (SEQ ID NO:8) |
| 10F10 11*01 scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASRFTFSDFGMHWIRQAPGKCLEWVSYISSGSSTIYYADTVKGRFT ISRDNAKNSLYLQMNSLRAEDTAVYYCASSQSYYRGTLDYWGQGTTVTVS S (SEQ ID NO:107) | |
| 10F10 humanized consensus sequence | X₁VQLX₂ESGGGLVX₃PGGSLRLS CAASRFTFSDFGMHWVRQAPGKG LEWX₄X₅YISSGSSTIYYADX₆VK GRFTISRDNAKNX₇LYLQMNSLR AEDTAVYYCASSQSYYRGTLDYW GQGTTVTVSS (SEQ ID NO:94) | DIQLTQSPSSLSASVGDRVTITCRA SQSVTTSNYNYMHWFQQKPGKAPKL LIKFASNLESGVPSRFSGSGSGTDF TLTISSLQPEDFATYYCQHSWEIPW TFGGGTKVEIK (SEQ ID NO:10) |
| | CDR1- RFTFSDF (SEQ ID NO:3) | CDR1- RASQSVTTSNYNYMH (SEQ ID NO:6) |
| | CDR2- SSGSST (SEQ ID NO:4) | CDR2- FASNLES (SEQ ID NO:7) |
| | | CDR3- QHSWEIPWT (SEQ ID NO: 8) |
| | CDR3- SQSYYRGTLDY (SEQ ID NO:5) | |
| | where X₁ is E or Q; X₂ is V or L; X₃ is K or Q; X₄ is V or I; X₅ is S or A; X₆ is S or T; and X₇ is S or T | |
| 10F10 humanized consensus scFv (VH-VL) (with cysteine mutations for disulfide bond formation) | X₁VQLX₂ESGGGLVX₃PGGSLRLSCAASRFTFSDFGMHWVRQAPGKCLEWX ₄X₅YISSGSSTIYYADX₆VKGRFTISRDNAKNX₇LYLQMNSLRAEDTAVYY CASSQSYYRGTLDYWGQGTTVTVSSGGGGSGGGGSGGGGSGGGGSDIQLT QSPSSLSASVGDRVTITCRASQSVTTSNYNYMHWFQQKPGKAPKLLIKFA SNLESGVPSRFSGSGSGTDFTLTISSLQPEDFATYYCQHSWEIPWTFGCG TKVEIK (SEQ ID NO:95) | |
| | where X₁ is E or Q; X₂ is V or L; X₃ is K or Q; X₄ is V or I; X₅ is S or A; X₆ is S or T; and X₇ is S or T | |
| 10F10 humanized consensus scFv (VL-VH) (with cysteine mutations for disulfide bond formation) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSX₁VQLX₂ESGGGLVX₃PGGS LRLSCAASRFTFSDFGMHWVRQAPGKCLEWX₄X₅YISSGSSTI YYADX_{g}VK GRFTISRDNAKNX₇LYLQMNSLRAEDTAVYYCASSQSYYRGTLDYWGQGT TVTVSS (SEQ ID NO:96) | |
| | where X₁ is E or Q; X₂ is V or L; X₃ is K or Q; X₄ is V or I; X₅ is S or A; X₆ is S or T; and X₇ is S or T | |
| 10F10 humanized consensus scFv (VH-VL) | X₁VQLX₂ESGGGLVX₃PGGSLRLSCAASRFTFSDFGMHWVRQAPGKGLEWX ₄X₅YISSGSSTIYYADX₆VKGRFTISRDNAKNX₇LYLQMNSLRAEDTAVYY CASSQSYYRGTLDYWGQGTTVTVSSGGGGSGGGGSGGGGSGGGGSDIQLT QSPSSLSASVGDRVTITCRASQSVTTSNYNYMHWFQQKPGKAPKLLIKFA SNLESGVPSRFSGSGSGTDFTLTISSLQPEDFATYYCQHSWEIPWTFGGG TKVEIK (SEQ ID NO:134) | |
| | Where X₁ is E or Q; X₂ is V or L; X₃ is K or Q; X₄ is V or I; X₅ is S or A; X₆ is S or T; and X₇ is S or T | |
| 10F10 humanized consensus scFv (VL-VH) | DIQLTQSPSSLSASVGDRVTITCRASQSVTTSNYNYMHWFQQKPGKAPKL LIKFASNLESGVPSRFSGSGSGTDFTLTISSLQPEDFATYYCQHSWEIPW TFGGGTKVEIKGGGGSGGGGSGGGGSGGGGSX₁VQLX₂ESGGGLVX₃PGGS LRLSCAASRFTFSDFGMHWVRQAPGKGLEWX₄X₅YISSGSSTI YYADX_{g}VK GRFTISRDNAKNX₇LYLQMNSLRAEDTAVYYCASSQSYYRGTLDYWGQGT TVTVSS (SEQ ID NO:135) | |
| | Where X₁ is E or Q; X₂ is V or L; X₃ is K or Q; X₄ is V or I; X₅ is S or A; X₆ is S or T; and X₇ is S or T | |
| 11F09 48*01 | EVQLVESGGGLVQPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:32) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 48*01 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVQPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:34) | |
| 11F09 48*01 BM2 | EVQLVESGGGLVQPGGSLRLSCA ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:122) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 48*01 BM2 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVQPGGSLR LSCAASGFTFSDYGMHWVRQAPGKCLEWLAYISSGSSTFYYADTVKGRFT ISRDNAKNSLYLQMNSLRAEDTAVYYCGSSQSYYRGTMDYWGQGTTVTVS S (SEQ ID NO:123) | |
| 11F09 21*05 | EVQLVESGGGLVKPGGSLRLSCE ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:20) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 21*05 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCEASGFTFSDYGMHWVRQAPGKCLEWLAYISSGSSTFYYADTVKGRFT ISRDNAKNSLYLQMNSLRAEDTAVYYCGSSQSYYRGTMDYWGQGTTVTVS S (SEQ ID NO:36) | |
| 11F09 21*05 BM1 | EVQLVESGGGLVKPGGSLRLSCE ASGFTFSDYGMHWVRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:139) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 21*05 scFv (VL-VH) BM1 | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCEASGFTFSDYGMHWVRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:124) | |
| 11F09 11*01 | QVQLVESGGGLVKPGGSLRLSCA ASGFTFSDYGMHWVRQAPGKGLE WLAYISSGSSTFYYADTVKGRFT ISRDNAKNSLYLQMNSLRAEDTA | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW |
| | VYYCGSSQSYYRGTMDYWGQGTT VTVSS (SEQ ID NO:37) | TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 11*01 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSQVQLVESGGGLVKPGGSLR LSCAASGFTFSDYGMHWVRQAPGKCLEWLAYISSGSSTFYYADTVKGRFT ISRDNAKNSLYLQMNSLRAEDTAVYYCGSSQSYYRGTMDYWGQGTTVTVS S (SEQ ID NO:38) | |
| 11F09 11*01 BM1 | EVQLVESGGGLVKPGGSLRLSCA ASGFTFSDYGMHWIRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:125) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 11*01 BM1 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWIRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:126) | |
| 11F09 23*03 | EVQLLESGGGLVQPGGSLRLSCA ASGFTFSDYGMHWVRQAPGKGLE WVSYISSGSSTFYYADTVKGRFT ISRDNSKNTLYLQMNSLRAEDTA VYYCGSSQSYYRGTMDYWGQGTT VTVSS (SEQ ID NO:39) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 23*03 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLLESGGGLVQPGGSLR LSCAASGFTFSDYGMHWVRQAPGKCLEWVSYISSGSSTFYYADTVKGRFT ISRDNSKNTLYLQMNSLRAEDTAVYYCGSSQSYYRGTMDYWGQGTTVTVS S (SEQ ID NO:40) | |
| 11F09 23*03-BM2 | EVQLLESGGGLVQPGGSLRLSCA ASGFTFSDYGMHWVRQAPGKGLE WLAYISSGSSTFYYADTVKGRFT ISRDNAKNTLYLQMNSLRAEDTA VYYCGSSQSYYRGTMDYWGQGTT VTVSS (SEQ ID NO:127) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1- GFTFSDY (SEQ ID NO:47) | CDR1- RASQGVSTSTYTYMH (SEQ ID NO:50) |
| | CDR2- SSGSST (SEQ ID NO:4) | CDR2- FASNLES (SEQ ID NO:7) |
| | CDR3- SQSYYRGTMDY (SEQ ID NO:48) | CDR3- QHSWEIPWT (SEQ ID NO: 8) |
| 11F09 23*03-BM2 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLLESGGGLVQPGGSLR LSCAASGFTFSDYGMHWVRQAPGKCLEWLAYISSGSSTFYYADTVKGRFT ISRDNAKNTLYLQMNSLRAEDTAVYYCGSSQSYYRGTMDYWGQGTTVTVS S (SEQ ID NO:128) | |
| 08E06 | EVQLQESGAEMVRPGASVKLSCK ASGYTFTSYWMHWVKQRPGQGLE WIGKIDSSDSKTQYNQKFKDKAT LTVDKSSITVYMELNSLTSEDSA VYYCARGGYLWFAYWGQGTLVTV SA (SEQ ID NO:51) | DIVMTQSPASLAVSLGQRATISCRA SQSVSTSTYSYMHWYQQKPGQSPKL LITYASNLESGVPARFSGSGSGTDF TLNIHPVEEEDIATYYCQHSWEIPW TFGGGTKLEIK (SEQ ID NO:52) |
| | CDR1- GYTFTSY (SEQ ID NO:53) | CDR1- RASQSVSTSTYSYMH (SEQ ID NO:56) |
| | CDR2- DSSDSK (SEQ ID NO:54) | CDR2- YASNLES (SEQ ID NO:57) |
| | CDR3- GGYLWFAY (SEQ ID NO:55) | CDR3- QHSWEIPWT (SEQ ID NO: 8) |
| 08E06-humanized variant 1 | QVQLVQSGAEVKKPGASVKVSCK ASGYTFTSYWMHWVRQAPGQGLE WMGKIDSSDSKTQYNQKFKDRVT MTRDTSTSTVYMELSSLRSEDTA VYYCARGGYLWFAYWGQGTLVTV SS (SEQ ID NO:58) | DIVMTQSPDSLAVSLGERATINCRA SQSVSTSTYSYMHWYQQKPGQPPKL LIYYASNLESGVPDRFSGSGSGTDF TLTISSLQAEDVAVYYCQHSWEIPW TFGGGTKVEIK (SEQ ID NO:59) |
| | CDR1- GYTFTSY (SEQ ID NO:53) | CDR1- RASQSVSTSTYSYMH (SEQ ID NO:56) |
| | CDR2- DSSDSK (SEQ ID NO:54) | CDR2- YASNLES (SEQ ID NO:57) |
| | CDR3- GGYLWFAY (SEQ ID NO:55) | CDR3- QHSWEIPWT (SEQ ID NO: 8) |
| scFv08E06 (VH-VL) humanized variant 1 (with cysteine mutations for disulfide bond formation) | QVQLVQSGAEVKKPGASVKVSCKASGYTFTSYWMHWVRQAPGQCLEWMGK IDSSDSKTQYNQKFKDRVTMTRDTSTSTVYMELSSLRSEDTAVYYCARGG YLWFAYWGQGTLVTVSSGGGGSGGGGSGGGGSGGGGSDIVMTQSPDSLAV SLGERATINCRASQSVSTSTYSYMHWYQQKPGQPPKLLIYYASNLESGVP DRFSGSGSGTDFTLTISSLQAEDVAVYYCQHSWEIPWTFGCGTKVEIK (SEQ ID NO:60) | |
| scFv08E06 (VL-VH) humanized variant 1 (with cysteine mutations for disulfide bond formation) | DIVMTQSPDSLAVSLGERATINCRASQSVSTSTYSYMHWYQQKPGQPPKL LIYYASNLESGVPDRFSGSGSGTDFTLTISSLQAEDVAVYYCQHSWEIPW TFGCGTKVEIKGGGGSGGGGSGGGGSGGGGSQVQLVQSGAEVKKPGASVK VSCKASGYTFTSYWMHWVRQAPGQCLEWMGKIDSSDSKTQYNQKFKDRVT MTRDTSTSTVYMELSSLRSEDTAVYYCARGGYLWFAYWGQGTLVTVSS (SEQ ID NO:61) | |
| scFv 08E06 (VH-VL) humanized variant 1 | QVQLVQSGAEVKKPGASVKVSCKASGYTFTSYWMHWVRQAPGQGLEWMGK IDSSDSKTQYNQKFKDRVTMTRDTSTSTVYMELSSLRSEDTAVYYCARGG YLWFAYWGQGTLVTVSSGGGGSGGGGSGGGGSGGGGSDIVMTQSPDSLAV SLGERATINCRASQSVSTSTYSYMHWYQQKPGQPPKLLIYYASNLESGVP DRFSGSGSGTDFTLTISSLQAEDVAVYYCQHSWEIPWTFGGGTKVEIK (SEQ ID NO:136) | |
| scFv08E06 (VL-VH) | DIVMTQSPDSLAVSLGERATINCRASQSVSTSTYSYMHWYQQKPGQPPKL LIYYASNLESGVPDRFSGSGSGTDFTLTISSLQAEDVAVYYCQHSWEIPW TFGGGTKVEIKGGGGSGGGGSGGGGSGGGGSQVQLVQSGAEVKKPGASVK VSCKASGYTFTSYWMHWVRQAPGQGLEWMGKIDSSDSKTQYNQKFKDRVT | |
| humanized variant 1 | MTRDTSTSTVYMELSSLRSEDTAVYYCARGGYLWFAYWGQGTLVTVSS (SEQ ID NO:137) | |
| 08E06-humanized variant 2 | QVQLVQSGAEVKKPGASVKVSCK ASGYTFTSYWMHWVRQAPGQGLE WMGKIDSSDSKTQYNQKFKDRVT MTVDTSSSTVYMELSSLRSEDTA VYYCARGGYLWFAYWGQGTLVTV S (SEQ ID NO:62) | DIVMTQSPASLAVSLGERATINCRA SQSVSTSTYSYMHWYQQKPGQPPKL LITYASNLESGVPDRFSGSGSGTDF TLTISSLQAEDVATYYCQHSWEIPW TFGGGTKVEIK (SEQ ID NO:63) |
| | CDR1- GYTFTSY (SEQ ID NO:53) | CDR1- RASQSVSTSTYSYMH (SEQ ID NO:56) |
| | CDR2- DSSDSK (SEQ ID NO:54) | CDR2- YASNLES (SEQ ID NO:57) |
| | CDR3- GGYLWFAY (SEQ ID NO:55) | CDR3- QHSWEIPWT (SEQ ID NO: 8) |
| AB1011 (11F09-VH_BM1-VK_BM1) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:90) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1 - GFTFSDY (SEQ ID NO:47) | CDR1 - RASQGVSTSTYTYMH (SEQ ID NO:50) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1011 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:97) | |
| AB1012 (11F09-VH_BM1-VK_BM1_ M33L) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:90) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:21) |
| | CDR1 - GFTFSDY (SEQ ID NO:47) | CDR1 - RASQGVSTSTYTYLH (SEQ ID NO:49) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1012 scFv (VL-VH) | DIQLTQSPSSLSASVGDRVTITCRASQGVSTSTYTYLHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:98) | |
| AB1013 (11F09-VH_BM1-VK_BM1_ M33V) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTMDYWGQGTT VTVSS (SEQ ID NO:90) | DIQLTQSPSSLSASVGDRVTITCRA SQGVSTSTYTYVHWFQQKPGKAPKL LIKFASNLESGVPSRFSGSGSGTDF TLTISSLQPEDFATYYCQHSWEIPW TFGGGTKVEIK (SEQ ID NO:91) |
| | CDR1 - GFTFSDY (SEQ ID NO:47) | CDR1 - RASQGVSTSTYTYVH (SEQ ID NO:92) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1013 scFv (VL-VH) | DIQLTQSPSSLSASVGDRVTITCRASQGVSTSTYTYVHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTMDYWGQGTTVTVS S (SEQ ID NO:99) | |
| AB1014 (11F09-VH_BM1_ M100cI-VK_BM1) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTIDYWGQGTT VTVSS (SEQ ID NO:93) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1 - GFTFSDY (SEQ ID NO:47) | CDR1 - RASQGVSTSTYTYMH (SEQ ID NO:50) |
| | CDR2 - SSGSST (SEQ ID NO:4) | CDR2 - FASNLES (SEQ ID NO:7) |
| | CDR3 - SQSYYRGTIDY (SEQ ID NO:35) | CDR3 - QHSWEIPWT (SEQ ID NO: 8) |
| AB1014 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTIDYWGQGTTVTVS S (SEQ ID NO:100) | |
| AB1015 (11F09-VH_BM1_ M100cI-VK_BM1_ M33L) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTIDYWGQGTT VTVSS (SEQ ID NO:93) | DIQLTQSPSSLSASVGDRVTITCRA SQGVSTSTYTYLHWFQQKPGKAPKL LIKFASNLESGVPSRFSGSGSGTDF TLTISSLQPEDFATYYCQHSWEIPW TFGGGTKVEIK (SEQ ID NO:21) |
| | CDR1 - GFTFSDY (SEQ ID NO:47) | CDR1 - RASQGVSTSTYTYLH (SEQ ID NO:49) |
| | CDR2 - SSGSST (SEQ ID NO:4) | CDR2 - FASNLES (SEQ ID NO:7) |
| | CDR3 - SQSYYRGTIDY (SEQ ID NO:35) | CDR3 - QHSWEIPWT (SEQ ID NO: 8) |
| AB1015 scFv (VL-VH) | DIQLTQSPSSLSASVGDRVTITCRASQGVSTSTYTYLHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTIDYWGQGTTVTVS S (SEQ ID NO:101) | |
| AB1016 (11F09-VH_BM1_ M100cI-VK_BM1_ M33V) | EVQLVESGGGLVKPGGSLRLSCA ASGFTFSDYGMHWVRQAPGKGLE WVSYISSGSSTFYYADTVKGRFT ISRDNAKNSLYLQMNSLRAEDTA VYYCGSSQSYYRGTIDYWGQGTT VTVSS (SEQ ID NO:93) | DIQLTQSPSSLSASVGDRVTITCRA SQGVSTSTYTYVHWFQQKPGKAPKL LIKFASNLESGVPSRFSGSGSGTDF TLTISSLQPEDFATYYCQHSWEIPW TFGGGTKVEIK (SEQ ID NO:91) |
| | CDR1 - GFTFSDY (SEQ ID NO:47) | CDR1 - RASQGVSTSTYTYVH (SEQ ID NO:92) |
| | CDR2 - SSGSST (SEQ ID NO:4) | CDR2 - FASNLES (SEQ ID NO:7) |
| | CDR3 - SQSYYRGTIDY (SEQ ID NO:35) | CDR3 - QHSWEIPWT (SEQ ID NO: 8) |
| AB1016 scFv (VL-VH) | DIQLTQSPSSLSASVGDRVTITCRASQGVSTSTYTYVHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCAASGFTFSDYGMHWVRQAPGKCLEWVSYISSGSSTFYYADTVKGRFT ISRDNAKNSLYLQMNSLRAEDTAVYYCGSSQSYYRGTIDYWGQGTTVTVS S (SEQ ID NO:102) | |
| AB1017 (11F09-VH_BM2-VK_BM1) | EVQLVESGGGLVKPGGSLRLSCE ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:20) | DIQLTQSPSSLSASVGDRVTITCRA SQGVSTSTYTYMHWFQQKPGKAPKL LIKFASNLESGVPSRFSGSGSGTDF TLTISSLQPEDFATYYCQHSWEIPW TFGGGTKVEIK (SEQ ID NO:33) |
| | CDR1 - GFTFSDY (SEQ ID NO:47) | CDR1 - RASQGVSTSTYTYMH (SEQ ID NO:50) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1017 scFv (VL-VH) | DIQLTQSPSSLSASVGDRVTITCRASQGVSTSTYTYMHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCEASGFTFSDYGMHWVRQAPGKCLEWLAYISSGSSTFYYADTVKGRFT ISRDNAKNSLYLQMNSLRAEDTAVYYCGSSQSYYRGTMDYWGQGTTVTVS S (SEQ ID NO:103) | |
| AB1018 (11F09-VH_BM2-VK_BM1_ M33L) | EVQLVESGGGLVKPGGSLRLSCE ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:20) | DIQLTQSPSSLSASVGDRVTITCRA SQGVSTSTYTYLHWFQQKPGKAPKL LIKFASNLESGVPSRFSGSGSGTDF TLTISSLQPEDFATYYCQHSWEIPW TFGGGTKVEIK (SEQ ID NO:21) |
| | CDR1 - GFTFSDY (SEQ ID NO:47) | CDR1 - RASQGVSTSTYTYLH (SEQ ID NO:49) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1018 scFv (VL-VH) | DIQLTQSPSSLSASVGDRVTITCRASQGVSTSTYTYLHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCEASGFTFSDYGMHWVRQAPGKCLEWLAYISSGSSTFYYADTVKGRFT | |
| | ISRDNAKNSLYLQMNSLRAEDTAVYYCGSSQSYYRGTMDYWGQGTTVTVS S (SEQ ID NO:104) | |
| AB1019 (11F09-VH_BM2-VK_BM1_ M33V) | EVQLVESGGGLVKPGGSLRLSCE ASGFTFSDYGMHWVRQAPGKGLE WLAYISSGSSTFYYADTVKGRFT ISRDNAKNSLYLQMNSLRAEDTA VYYCGSSQSYYRGTMDYWGQGTT VTVSS (SEQ ID NO:20) | DIQLTQSPSSLSASVGDRVTITCRA SQGVSTSTYTYVHWFQQKPGKAPKL LIKFASNLESGVPSRFSGSGSGTDF TLTISSLQPEDFATYYCQHSWEIPW TFGGGTKVEIK (SEQ ID NO:91) |
| | CDR1 - GFTFSDY (SEQ ID NO:47) | CDR1 - RASQGVSTSTYTYVH (SEQ ID NO:92) |
| | CDR2 - SSGSST (SEQ ID NO:4) | CDR2 - FASNLES (SEQ ID NO:7) |
| | CDR3 - SQSYYRGTMDY (SEQ ID NO:48) | CDR3 - QHSWEIPWT (SEQ ID NO: 8) |
| AB1019 scFv (VL-VH) | DIQLTQSPSSLSASVGDRVTITCRASQGVSTSTYTYVHWFQQKPGKAPKL LIKFASNLESGVPSRFSGSGSGTDFTLTISSLQPEDFATYYCQHSWEIPW TFGCGTKVEIKGGGGSGGGGSGGGGSGGGGSEVQLVESGGGLVKPGGSLR LSCEASGFTFSDYGMHWVRQAPGKCLEWLAYISSGSSTFYYADTVKGRFT ISRDNAKNSLYLQMNSLRAEDTAVYYCGSSQSYYRGTMDYWGQGTTVTVS S (SEQ ID NO:105) | |

TABLE 1A

CDRs for illustrative anti-5T4 binding antigen binding domains (Kabat)

| Ab Name | VH - CDR1 | VH - CDR2 | VH - CDR3 | VL - CDR1 | VL - CDR2 | VL - CDR3 |
|---|---|---|---|---|---|---|
| 140 | DFGMH SEQ ID NO:170 | YISSGSSTIYYADTVKG SEQ ID NO:171 | SQSYYRGTLDY SEQ ID NO:5 | RASQSVTTSNYNYMH SEQ ID NO:6 | FASNLES SEQ ID NO: 7 | QHSWEIPWT SEQ ID NO:8 |
| 141 | DFGMH SEQ ID NO:170 | YISSGSSTIYYADSVKG SEQ ID NO:172 | SQSYYRGTLDY SEQ ID NO:5 | RASQSVTTSNYNYMH SEQ ID NO:6 | FASNLES SEQ ID NO: 7 | QHSWEIPWT SEQ ID NO:8 |
| 142 | DFGMH SEQ ID NO:170 | YISSGSSTIYYADSVKG SEQ ID NO:172 | SQSYYRGTLDY SEQ ID NO:5 | RASQSVTTSNYNYLH SEQ ID NO:130 | FASNLES SEQ ID NO: 7 | QHSWEIPWT SEQ ID NO:8 |
| 143 | DFGMH SEQ ID NO:170 | YISSGSSTIYYADSVKG SEQ ID NO:172 | SQSYYRGTLDY SEQ ID NO:5 | RASQSVTTSNYNYVH SEQ ID NO:132 | FASNLES SEQ ID NO: 7 | QHSWEIPWT SEQ ID NO:8 |
| 144 | DFGMH SEQ ID NO:170 | YISSGSSTIYYADX₆VKG X₆=S or T SEQ ID NO:173 | SQSYYRGTLDY SEQ ID NO:5 | RASQSVTTSNYNYX₈H X₈=L, M or V SEQ ID NO:174 | FASNLES SEQ ID NO: 7 | QHSWEIPWT SEQ ID NO:8 |
| 145 | DYYMD SEQ ID NO:175 | YIFPNDASTTYNEKFKG SEQ ID NO:176 | SRDADY SEQ ID NO:177 | SAKSSVSYIH SEQ ID NO:44 | DTSYLGS SEQ ID NO: 45 | QQWSSYPYT SEQ ID NO:46 |
| 146 | DYGMH SEQ ID NO:178 | YISSGSSTFYYADTVKG SEQ ID NO:179 | SQSYYRGTMDY SEQ ID NO:48 | RASQGVSTSTYTYLH SEQ ID NO:49 | FASNLES SEQ ID NO: 7 | QHSWEIPWT SEQ ID NO:8 |
| 147 | DYGMH SEQ ID NO:178 | YISSGSSTFYYADTVKG SEQ ID NO:179 | SQSYYRGTMDY SEQ ID NO:48 | RASQGVSTSTYTYMH SEQ ID NO:50 | FASNLES SEQ ID NO: 7 | QHSWEIPWT SEQ ID NO:8 |
| 148 | DYGMH SEQ ID NO:178 | YISSGSSTFYYADTVKG SEQ ID NO:179 | SQSYYRGTMDY SEQ ID NO:48 | RASQGVSTSTYTYVH SEQ ID NO:92 | FASNLES SEQ ID NO: 7 | QHSWEIPWT SEQ ID NO:8 |
| 149 | DYGMH SEQ ID NO:178 | YISSGSSTFYYADTVKG SEQ ID NO:179 | SQSYYRGTIDY SEQ ID NO:35 | RASQGVSTSTYTYMH SEQ ID NO:50 | FASNLES SEQ ID NO: 7 | QHSWEIPWT SEQ ID NO:8 |
| 150 | DYGMH SEQ ID NO:178 | YISSGSSTFYYADTVKG SEQ ID NO:179 | SQSYYRGTIDY SEQ ID NO:35 | RASQGVSTSTYTYLH SEQ ID NO:49 | FASNLES SEQ ID NO: 7 | QHSWEIPWT SEQ ID NO:8 |
| 151 | DYGMH SEQ ID NO:178 | YISSGSSTFYYADTVKG SEQ ID NO:179 | SQSYYRGTIDY SEQ ID NO:35 | RASQGVSTSTYTYVH SEQ ID NO:92 | FASNLES SEQ ID NO: 7 | QHSWEIPWT SEQ ID NO:8 |
| 152 | SYWMH SEQ ID NO:180 | KIDSSDSKTQYNQKFKD SEQ ID NO:181 | GGYLWFAY SEQ ID NO:55 | RASQSVSTSTYSYMH SEQ ID NO:56 | YASNLES SEQ ID NO:57 | QHSWEIPWT SEQ ID NO:8 |

TABLE 1B

CDRs for illustrative anti-5T4 binding antigen binding domains (Chothia)

| Ab Name | VH - CDR1 | VH - CDR2 | VH - CDR3 | VL - CDR1 | VL -CDR2 | VL - CDR3 |
|---|---|---|---|---|---|---|
| 153 | RFTFSDF SEQ ID NO:3 | SGSS SEQ ID NO:182 | QSYYRGTLD SEQ ID NO:183 | SQSVTTSNYNY SEQ ID NO:184 | FAS SEQ ID NO:185 | SWEIPW SEQ ID NO:186 |
| 154 | GYKFTDY SEQ ID NO:41 | PNDA SEQ ID NO:187 | RDAD SEQ ID NO:188 | KSSVSY SEQ ID NO:189 | DTS SEQ ID NO:190 | WSSYPY SEQ ID NO:191 |
| 155 | GFTFSDY SEQ ID NO:47 | SGSS SEQ ID NO:182 | QSYYRGTMD SEQ ID NO:192 | SQGVSTSTYTY SEQ ID NO:193 | FAS SEQ ID NO:185 | SWEIPW SEQ ID NO:186 |
| 156 | GFTFSDY SEQ ID NO:47 | SGSS SEQ ID NO:182 | QSYYRGTID SEQ ID NO:194 | SQGVSTSTYTY SEQ ID NO:193 | FAS SEQ ID NO:185 | SWEIPW SEQ ID NO:186 |
| 157 | GYTFTSY SEQ ID NO:53 | SSDS SEQ ID NO:195 | GYLWFA SEQ ID NO:196 | SQSVSTSTYSY SEQ ID NO:197 | YAS SEQ ID NO:198 | SWEIPW SEQ ID NO:186 |

TABLE 1C

CDRs for illustrative anti-5T4 binding antigen binding domains (IMGT)

| Ab Name | VH - CDR1 | VH - CDR2 | VH - CDR3 | VL - CDR1 | VL -CDR2 | VL - CDR3 |
|---|---|---|---|---|---|---|
| 158 | RFTFSDFG SEQ ID NO:199 | ISSGSSTI SEQ ID NO:200 | ASSQSYYRGTLDY SEQ ID NO:201 | QSVTTSNYNY SEQ ID NO:202 | FAS SEQ ID NO:185 | QHSWEIPWT SEQ ID NO:8 |
| 159 | GYKFTDYY SEQ ID NO:203 | IFPNDAST SEQ ID NO:204 | ARSRDADY SEQ ID NO:205 | SSVSY SEQ ID NO:206 | DTS SEQ ID NO:190 | QQWSSYPYT SEQ ID NO:46 |
| 160 | GFTFSDYG SEQ ID NO:207 | ISSGSSTF SEQ ID NO:208 | GSSQSYYRGTMDY SEQ ID NO:209 | QGVSTSTYTY SEQ ID NO:210 | FAS SEQ ID NO:185 | QHSWEIPWT SEQ ID NO:8 |
| 161 | GFTFSDYG SEQ ID NO:207 | ISSGSSTF SEQ ID NO:208 | GSSQSYYRGTIDY SEQ ID NO:211 | QGVSTSTYTY SEQ ID NO:210 | FAS SEQ ID NO:185 | QHSWEIPWT SEQ ID NO:8 |
| 162 | GYTFTSYW SEQ ID NO:212 | IDSSDSKT SEQ ID NO:213 | ARGGYLWFAY SEQ ID NO:214 | QSVSTSTYSY SEQ ID NO:215 | YAS SEQ ID NO:198 | QHSWEIPWT SEQ ID NO:8 |

TABLE 1D

CDRs for illustrative anti-5T4 binding antigen binding domains (Honegger)

| Ab Name | VH - CDR1 | VH - CDR2 | VH - CDR3 | VL - CDR1 | VL - CDR2 | VL -CDR3 |
|---|---|---|---|---|---|---|
| 163 | ASRFTFSDFG SEQ ID NO:216 | ISSGSSTIYYADTVKGR SEQ ID NO:217 | SQSYYRGTLD SEQ ID NO:218 | ASQSVTTSNYNY SEQ ID NO:219 | FASNLESGVPAR SEQ ID NO:220 | SWEIPW SEQ ID NO:186 |
| 164 | ASRFTFSDFG SEQ ID NO:216 | ISSGSSTIYYADSVKGR SEQ ID NO:221 | SQSYYRGTLD SEQ ID NO:218 | ASQSVTTSNYNY SEQ ID NO:219 | FASNLESGVPSR SEQ ID NO:222 | SWEIPW SEQ ID NO:186 |
| 165 | ASRFTFSDFG SEQ ID NO:216 | ISSGSSTIYYADX₆VKGR X₆ = S or T SEQ ID NO:223 | SQSYYRGTLD SEQ ID NO:218 | ASQSVTTSNYNY SEQ ID NO:219 | FASNLESGVPSR SEQ ID NO:222 | SWEIPW SEQ ID NO:186 |
| 166 | ASRFTFSDFG SEQ ID NO:216 | ISSGSSTIYYADX₆VKGR X₆ = S or T SEQ ID NO:223 | SQSYYRGTLD SEQ ID NO:218 | ASQSVTTSNYNY SEQ ID NO:219 | FASNLESGVPX₉R X₉ = A or S SEQ ID NO:245 | SWEIPW SEQ ID NO:186 |
| 167 | ASGYKFTDYY SEQ ID NO:224 | IFPNDASTTYNEKFKGK SEQ ID NO:225 | SRDAD SEQ ID NO:226 | AKSSVSY SEQ ID NO:227 | DTSYLGSGIPAR SEQ ID NO:228 | WSSYPY SEQ ID NO:191 |
| 168 | ASGFTFSDYG SEQ ID NO:229 | ISSGSSTFYYADTVKGR SEQ ID NO:230 | SQSYYRGTMD SEQ ID NO:231 | ASQGVSTSTYTY SEQ ID NO:232 | FASNLESGVPSR SEQ ID NO:222 | SWEIPW SEQ ID NO:186 |
| 169 | ASGFTFSDYG SEQ ID NO:229 | ISSGSSTFYYADTVKGR SEQ ID NO:230 | SQSYYRGTID SEQ ID NO:233 | ASQGVSTSTYTY SEQ ID NO:232 | FASNLESGVPSR SEQ ID NO:222 | SWEIPW SEQ ID NO:186 |
| 170 | ASGYTFTSYW SEQ ID NO:234 | IDSSDSKTQYNQKFKDK SEQ ID NO:235 | GGYLWFA SEQ ID NO:236 | ASQSVSTSTYSY SEQ ID NO:237 | YASNLESGVPAR SEQ ID NO:238 | SWEIPW SEQ ID NO:186 |
| 171 | ASGYTFTSYW SEQ ID NO:234 | IDSSDSKTQYNQKFKDR SEQ ID NO:239 | GGYLWFA SEQ ID NO:236 | ASQSVSTSTYSY SEQ ID NO:237 | YASNLESGVPDR SEQ ID NO:240 | SWEIPW SEQ ID NO:186 |

In certain embodiments, the antigen-binding site or antigen-binding domain of the present disclosure comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the antigen-binding site or antigen-binding domain that binds to 5T4 comprises an antibody heavy chain variable domain (VH) that comprises an amino acid sequence at least 95% identical to the VH of an antibody disclosed in Table 1, and an antibody light chain variable domain (VL) that comprises an amino acid sequence at least 95% identical to the VL of the same antibody disclosed in Table 1. In certain embodiments, the antigen-binding site or antigen-binding domain that binds to 5T4 comprises an antibody heavy chain variable domain (VH) that comprises an amino acid sequence at least 96% identical to the VH of an antibody disclosed in Table 1, and an antibody light chain variable domain (VL) that comprises an amino acid sequence at least 96% identical to the VL of the same antibody disclosed in Table 1. In certain embodiments, the antigen-binding site or antigen-binding domain that binds to 5T4 comprises an antibody heavy chain variable domain (VH) that comprises an amino acid sequence at least 97% identical to the VH of an antibody disclosed in Table 1, and an antibody light chain variable domain (VL) that comprises an amino acid sequence at least 97% identical to the VL of the same antibody disclosed in Table 1. In certain embodiments, the antigen-binding site or antigen-binding domain that binds to 5T4 comprises an antibody heavy chain variable domain (VH) that comprises an amino acid sequence at least 98% identical to the VH of an antibody disclosed in Table 1, and an antibody light chain variable domain (VL) that comprises an amino acid sequence at least 98% identical to the VL of the same antibody disclosed in Table 1. In certain embodiments, the antigen-binding site or antigen-binding domain that binds to 5T4 comprises an antibody heavy chain variable domain (VH) that comprises an amino acid sequence at least 99% identical to the VH of an antibody disclosed in Table 1, and an antibody light chain variable domain (VL) that comprises an amino acid sequence at least 99% identical to the VL of the same antibody disclosed in Table 1. In certain embodiments, the antigen-binding site or antigen-binding domain that binds to 5T4 comprises an antibody heavy chain variable domain (VH) that comprises the amino acid sequence of an antibody disclosed in Table 1, and an antibody light chain variable domain (VL) that comprises the amino acid sequence of an antibody disclosed in Table 1. Sequence identity can be determined according to the BLAST algorithm (blast.ncbi.nlm.nih.gov/Blast.cgi), using default settings.

In certain embodiments, the antigen-binding site comprises the heavy chain CDR1, CDR2, and CDR3, and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), or any other CDR determination method known in the art, of the VH and VL sequences of an antibody disclosed in Table 1. In certain embodiments, the antigen-binding site comprises the heavy chain CDR1, CDR2, and CDR3, and the light chain CDR1, CDR2, and CDR3 of an antibody disclosed in Tables 1, 1A, 1B, 1C or 1D.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Kabat), respectively:
SEQ ID NOs: 170, 171, 5, 6, 7 and 8;
SEQ ID NOs: 170, 172, 5, 6, 7 and 8;
SEQ ID NOs: 170, 172, 5, 130, 7 and 8;
SEQ ID NOs: 170, 172, 5, 132, 7 and 8;
SEQ ID NOs: 170, 173, 5, 174, 7 and 8;
SEQ ID NOs: 175, 176, 177, 44, 45 and 46;
SEQ ID NOs: 178, 179, 48, 49, 7 and 8;
SEQ ID NOs: 178, 179, 48, 50, 7 and 8;
SEQ ID NOs: 178, 179, 48, 92, 7 and 8;
SEQ ID NOs: 178, 179, 35, 50, 7 and 8;
SEQ ID NOs: 178, 179, 35, 49, 7 and 8;
SEQ ID NOs: 178, 179, 35, 92, 7 and 8; or
SEQ ID NOs: 180, 181, 55, 56, 57 and 8.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Kabat), respectively:
SEQ ID NOs: 170, 171, 5, 6, 7 and 8;
SEQ ID NOs: 170, 172, 5, 6, 7 and 8;
SEQ ID NOs: 170, 172, 5, 130, 7 and 8;
SEQ ID NOs: 170, 172, 5, 132, 7 and 8; or
SEQ ID NOs: 170, 173, 5, 174, 7 and 8.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Chothia), respectively:
SEQ ID NOs: 3, 182, 183, 184, 185 and 186;
SEQ ID NOs: 41, 187, 188, 189, 190 and 191;
SEQ ID NOs: 47, 182, 192, 193, 185 and 186;
SEQ ID NOs: 47, 182, 194, 193, 185 and 186; or
SEQ ID NOs: 53, 195, 196, 197, 198 and 186.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Chothia), respectively: SEQ ID NOs: 3, 182, 183, 184, 185 and 186.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to IMGT), respectively:
SEQ ID NOs: 199, 200, 201, 202, 185 and 8;
SEQ ID NOs: 203, 204, 205, 206, 190 and 46;
SEQ ID NOs: 207, 208, 209, 210, 185 and 8;
SEQ ID NOs: 207, 208, 211, 210, 185 and 8; or
SEQ ID NOs: 212, 213, 214, 215, 198 and 8;

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to IMGT), respectively: SEQ ID NOs: 199, 200, 201, 202, 185 and 8.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Honegger), respectively:
SEQ ID NOs: 216, 217, 218, 219, 220 and 186;
SEQ ID NOs: 216, 221, 218, 219, 222 and 186;
SEQ ID NOs: 216, 223, 218, 219, 222 and 186;
SEQ ID NOs: 216, 223, 218, 219, 245 and 186;
SEQ ID NOs: 224, 225, 226, 227, 228 and 191;
SEQ ID NOs: 229, 230, 231, 232, 222 and 186;
SEQ ID NOs: 234, 235, 236, 237, 238 and 186; or
SEQ ID NOs: 234, 239, 236, 237, 240 and 186.

In various embodiments, the antigen binding site or antigen-binding domain that binds 5T4 (e.g., human 5T4) comprises a VH-CDR1, a VH-CDR2, a VH-CDR3, a VL-CDR1, a VL-CDR2, and a VL-CDR3 comprising the following amino acid sequences (according to Honegger), respectively:
SEQ ID NOs: 216, 217, 218, 219, 220 and 186;
SEQ ID NOs: 216, 221, 218, 219, 222 and 186;
SEQ ID NOs: 216, 223, 218, 219, 222 and 186; or
SEQ ID NOs: 216, 223, 218, 219, 245 and 186.

In certain embodiments, the VH comprises CDR1, CDR2, and CDR3 comprising the amino acid sequences of SEQ ID NOs: 3, 4, and 5, respectively. In certain embodiments, the VL comprises CDR1, CDR2, and CDR3 comprising the amino acid sequences of SEQ ID NOs: 6, 7, and 8, respectively.

In certain embodiments, the antigen-binding site of the present disclosure is derived from 10F10. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 130, 7, and 8, respectively. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 132, 7, and 8, respectively. In certain embodiments, the antigen-binding site comprises (a) a VH that comprises a CDR1, CDR2, and CDR3 comprising the amino acid sequences of SEQ ID NOs: 170, 172, and 5, respectively; and (b) a VL that comprises a CDR1, CDR2, and CDR3 comprising the amino acid sequences of SEQ ID NOs: 6, 7, and 8, respectively. In certain embodiments, the antigen-binding site comprises (a) a VH that comprises a CDR1, CDR2, and CDR3 comprising the amino acid sequences of SEQ ID NOs: 3, 182, and 183, respectively; and (b) a VL that comprises a CDR1, CDR2, and CDR3 comprising the amino acid sequences of SEQ ID NOs: 184, 185, and 186, respectively. In certain embodiments, the antigen-binding site comprises (a) a VH that comprises a CDR1, CDR2, and CDR3 comprising the amino acid sequences of SEQ ID NOs: 199, 200, and 201, respectively; and (b) a VL that comprises a CDR1, CDR2, and CDR3 comprising the amino acid sequences of SEQ ID NOs: 202, 185, and 8, respectively. In certain embodiments, the antigen-binding site comprises (a) a VH that comprises a CDR1, CDR2, and CDR3 comprising the amino acid sequences of SEQ ID NOs: 216, 217, and 218, respectively; and (b) a VL that comprises a CDR1, CDR2, and CDR3 comprising the amino acid sequences of SEQ ID NOs: 219, 220, and 186, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:2.

In certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:94, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 95 or 96.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 134 or 135.

In certain embodiments, the antigen-binding site of the present disclosure is derived from AB1002. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In some embodiments, the antigen-binding site of the present disclosure comprises a VH that comprises an amino acid sequence at least 95% identical to the amino acid sequence of SEQ ID NO:9, and a VL that comprises an amino acid sequence at least 95% identical to the amino acid sequence of SEQ ID NO: 10. In some embodiments, the antigen-binding site of the present disclosure comprises a VH that comprises an amino acid sequence at least 96% identical to the amino acid sequence of SEQ ID NO:9, and a VL that comprises an amino acid sequence at least 96% identical to the amino acid sequence of SEQ ID NO: 10. In some embodiments, the antigen-binding site of the present disclosure comprises a VH that comprises an amino acid sequence at least 97% identical to the amino acid sequence of SEQ ID NO:9, and a VL that comprises an amino acid sequence at least 97% identical to the amino acid sequence of SEQ ID NO:10. In some embodiments, the antigen-binding site of the present disclosure comprises a VH that comprises an amino acid sequence at least 98% identical to the amino acid sequence of SEQ ID NO:9, and a VL that comprises an amino acid sequence at least 98% identical to the amino acid sequence of SEQ ID NO:10. In some embodiments, the antigen-binding site of the present disclosure comprises a VH that comprises an amino acid sequence at least 99% identical to the amino acid sequence of SEQ ID NO:9, and a VL that comprises an amino acid sequence at least 99% identical to the amino acid sequence of SEQ ID NO:10. In certain embodiments, the VH comprises a substitution of cysteine (C) at position 44 (G44C) relative to SEQ ID NO:9, according to the Kabat numbering scheme. In certain embodiments, the VL comprises a substitution of cysteine (C) at position 100 (G100C) relative to SEQ ID NO:10, according to the Kabat numbering scheme. In certain embodiments, the VL comprises a substitution of leucine (L) at position 33 (M33L), according to the Kabat numbering scheme (SEQ ID NO:130). In certain embodiments, the VL comprises a substitution of valine (V) at position 33 (M33V), according to the Kabat numbering scheme (SEQ ID NO:132). An exemplary VL comprising the M33L substitution is SEQ ID NO:129. An exemplary VL comprising the M33V substitution is SEQ ID NO:131. Accordingly, in certain embodiments, the antigen-binding site of the present disclosure comprises a VH that comprises an amino acid sequence that is at least 90% (e.g., at least 91%, at least 92%, at least 93%, at least 94%, at least 95%, at least 96%, at least 97%, at least 98%, at least 99%, or 100%) identical to a VH derived from 10F10, and a VL that comprises an amino acid sequence that is at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:129 or SEQ ID NO:131.

In certain embodiments, the antigen-binding site or antigen-binding domain of the present disclosure comprises a VH that comprises an amino acid sequence that is at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:11, and a VL that comprises an amino acid sequence that is at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:12. In some embodiments, the antigen-binding site or antigen-binding domain comprises a VH that comprises an amino acid sequence that is at least 95% identical to the amino acid sequence of SEQ ID NO:11, and a VL that comprises an amino acid sequence that is at least 95% identical to the amino acid sequence of SEQ ID NO:12. In some embodiments, the antigen-binding site or antigen-binding domain comprises a VH that comprises an amino acid sequence that is at least 96% identical to the amino acid sequence of SEQ ID NO:11, and a VL that comprises an amino acid sequence that is at least 96% identical to the amino acid sequence of SEQ ID NO:12. In some embodiments, the antigen-binding site or antigen-binding domain comprises a VH that comprises an amino acid sequence that is at least 97% identical to the amino acid sequence of SEQ ID NO:11, and a VL that comprises an amino acid sequence that is at least 97% identical to the amino acid sequence of SEQ ID NO:12. In some embodiments, the antigen-binding site or antigen-binding domain comprises a VH that comprises an amino acid sequence that is at least 98% identical to the amino acid sequence of SEQ ID NO:11, and a VL that comprises an amino acid sequence that is at least 98% identical to the amino acid sequence of SEQ ID NO:12. In some embodiments, the antigen-binding site or antigen-binding domain comprises a VH that comprises an amino acid sequence that is at least 99% identical to the amino acid sequence of SEQ ID NO:11, and a VL that comprises an amino acid sequence that is at least 99% identical to the amino acid sequence of SEQ ID NO:12. In some embodiments, the antigen-binding site or antigen-binding domain comprises a VH comprising the amino acid sequence of SEQ ID NO:11, and a VL comprising the amino acid sequence of SEQ ID NO:12.

In certain embodiments, the antigen-binding site or antigen-binding domain is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 13 or 14. In some embodiments, the scFv comprises an amino acid sequence at least 95% identical to the amino acid sequence of SEQ ID NO: 13 or 14.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:13. In some embodiments, the scFv comprises an amino acid sequence at least 95% identical to the amino acid sequence of SEQ ID NO:13. In some embodiments, the scFv comprises an amino acid sequence at least 96% identical to the amino acid sequence of SEQ ID NO:13. In some embodiments, the scFv comprises an amino acid sequence at least 97% identical to the amino acid sequence of SEQ ID NO:13. In some embodiments, the scFv comprises an amino acid sequence at least 98% identical to the amino acid sequence of SEQ ID NO:13. In some embodiments, the scFv comprises an amino acid sequence at least 99% identical to the amino acid sequence of SEQ ID NO: 13. In some embodiments, the scFv comprises an amino acid sequence of SEQ ID NO:13.

In certain embodiments, the VH comprises a CDR1, CDR2, and CDR3 comprising the amino acid sequences of SEQ ID NOs: 41, 42, and 43, respectively. In certain embodiments, the VL comprises a CDR1, CDR2, and CDR3 comprising the amino acid sequences of SEQ ID NOs: 44, 45, and 46, respectively.

In certain embodiments, the antigen-binding site of the present disclosure is derived from 05H04. In certain embodiments, the antigen-binding site comprises (a) a VH that comprises a CDR1, CDR2, and CDR3 comprising the amino acid sequences of SEQ ID NOs: 41, 42, and 43, respectively; and (b) a VL that comprises a CDR1, CDR2, and CDR3 comprising the amino acid sequences of SEQ ID NOs: 44, 45, and 46, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 15, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 16. In certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 17, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 16.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 18 or 19. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 120.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 49, 7, and 8, respectively.

In certain embodiments, the antigen-binding site of the present disclosure is derived from 11F09. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 49, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21.

In certain embodiments, the antigen-binding site of the present disclosure is derived from 10F10 21*05 AB1002 parental humanized (T62). In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:22, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:23.

In certain embodiments, the antigen-binding site of the present disclosure is derived from 10F10 23*03 humanized variant 2. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:24, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:25.

In certain embodiments, the antigen-binding site of the present disclosure is derived from 10F10 23*03 BM1. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 108, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 133,

In certain embodiments, the antigen-binding site of the present disclosure is derived from 10F10 48*01 BM2. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:26, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 121.

In certain embodiments, the antigen-binding site of the present disclosure is derived from 10F10 48*01 humanized variant 3. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 138, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:27.

In certain embodiments, the antigen-binding site of the present disclosure is derived from 10F10 11*01 humanized variant 1. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:28, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29.

In certain embodiments, the antigen-binding site of the present disclosure is derived from 10F10 11*01 BM1. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 106, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 107.

In certain embodiments, the antigen-binding site of the present disclosure is derived from 10F10 21*05 humanized variant 5. In certain embodiments, the antigen-binding site comprises (a) a VH that comprises a CDR1, CDR2, and CDR3 comprising the amino acid sequences of SEQ ID NOs: 3, 4, and 5, respectively; and (b) a VL that comprises a CDR1, CDR2, and CDR3 comprising the amino acid sequences of SEQ ID NOs: 6, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:30, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the VH comprises a substitution of serine (S) at position 62, according to the Kabat numbering scheme.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:31.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 50, 7, and 8, respectively.

In certain embodiments, the antigen-binding site of the present disclosure is derived from 11F09 48*01. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:32, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:34.

In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively. In certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 122, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 123.

In certain embodiments, the antigen-binding site of the present disclosure is derived from 11F09 21*05. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:36.

In certain embodiments, the antigen-binding site of the present disclosure is derived from 11F09 21*05 BM1. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively. In certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 139, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 124.

In certain embodiments, the antigen-binding site of the present disclosure is derived from 11F09 11 *01. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:38.

In certain embodiments, the antigen-binding site of the present disclosure is derived from 11F09 11*01 BM1. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively. For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 125, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 126.

In certain embodiments, the antigen-binding site of the present disclosure is derived from 11F09 23*03. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:39, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:40.

In certain embodiments, the antigen-binding site of the present disclosure is derived from 11F09 23*03 BM2. In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 127, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 128.

In certain embodiments, the VH comprises a CDR1, CDR2, and CDR3 comprising the amino acid sequences of SEQ ID NOs: 53, 54, and 55, respectively. In certain embodiments, the VL comprises a CDR1, CDR2, and CDR3 comprising the amino acid sequences of SEQ ID NOs: 56, 57, and 8, respectively.

In certain embodiments, the antigen-binding site of the present disclosure is derived from 08E06. In certain embodiments, the antigen-binding site comprises (a) a VH that comprises a CDR1, CDR2, and CDR3 comprising the amino acid sequences of SEQ ID NOs: 53, 54, and 55, respectively; and (b) a VL that comprises a CDR1, CDR2, and CDR3 comprising the amino acid sequences of SEQ ID NOs: 56, 57, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:51, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:52.

In certain embodiments, the antigen-binding site of the present disclosure is derived from 08E06-humanized variant 1. In certain embodiments, the antigen-binding site comprises (a) a VH that comprises a CDR1, CDR2, and CDR3 comprising the amino acid sequences of SEQ ID NOs: 53, 54, and 55, respectively; and (b) a VL that comprises a CDR1, CDR2, and CDR3 comprising the amino acid sequences of SEQ ID NOs: 56, 57, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:59.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NOs: 60 or 61.

In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 136 or 137.

In certain embodiments, the antigen-binding site of the present disclosure is derived from 08E06-humanized variant 2. In certain embodiments, the antigen-binding site comprises (a) a VH that comprises a CDR1, CDR2, and CDR3 comprising the amino acid sequences of SEQ ID NOs: 53, 54, and 55, respectively; and (b) a VL that comprises a CDR1, CDR2, and CDR3 comprising the amino acid sequences of SEQ ID NOs: 56, 57, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:62, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:63.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 50, 7, and 8, respectively.

In certain embodiments, the antigen-binding site of the present disclosure is derived from AB1011 (11F09-VH_BM1-VK_BM1). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NOVO, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:97.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 49, 7, and 8, respectively.

In certain embodiments, the antigen-binding site of the present disclosure is derived from AB1012 (11F09-VH BM1-VK BM1 M33L). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 49, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:90, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 92, 7, and 8, respectively.

In certain embodiments, the antigen-binding site of the present disclosure is derived from AB1013 (11F09-VH BM1-VK BM1 M33V). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 92, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NOVO, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:99.

In certain embodiments, the antigen-binding site of the present disclosure is derived from AB1014 (11F09-VH BM1 M100cI-VK BM1). In certain embodiments, the antigen-binding site comprises (a) a VH that comprises a CDR1, CDR2, and CDR3 comprising the amino acid sequences of SEQ ID NOs: 47, 4, and 35, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the VH comprises a CDR1, CDR2, and CDR3 comprising the amino acid sequences of SEQ ID NOs: 47, 4, and 35, respectively. In certain embodiments, the VL comprises a CDR1, CDR2, and CDR3 comprising the amino acid sequences of SEQ ID NOs: 50, 7, and 8, respectively.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 100.

In certain embodiments, the antigen-binding site of the present disclosure is derived from AB1015 (11F09-VH BM1 M100cI-VK BM1 M33L). In certain embodiments, the antigen-binding site comprises (a) a VH that comprises a CDR1, CDR2, and CDR3 comprising the amino acid sequences of SEQ ID NOs: 47, 4, and 35, respectively; and (b) a VL that comprises a CDR1, CDR2, and CDR3 comprising the amino acid sequences of SEQ ID NOs: 49, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21. In certain embodiments, the VH comprises a CDR1, CDR2, and CDR3 comprising the amino acid sequences of SEQ ID NOs: 47, 4, and 35, respectively. In certain embodiments, the VL comprises a CDR1, CDR2, and CDR3 comprising the amino acid sequences of SEQ ID NOs: 49, 7, and 8, respectively.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 101.

In certain embodiments, the antigen-binding site of the present disclosure is derived from AB1016 (11F09-VH BM1 M100cI-VK BM1 M33V). In certain embodiments, the antigen-binding site comprises (a) a VH that comprises a CDR1, CDR2, and CDR3 comprising the amino acid sequences of SEQ ID NOs: 47, 4, and 35, respectively; and (b) a VL that comprises a CDR1, CDR2, and CDR3 comprising the amino acid sequences of SEQ ID NOs: 92, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91. In certain embodiments, the VH comprises a CDR1, CDR2, and CDR3 comprising the amino acid sequences of SEQ ID NOs: 47, 4, and 35, respectively. In certain embodiments, the VL comprises a CDR1, CDR2, and CDR3 comprising the amino acid sequences of SEQ ID NOs: 92, 7, and 8, respectively.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 102.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 50, 7, and 8, respectively.

In certain embodiments, the antigen-binding site of the present disclosure is derived from AB1017 (11F09-VH_BM2-VK_BM1). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 50, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 103.

In certain embodiments, the VH comprises CDR1, CDR2, and CDR3 comprising the amino acid sequences of SEQ ID NOs: 47, 4, and 48, respectively. In certain embodiments, the VL comprises CDR1, CDR2, and CDR3 comprising the amino acid sequences of SEQ ID NOs: 49, 7, and 8, respectively.

In certain embodiments, the antigen-binding site of the present disclosure is derived from AB1018 (11F09-VH BM2-VK BM1 M33L). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 49, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 104.

In certain embodiments, the VH comprises a CDR1, CDR2, and CDR3 comprising the amino acid sequences of SEQ ID NOs: 47, 4, and 48, respectively. In certain embodiments, the VL comprises a CDR1, CDR2, and CDR3 comprising the amino acid sequences of SEQ ID NOs: 92, 7, and 8, respectively.

In certain embodiments, the antigen-binding site of the present disclosure is derived from AB1019 (11F09-VH BM2-VK BM1 M33V). In certain embodiments, the antigen-binding site comprises (a) a VH that comprises a CDR1, CDR2, and CDR3 comprising the amino acid sequences of SEQ ID NOs: 47, 4, and 48, respectively; and (b) a VL that comprises a CDR1, CDR2, and CDR3 comprising the amino acid sequences of SEQ ID NOs: 92, 7, and 8, respectively.

For example, in certain embodiments, the antigen-binding site of the present disclosure comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91.

In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 105.

In each of the foregoing embodiments, it is contemplated herein that the VH and/or VL sequences that together bind 5T4 may contain amino acid alterations (e.g., at least 1, 2, 3, 4, 5, or 10 amino acid substitutions, deletions, or additions) in the framework regions of the VH and/or VL without affecting their ability to bind to 5T4 significantly.

In certain embodiments, the antigen-binding site of the present disclosure binds human 5T4 with a K_{D} (i.e., dissociation constant) of 1 nM or lower, 5 nM or lower, 10 nM or lower, 15 nM or lower, or 20 nM or lower, as measured by surface plasmon resonance (SPR) (e.g., using the method described in Example 1 infra) or by bio-layer interferometry (BLI), and/or binds 5T4 from a body fluid, tissue, and/or cell of a subject. In certain embodiments, an antigen-binding site of the present disclosure has a K_{d} (i.e., off-rate, also called K_{off}) equal to or lower than 1 × 10⁻⁵, 1 × 10⁻⁴, 1 × 10⁻³, 5 × 10⁻³, 0.01, 0.02, or 0.05 1/s, as measured by SPR (e.g., using the method described in Example 1 infra) or by BLI.

In certain embodiments, the antigen-binding site of the present disclosure binds cynomolgus 5T4 with a K_{D} (i.e., dissociation constant) of 5 nM or lower, 10 nM or lower, 15 nM or lower, 20 nM or lower, or 30 nM or lower, as measured by surface plasmon resonance (SPR) (e.g., using the method described in Example 1 infra) or by bio-layer interferometry (BLI), and/or binds 5T4 from a body fluid, tissue, and/or cell of a subject. In certain embodiments, an antigen-binding site of the present disclosure has a K_{d} (i.e., off-rate, also called K_{Off}) equal to or lower than 1 × 10⁻³, 5 × 10⁻³, 0.01, 0.02, or 0.03 1/s, as measured by SPR (e.g., using the method described in Example 1 infra) or by BLI.

In another aspect, the present disclosure provides an antigen-binding site that comprises a VH comprising a CDR1, CDR2, and CDR3 comprising the amino acid sequences of SEQ ID NOs:3, 4, and 5, respectively, and a VL comprising a CDR1, CDR2, and CDR3 comprising the amino acid sequences of SEQ ID NOs:6, 7, and 8, respectively, wherein the antigen-binding site binds 5T4 within the LRR1 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising a CDR1, CDR2, and CDR3 comprising the amino acid sequences of SEQ ID NOs:3, 4, and 5, respectively, and a VL comprising a CDR1, CDR2, and CDR3 comprising the amino acid sequences of SEQ ID NOs:6, 7, and 8, respectively, is a human antigen-binding site. In some embodiments, the antigen-binding site that comprises a VH comprising a CDR1, CDR2, and CDR3 comprising the amino acid sequences of SEQ ID NOs:3, 4, and 5, respectively, and a VL comprising a CDR1, CDR2, and CDR3 comprising the amino acid sequences of SEQ ID NOs:6, 7, and 8, respectively, is a murine antigen-binding site.

In another aspect, the present disclosure provides an antigen-binding site that comprises a VH comprising a CDR1, CDR2, and CDR3 comprising the amino acid sequences of SEQ ID NOs:47, 4, and 48, respectively, and a VL comprising a CDR1, CDR2, and CDR3 comprising the amino acid sequences of SEQ ID NOs:50, 7, and 8, respectively, wherein the antigen-binding site binds 5T4 within the LRR1 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising a CDR1, CDR2, and CDR3 comprising the amino acid sequences of SEQ ID NOs: 47, 4, and 48, respectively, and a VL comprising a CDR1, CDR2, and CDR3 comprising the amino acid sequences of SEQ ID NOs:50, 7, and 8, respectively, is a human antigen-binding site. In some embodiments, the antigen-binding site that comprises a VH comprising a CDR1, CDR2, and CDR3 comprising the amino acid sequences of SEQ ID NOs: 47, 4, and 48, respectively, and a VL comprising a CDR1, CDR2, and CDR3 comprising the amino acid sequences of SEQ ID NOs:50, 7, and 8, respectively, is a murine antigen-binding site.

In another aspect, the present disclosure provides an antigen-binding site that comprises a VH comprising a CDR1, CDR2, and CDR3 comprising the amino acid sequences of SEQ ID NOs:53, 54, and 55, respectively, and a VL comprising a CDR1, CDR2, and CDR3 comprising the amino acid sequences of SEQ ID NOs: 56, 57, and 8, respectively, wherein the antigen-binding site binds 5T4 within the LRR2 domain. CDR sequences are recognized as features driving antigen-binding properties, accordingly, one of skill in the art understands that an antigen-binding site comprising the same CDRs is expected to exhibit similar antigen-binding properties. In some embodiments, the antigen-binding site that comprises a VH comprising a CDR1, CDR2, and CDR3 comprising the amino acid sequences of SEQ ID NOs:53, 54, and 55, respectively, and a VL comprising a CDR1, CDR2, and CDR3 comprising the amino acid sequences of SEQ ID NOs:56, 57, and 8, respectively, is a human antigen-binding site. In some embodiments, the antigen-binding site that comprises a VH comprising a CDR1, CDR2, and CDR3 comprising the amino acid sequences of SEQ ID NOs:53, 54, and 55, respectively, and a VL comprising a CDR1, CDR2, and CDR3 comprising the amino acid sequences of SEQ ID NOs:56, 57, and 8, respectively, is a murine antigen-binding site.

In another aspect, the present disclosure provides an antigen-binding site that competes for binding to 5T4 (e.g., human 5T4) with an antigen-binding site described above. In certain embodiments, the antigen-binding site of the present disclosure competes with an antigen-binding site derived from AB1002, 05H04, 10F10 21*05, 10F10 23*03, 10F10 48*01, 10F10 11*01, 10F10 21*05, 11F09 48*01, 11F09 21*05, 11F09 11*01, 11F09 23*03, or the scFV and humanized versions derived therefrom and disclosed above, for binding to 5T4. In certain embodiments, the antigen-binding site of the present disclosure competes with an antigen-binding site derived from AB1002, 05H04, 10F10 21*05, 10F10 23*03, 10F10 48*01, 10F10 11*01, 10F10 21*05, 11F09 48*01, 11F09 21*05, 11F09 11*01, 11F09 23*03, 08E06, 08E06-humanized variant 1, 08E06-humanized variant 2, AB1011, AB1012, AB1013, AB1014, AB1015, AB1016, AB1017, AB1018, AB1019, or the scFV and humanized versions derived therefrom and disclosed above for binding to 5T4. In some embodiments, the antigen-binding site competes with AB 1002 for binding to 5T4.

In another aspect, the present disclosure provides an antigen-binding site that comprises a VH comprising a CDR1, a CDR2, and a CDR3 sequence selected from Table 5 and a VL comprising a CDR1, a CDR2, and a CDR3 sequence comprising the amino acid sequences of SEQ ID NOs: 56, 57, and 8, respectively. For example, in certain embodiments, the antigen-binding site comprises a VH comprising a CDR1, a CDR2, and a CDR3 sequence selected from the group consisting of: (a) GYTFTSY (SEQ ID NO:53), DSSDSK (SEQ ID NO:54), and GGYLWFAY (SEQ ID NO:55); (b) GYTFGSY (SEQ ID NO:73), DASTEK (SEQ ID NO:74), and GGYLWFQY (SEQ ID NO:75); (c) GYLFTSY (SEQ ID NO:76), SVSDAK (SEQ ID NO:77), and GGYLWFKY (SEQ ID NO:78); (d) GYTFGSY (SEQ ID NO:73), DARSAK (SEQ ID NO:79), and GGYLWFKY(SEQ ID NO:78); (e) GYRFTSY (SEQ ID NO:80), DASSAK (SEQ ID NO:81), and GGYLWFKY (SEQ ID NO:78); (f) GYGFTSY (SEQ ID NO:82), DARTAK (SEQ ID NO:83), and GGYLWYAY (SEQ ID NO:84); (g) GYTFTSY (SEQ ID NO:53), DASDAK (SEQ ID NO:85), and GGYLWYHY (SEQ ID NO:86); (h) GYTFTSY (SEQ ID NO:53), DASDAK (SEQ ID NO:85), and GGYLWYSY (SEQ ID NO:87); (i) GYTFTSY (SEQ ID NO:53), DASDAK (SEQ ID NO:85), and GGYLWYAY (SEQ ID NO:84); (j) GYSFTSY (SEQ ID NO:88), DASDAK (SEQ ID NO:85), and GGYLWFKY (SEQ ID NO:78); (k) GYTFTSY (SEQ ID NO:53), DASDAK (SEQ ID NO:85), and GGYLWFKY (SEQ ID NO:78); and (1) GYGFTSY (SEQ ID NO:82), DARTAK (SEQ ID NO:83), and GGHLWYAY (SEQ ID NO:89). Such antigen-binding site that binds to 5T4 can be formed by combining any one of these VHs with a VL comprising a CDR1, a CDR2, and a CDR3 sequence comprising the amino acid sequences of SEQ ID NOs: 56, 57, and 8, respectively.

The present disclosure provides an isolated nucleic acid encoding any one of the antigen-binding sites or proteins described herein. The present disclosure also provides for nucleic acids encoding any 5T4 antigen-binding site as described herein. Accordingly, the present disclosure provides for nucleic acids encoding one or more of the chains comprising an antigen-binding site or protein as described herein. In some embodiments, the nucleic acid encodes a VH that comprises an amino acid sequence that is at least 90% (e.g., at least 91%, at least 92%, at least 93%, at least 94%, at least 95%, at least 96%, at least 97%, at least 98%, at least 99%, or 100%) identical to the VH of an antibody disclosed in Table 1. In some embodiments, the nucleic acid encodes a VL that comprises an amino acid sequence that is at least 90% (e.g., at least 91%, at least 92%, at least 93%, at least 94%, at least 95%, at least 96%, at least 97%, at least 98%, at least 99%, or 100%) identical to the VL of the same antibody disclosed in Table 1. In certain embodiments, the nucleic acid encodes a VH comprising a CDR1 sequence comprising SEQ ID NO:3, a CDR2 sequence comprising SEQ ID NO:4, and a CDR3 sequence comprising SEQ ID NO:5. In certain embodiments, the nucleic acid encodes a VL comprising a CDR1 sequence comprising SEQ ID NO:6, a CDR2 sequence comprising SEQ ID NO:7, and a CDR3 sequence comprising SEQ ID NO:8. In certain embodiments, the nucleic acid encodes a VH comprising a CDR1 sequence comprising SEQ ID NO:3, a CDR2 sequence comprising SEQ ID NO:4, and a CDR3 sequence comprising SEQ ID NO:5, and a VL comprising a CDR1 sequence comprising SEQ ID NO:6, a CDR2 sequence comprising SEQ ID NO:7, and a CDR3 sequence comprising SEQ ID NO:8. In certain embodiments, the nucleic acid encodes a VH comprising the amino acid sequence of SEQ ID NO:9. In certain embodiments, the nucleic acid encodes a VL comprising the amino acid sequence of SEQ ID NO: 10. In certain embodiments, the nucleic acid encodes a VH comprising the amino acid sequence of SEQ ID NO: 11. In certain embodiments, the nucleic acid encodes a VL comprising the amino acid sequence of SEQ ID NO: 12. The present disclosure still further provides for nucleic acids encoding an Fc domain or portion thereof as described herein.

Exemplary nucleic acid sequences of the present disclosure are provided in Table 2. AB1310, as used herein, refers to a multi specific binding protein comprising the AB 1002 scFv (VL-VH). AB 1783, as used herein, refers to a multispecific binding protein comprising the AB 1002 scFv (VL-VH). The amino acid sequences of AB 1310 and AB 1783 are identical. The nucleic acid sequences, as shown in Table 2, are distinct for expression in different host cells. AB 1783 was optimized for expression in CHO cells.

TABLE 2

Sequences of Exemplary Nucleic Acid Sequences Encoding Antigen-Binding Sites that Bind 5T4

| | |
|---|---|
| Clone | Sequence |
| AB1310 5T4-scFv-Fc | GTTAGGCGTTTTGCGCTGCTTCGCGATGTACGGGCCAGATATACGCGTTGACATT GATTATTGACTAGTTATTAATAGTAATCAATTACGGGGTCATTAGTTCATAGCCC ATATATGGAGTTCCGCGTTACATAACTTACGGTAAATGGCCCGCCTGGCTGACCG CCCAACGACCCCCGCCCATTGACGTCAATAATGACGTATGTTCCCATAGTAACGC CAATAGGGACTTTCCATTGACGTCAATGGGTGGAGTATTTACGGTAAACTGCCCA CTTGGCAGTACATCAAGTGTATCATATGCCAAGTACGCCCCCTATTGACGTCAAT GACGGTAAATGGCCCGCCTGGCATTATGCCCAGTACATGACCTTATGGGACTTTC CTACTTGGCAGTACATCTACGTATTAGTCATCGCTATTACCATGGTGATGCGGTT TTGGCAGTACATCAATGGGCGTGGATAGCGGTTTGACTCACGGGGATTTCCAAGT CTCCACCCCATTGACGTCAATGGGAGTTTGTTTTGGCACCAAAATCAACGGGACT TTCCAAAATGTCGTAACAACTCCGCCCCATTGACGCAAATGGGCGGTAGGCGTGT ACGGTGGGAGGTCTATATAAGCAGAGCTCGTTTAGTGAACCGTCAGATCGCCTGG AGACGCCATCCACGCTGTTTTGACCTCCATAGAAGACACCGGGACCGATCCAGCC TCCGGACTCTAGAGGATCGAACCCTTAAGCTTGCCACCATGGACATGAGAGTGCC CGCTCAACTGCTGGGACTGCTGCTGCTTTGGCTTCCTGGCGCTAGATGCGACATC CAGCTGACACAGAGCCCTAGCAGCCTGTCTGCCTCTGTGGGCGACAGAGTGACCA TCACCTGTAGAGCCAGCCAGAGCGTGACCACCAGCAACTACAACTACATGCACTG |
| | GTTCCAGCAGAAGCCCGGCAAGGCCCCTAAGCTGCTGATCAAGTTCGCCAGCAAC CTGGAAAGCGGCGTGCCCAGCAGATTTTCTGGCAGCGGCTCTGGCACCGACTTCA CCCTGACCATATCTAGCCTGCAGCCTGAGGACTTCGCCACCTACTACTGTCAGCA CAGCTGGGAGATCCCTTGGACCTTTGGCTGTGGCACCAAGGTGGAAATCAAAGGC GGCGGAGGATCTGGCGGAGGTGGAAGCGGAGGCGGAGGAAGCGGTGGCGGCGGAT CTGAAGTTCAGCTGGTTGAAAGTGGCGGAGGCCTGGTTAAGCCTGGCGGATCTCT GAGACTGAGCTGTGCCGCCTCCAGATTCACCTTCAGCGACTTCGGAATGCACTGG GTCCGACAGGCCCCTGGCAAATGTCTGGAATGGGTGTCCTACATCAGCAGCGGCA GCAGCACCATCTACTACGCCGATAGCGTGAAGGGCAGATTCACAATCAGCCGGGA CAACGCCAAGAACAGCCTGTACCTGCAGATGAACTCCCTGAGAGCCGAGGACACC GCCGTGTACTATTGTGCCAGCAGCCAGTCCTACTACCGGGGCACACTGGATTATT GGGGCCAGGGCACAACCGTGACAGTGTCCAGCGCCAGCGATAAGACCCACACCTG TCCTCCATGTCCTGCTCCAGAACTGCTCGGCGGACCTTCCGTGTTCCTGTTTCCT CCAAAGCCTAAGGACACCCTGATGATCTCCAGAACACCCGAAGTGACCTGCGTGG TGGTGGATGTGTCTCACGAGGACCCTGAAGTGAAGTTCAATTGGTACGTGGACGG CGTGGAAGTGCACAATGCCAAGACCAAGCCTAGAGAGGAACAGTACAACAGCACC TACAGAGTGGTGTCCGTGCTGACCGTGCTGCACCAGGATTGGCTGAACGGCAAAG AGTACAAGTGCAAGGTGTCCAACAAGGCCCTGCCTGCTCCTATCGAGAAAACCAT CAGCAAGGCCAAGGGCCAGCCTCGCGAGCCTAGAGTGTATACCTTGCCTCCATGC CGGGACGAGCTGACCAAGAATCAGGTGTCCCTGACCTGCCTGGTCAAGGGCTTCT ACCCTTCCGATATCGCCGTGGAATGGGAGAGCAATGGCCAGCCTGAGAACAACTA CAAGACCACACCTCCTGTGCTGGTGTCCGACGGCAGCTTTACCCTGTACAGCAAG CTGACAGTGGACAAGAGCAGATGGCAGCAGGGCAACGTGTTCAGCTGCAGCGTGA TGCACGAGGCCCTGCACAACCACTACACCCAGAAGTCCCTGAGCCTGTCTCCTGG ATGATAATCTAGAAAGGGTTCGATCCCTACCGGTTAGTAATGAGTTTGATATCTC GACAATCAACCTCTGGATTACAAAATTTGTGAAAGATTGACTGGTATTCTTAACT ATGTTGCTCCTTTTACGCTATGTGGATACGCTGCTTTAATGCCTTTGTATCATGC TATTGCTTCCCGTATGGCTTTCATTTTCTCCTCCTTGTATAAATCCTGGTTGCTG TCTCTTTATGAGGAGTTGTGGCCCGTTGTCAGGCAACGTGGCGTGGTGTGCACTG TGTTTGCTGACGCAACCCCCACTGGTTGGGGCATTGCCACCACCTGTCAGCTCCT TTCCGGGACTTTCGCTTTCCCCCTCCCTATTGCCACGGCGGAACTCATCGCCGCC TGCCTTGCCCGCTGCTGGACAGGGGCTCGGCTGTTGGGCACTGACAATTCCGTGG TGTTGTCGGGGAAGCTGACGTCCTTTCCATGGCTGCTCGCCTGTGTTGCCACCTG GATTCTGCGCGGGACGTCCTTCTGCTACGTCCCTTCGGCCCTCAATCCAGCGGAC CTTCCTTCCCGCGGCCTGCTGCCGGCTCTGCGGCCTCTTCCGCGTCTTCGCCTTC GCCCTCAGACGAGTCGGATCTCCCTTTGGGCCGCCTCCCCGCCTGGAACGGGGGA GGCTAACTGAAACACGGAAGGAGACAATACCGGAAGGAACCCGCGCTATGACGGC AATAAAAAGACAGAATAAAACGCACGGGTGTTGGGTCGTTTGTTCATAAACGCGG GGTTCGGTCCCAGGGCTGGCACTCTGTCGATACCCCACCGAGACCCCATTGGGGC CAATACGCCCGCGTTTCTTCCTTTTCCCCACCCCACCCCCCAAGTTCGGGTGAAG GCCCAGGGCTCGCAGCCAACGTCGGGGCGGCAGGCCCTGCCATAGCAGATCTGCG CAGCTGGGGCTCTAGGGGGTATCCCCACGCGCCCTGTAGCGGCGCATTAAGCGCG GCGGGTGTGGTGGTTACGCGCAGCGTGACCGCTACACTTGCCAGCGCCCTAGCGC CCGCTCCTTTCGCTTTCTTCCCTTCCTTTCTCGCCACGTTCGCCGGCTTTCCCCG TCAAGCTCTAAATCGGGGGCTCCCTTTAGGGTTCCGATTTAGTGCTTTACGGCAC CTCGACCCCAAAAAACTTGATTAGGGTGATGGTTCACGTAGTGGGCCATCGCCCT GATAGACGGTTTTTCGCCCTTTGACGTTGGAGTCCACGTTCTTTAATAGTGGACT CTTGTTCCAAACTGGAACAACACTCAACCCTATCTCGGTCTATTCTTTTGATTTA TAAGGGATTTTGCCGATTTCGGCCTATTGGTTAAAAAATGAGCTGATTTAACAAA AATTTAACGCGAATTAATTCTGTGGAATGTGTGTCAGTTAGGGTGTGGAAAGTCC CCAGGCTCCCCAGCAGGCAGAAGTATGCAAAGCATGCATCTCAATTAGTCAGCAA |
| | CCAGGTGTGGAAAGTCCCCAGGCTCCCCAGCAGGCAGAAGTATGCAAAGCATGCA TCTCAATTAGTCAGCAACCATAGTCCCGCCCCTAACTCCGCCCATCCCGCCCCTA ACTCCGCCCAGTTCCGCCCATTCTCCGCCCCATGGCTGACTAATTTTTTTTATTT ATGCAGAGGCCGAGGCCGCCTCTGCCTCTGAGCTATTCCAGAAGTAGTGAGGAGG CTTTTTTGGAGGCCTAGGCTTTTGCAAAAAGCTCCCGGGAGCTTGTATATCCATT TTCGGATCTGATCAAGAGACAGGATGAGGATCGTTTCGCATGATTGAACAAGATG GATTGCACGCAGGTTCTCCGGCCGCTTGGGTGGAGAGGCTATTCGGCTATGACTG GGCACAACAGACAATCGGCTGCTCTGATGCCGCCGTGTTCCGGCTGTCAGCGCAG GGGCGCCCGGTTCTTTTTGTCAAGACCGACCTGTCCGGTGCCCTGAATGAACTGC AGGACGAGGCAGCGCGGCTATCGTGGCTGGCCACGACGGGCGTTCCTTGCGCAGC TGTGCTCGACGTTGTCACTGAAGCGGGAAGGGACTGGCTGCTATTGGGCGAAGTG CCGGGGCAGGATCTCCTGTCATCTCACCTTGCTCCTGCCGAGAAAGTATCCATCA TGGCTGATGCAATGCGGCGGCTGCATACGCTTGATCCGGCTACCTGCCCATTCGA CCACCAAGCGAAACATCGCATCGAGCGAGCACGTACTCGGATGGAAGCCGGTCTT GTCGATCAGGATGATCTGGACGAAGAGCATCAGGGGCTCGCGCCAGCCGAACTGT TCGCCAGGCTCAAGGCGCGCATGCCCGACGGCGAGGATCTCGTCGTGACCCATGG CGATGCCTGCTTGCCGAATATCATGGTGGAAAATGGCCGCTTTTCTGGATTCATC GACTGTGGCCGGCTGGGTGTGGCGGACCGCTATCAGGACATAGCGTTGGCTACCC GTGATATTGCTGAAGAGCTTGGCGGCGAATGGGCTGACCGCTTCCTCGTGCTTTA CGGTATCGCCGCTCCCGATTCGCAGCGCATCGCCTTCTATCGCCTTCTTGACGAG TTCTTCTGAGCGGGACTCTGGGGTTCGCGAAATGACCGACCAAGCGACGCCCAAC CTGCCATCACGAGATTTCGATTCCACCGCCGCCTTCTATGAAAGGTTGGGCTTCG GAATCGTTTTCCGGGACGCCGGCTGGATGATCCTCCAGCGCGGGGATCTCATGCT GGAGTTCTTCGCCCACCCCAACTTGTTTATTGCAGCTTATAATGGTTACAAATAA AGCAATAGCATCACAAATTTCACAAATAAAGCATTTTTTTCACTGCATTCTAGTT GTGGTTTGTCCAAACTCATCAATGTATCTTATCATGTCTGTATACCGTCGACCTC TAGCTAGAGCTTGGCGTAATCATGGTCATAGCTGTTTCCTGTGTGAAATTGTTAT CCGCTCACAATTCCACACAACATACGAGCCGGAAGCATAAAGTGTAAAGCCTGGG GTGCCTAATGAGTGAGCTAACTCACATTAATTGCGTTGCGCTCACTGCCCGCTTT CCAGTCGGGAAACCTGTCGTGCCAGCTGCATTAATGAATCGGCCAACGCGCGGGG AGAGGCGGTTTGCGTATTGGGCGCTCTTCCGCTTCCTCGCTCACTGACTCGCTGC GCTCGGTCGTTCGGCTGCGGCGAGCGGTATCAGCTCACTCAAAGGCGGTAATACG GTTATCCACAGAATCAGGGGATAACGCAGGAAAGAACATGTGAGCAAAAGGCCAG CAAAAGGCCAGGAACCGTAAAAAGGCCGCGTTGCTGGCGTTTTTCCATAGGCTCC GCCCCCCTGACGAGCATCACAAAAATCGACGCTCAAGTCAGAGGTGGCGAAACCC GACAGGACTATAAAGATACCAGGCGTTTCCCCCTGGAAGCTCCCTCGTGCGCTCT CCTGTTCCGACCCTGCCGCTTACCGGATACCTGTCCGCCTTTCTCCCTTCGGGAA GCGTGGCGCTTTCTCATAGCTCACGCTGTAGGTATCTCAGTTCGGTGTAGGTCGT TCGCTCCAAGCTGGGCTGTGTGCACGAACCCCCCGTTCAGCCCGACCGCTGCGCC TTATCCGGTAACTATCGTCTTGAGTCCAACCCGGTAAGACACGACTTATCGCCAC TGGCAGCAGCCACTGGTAACAGGATTAGCAGAGCGAGGTATGTAGGCGGTGCTAC AGAGTTCTTGAAGTGGTGGCCTAACTACGGCTACACTAGAAGAACAGTATTTGGT ATCTGCGCTCTGCTGAAGCCAGTTACCTTCGGAAAAAGAGTTGGTAGCTCTTGAT CCGGCAAACAAACCACCGCTGGTAGCGGTGGTTTTTTTGTTTGCAAGCAGCAGAT TACGCGCAGAAAAAAAGGATCTCAAGAAGATCCTTTGATCTTTTCTACGGGGTCT GACGCTCAGTGGAACGAAAACTCACGTTAAGGGATTTTGGTCATGAGATTATCAA AAAGGATCTTCACCTAGATCCTTTTAAATTAAAAATGAAGTTTTAAATCAATCTA AAGTATATATGAGTAAACTTGGTCTGACAGTTACCAATGCTTAATCAGTGAGGCA CCTATCTCAGCGATCTGTCTATTTCGTTCATCCATAGTTGCCTGACTCCCCGTCG TGTAGATAACTACGATACGGGAGGGCTTACCATCTGGCCCCAGTGCTGCAATGAT ACCGCGAGACCCACGCTCACCGGCTCCAGATTTATCAGCAATAAACCAGCCAGCC |
| | GGAAGGGCCGAGCGCAGAAGTGGTCCTGCAACTTTATCCGCCTCCATCCAGTCTA TTAATTGTTGCCGGGAAGCTAGAGTAAGTAGTTCGCCAGTTAATAGTTTGCGCAA CGTTGTTGCCATTGCTACAGGCATCGTGGTGTCACGCTCGTCGTTTGGTATGGCT TCATTCAGCTCCGGTTCCCAACGATCAAGGCGAGTTACATGATCCCCCATGTTGT GCAAAAAAGCGGTTAGCTCCTTCGGTCCTCCGATCGTTGTCAGAAGTAAGTTGGC CGCAGTGTTATCACTCATGGTTATGGCAGCACTGCATAATTCTCTTACTGTCATG CCATCCGTAAGATGCTTTTCTGTGACTGGTGAGTACTCAACCAAGTCATTCTGAG AATAGTGTATGCGGCGACCGAGTTGCTCTTGCCCGGCGTCAATACGGGATAATAC CGCGCCACATAGCAGAACTTTAAAAGTGCTCATCATTGGAAAACGTTCTTCGGGG CGAAAACTCTCAAGGATCTTACCGCTGTTGAGATCCAGTTCGATGTAACCCACTC GTGCACCCAACTGATCTTCAGCATCTTTTACTTTCACCAGCGTTTCTGGGTGAGC AAAAACAGGAAGGCAAAATGCCGCAAAAAAGGGAATAAGGGCGACACGGAAATGT TGAATACTCATACTCTTCCTTTTTCAATATTATTGAAGCATTTATCAGGGTTATT GTCTCATGAGCGGATACATATTTGAATGTATTTAGAAAAATAAACAAATAGGGGT TCCGCGCACATTTCCCCGAAAAGTGCCACCTGACGTCGACGGATCGGGAGATCTC CCGATCCCCTATGGTCGACTCTCAGTACAATCTGCTCTGATGCCGCATAGTTAAG CCAGTATCTGCTCCCTGCTTGTGTGTTGGAGGTCGCTGAGTAGTGCGCGAGCAAA ATTTAAGCTACAACAAGGCAAGGCTTGACCGACAATTGCATGAAGAATCTGCTTA GG (SEQ ID NO:109) |
| AB1310 5T4-scFv-Fc | GACATCCAGCTGACACAGAGCCCTAGCAGCCTGTCTGCCTCTGTGGGCGACAGAG TGACCATCACCTGTAGAGCCAGCCAGAGCGTGACCACCAGCAACTACAACTACAT GCACTGGTTCCAGCAGAAGCCCGGCAAGGCCCCTAAGCTGCTGATCAAGTTCGCC AGCAACCTGGAAAGCGGCGTGCCCAGCAGATTTTCTGGCAGCGGCTCTGGCACCG ACTTCACCCTGACCATATCTAGCCTGCAGCCTGAGGACTTCGCCACCTACTACTG TCAGCACAGCTGGGAGATCCCTTGGACCTTTGGCTGTGGCACCAAGGTGGAAATC AAAGGCGGCGGAGGATCTGGCGGAGGTGGAAGCGGAGGCGGAGGAAGCGGTGGCG GCGGATCTGAAGTTCAGCTGGTTGAAAGTGGCGGAGGCCTGGTTAAGCCTGGCGG ATCTCTGAGACTGAGCTGTGCCGCCTCCAGATTCACCTTCAGCGACTTCGGAATG CACTGGGTCCGACAGGCCCCTGGCAAATGTCTGGAATGGGTGTCCTACATCAGCA GCGGCAGCAGCACCATCTACTACGCCGATAGCGTGAAGGGCAGATTCACAATCAG CCGGGACAACGCCAAGAACAGCCTGTACCTGCAGATGAACTCCCTGAGAGCCGAG GACACCGCCGTGTACTATTGTGCCAGCAGCCAGTCCTACTACCGGGGCACACTGG ATTATTGGGGCCAGGGCACAACCGTGACAGTGTCCAGCGCCAGCGATAAGACCCA CACCTGTCCTCCATGTCCTGCTCCAGAACTGCTCGGCGGACCTTCCGTGTTCCTG TTTCCTCCAAAGCCTAAGGACACCCTGATGATCTCCAGAACACCCGAAGTGACCT GCGTGGTGGTGGATGTGTCTCACGAGGACCCTGAAGTGAAGTTCAATTGGTACGT GGACGGCGTGGAAGTGCACAATGCCAAGACCAAGCCTAGAGAGGAACAGTACAAC AGCACCTACAGAGTGGTGTCCGTGCTGACCGTGCTGCACCAGGATTGGCTGAACG GCAAAGAGTACAAGTGCAAGGTGTCCAACAAGGCCCTGCCTGCTCCTATCGAGAA AACCATCAGCAAGGCCAAGGGCCAGCCTCGCGAGCCTAGAGTGTATACCTTGCCT CCATGCCGGGACGAGCTGACCAAGAATCAGGTGTCCCTGACCTGCCTGGTCAAGG GCTTCTACCCTTCCGATATCGCCGTGGAATGGGAGAGCAATGGCCAGCCTGAGAA CAACTACAAGACCACACCTCCTGTGCTGGTGTCCGACGGCAGCTTTACCCTGTAC AGCAAGCTGACAGTGGACAAGAGCAGATGGCAGCAGGGCAACGTGTTCAGCTGCA GCGTGATGCACGAGGCCCTGCACAACCACTACACCCAGAAGTCCCTGAGCCTGTC TCCTGGA (SEQ ID NO: 241) |
| AB 1783 5T4-scFv-Fc | AAGCTTACCGCCACCATGGACATGCGCGTCCCCGCGCAGCTCCTCGGCCTCCTGC TGCTCTGGCTCCCCGGCGCCCGCTGCGACATCCAACTGACCCAGTCCCCGTCCTC CCTGAGCGCATCTGTCGGCGACCGGGTGACCATCACCTGCCGGGCCTCGCAGTCC GTGACCACGTCGAACTACAACTACATGCACTGGTTCCAGCAGAAGCCCGGTAAGG CGCCCAAGCTGCTCATCAAGTTCGCGTCCAACCTGGAGTCGGGCGTGCCGTCGCG GTTCTCCGGCTCCGGCAGCGGCACCGACTTCACGCTGACGATCTCGTCCCTCCAG CCCGAGGACTTCGCCACGTACTACTGCCAGCACTCCTGGGAGATCCCGTGGACGT TCGGGTGCGGCACCAAGGTCGAGATCAAGGGAGGCGGGGGCAGTGGGGGCGGAGG CTCTGGGGGCGGCGGCAGTGGCGGCGGCGGCTCGGAGGTCCAGCTGGTAGAGTCC GGAGGCGGCCTCGTGAAGCCGGGCGGCTCCCTGCGCCTGTCCTGCGCGGCCAGCC GTTTCACCTTCTCGGACTTCGGGATGCATTGGGTGCGCCAGGCCCCAGGCAAGTG CCTGGAGTGGGTTTCCTACATCTCGTCCGGCTCGAGTACCATCTATTACGCCGAC AGCGTCAAGGGACGCTTCACGATCTCGCGGGACAACGCCAAGAACAGCCTTTACC TGCAGATGAACAGCCTGCGCGCGGAGGACACCGCCGTCTACTACTGCGCCTCGTC CCAGAGCTATTACCGGGGTACCCTCGACTACTGGGGCCAGGGGACGACGGTGACC GTCAGCTCCGCCAGCGACAAGACCCACACGTGCCCCCCGTGCCCGGCCCCGGAGC TGCTGGGAGGCCCGTCCGTGTTCCTGTTCCCCCCGAAGCCCAAGGACACCCTGAT GATCTCGCGCACCCCGGAGGTGACCTGCGTAGTCGTCGACGTGTCCCACGAGGAC CCCGAGGTGAAGTTCAACTGGTACGTGGACGGCGTCGAGGTCCACAACGCGAAGA CCAAGCCGCGCGAGGAGCAGTACAACAGCACCTACCGGGTCGTGAGCGTCCTGAC CGTGCTGCACCAAGACTGGCTCAACGGTAAGGAGTACAAGTGTAAGGTGAGCAAC AAGGCCCTCCCTGCCCCGATTGAGAAGACCATCTCGAAGGCTAAGGGCCAGCCGC GGGAGCCCCGGGTGTACACGCTCCCGCCCTGCCGGGACGAACTGACCAAAAACCA GGTGTCCCTGACGTGTCTGGTGAAGGGCTTCTACCCCTCCGACATCGCCGTCGAG TGGGAGTCCAATGGTCAGCCCGAGAACAACTACAAGACGACCCCGCCCGTGCTGG TGAGCGACGGGTCCTTCACCCTGTATTCCAAGCTCACGGTCGACAAGTCCCGCTG GCAGCAGGGCAACGTCTTCTCGTGCTCCGTGATGCACGAGGCCCTCCACAACCAC TACACCCAGAAGTCACTGTCACTCTCCCCCGGCTGATAATCTAGA (SEQ ID NO:110) |
| AB 1783 5T4-scFv-Fc | GACATCCAACTGACCCAGTCCCCGTCCTCCCTGAGCGCATCTGTCGGCGACCGGG TGACCATCACCTGCCGGGCCTCGCAGTCCGTGACCACGTCGAACTACAACTACAT GCACTGGTTCCAGCAGAAGCCCGGTAAGGCGCCCAAGCTGCTCATCAAGTTCGCG TCCAACCTGGAGTCGGGCGTGCCGTCGCGGTTCTCCGGCTCCGGCAGCGGCACCG ACTTCACGCTGACGATCTCGTCCCTCCAGCCCGAGGACTTCGCCACGTACTACTG CCAGCACTCCTGGGAGATCCCGTGGACGTTCGGGTGCGGCACCAAGGTCGAGATC AAGGGAGGCGGGGGCAGTGGGGGCGGAGGCTCTGGGGGCGGCGGCAGTGGCGGCG GCGGCTCGGAGGTCCAGCTGGTAGAGTCCGGAGGCGGCCTCGTGAAGCCGGGCGG CTCCCTGCGCCTGTCCTGCGCGGCCAGCCGTTTCACCTTCTCGGACTTCGGGATG CATTGGGTGCGCCAGGCCCCAGGCAAGTGCCTGGAGTGGGTTTCCTACATCTCGT CCGGCTCGAGTACCATCTATTACGCCGACAGCGTCAAGGGACGCTTCACGATCTC GCGGGACAACGCCAAGAACAGCCTTTACCTGCAGATGAACAGCCTGCGCGCGGAG GACACCGCCGTCTACTACTGCGCCTCGTCCCAGAGCTATTACCGGGGTACCCTCG ACTACTGGGGCCAGGGGACGACGGTGACCGTCAGCTCCGCCAGCGACAAGACCCA CACGTGCCCCCCGTGCCCGGCCCCGGAGCTGCTGGGAGGCCCGTCCGTGTTCCTG TTCCCCCCGAAGCCCAAGGACACCCTGATGATCTCGCGCACCCCGGAGGTGACCT GCGTAGTCGTCGACGTGTCCCACGAGGACCCCGAGGTGAAGTTCAACTGGTACGT GGACGGCGTCGAGGTCCACAACGCGAAGACCAAGCCGCGCGAGGAGCAGTACAAC AGCACCTACCGGGTCGTGAGCGTCCTGACCGTGCTGCACCAAGACTGGCTCAACG GTAAGGAGTACAAGTGTAAGGTGAGCAACAAGGCCCTCCCTGCCCCGATTGAGAA GACCATCTCGAAGGCTAAGGGCCAGCCGCGGGAGCCCCGGGTGTACACGCTCCCG CCCTGCCGGGACGAACTGACCAAAAACCAGGTGTCCCTGACGTGTCTGGTGAAGG |
| | GCTTCTACCCCTCCGACATCGCCGTCGAGTGGGAGTCCAATGGTCAGCCCGAGAA CAACTACAAGACGACCCCGCCCGTGCTGGTGAGCGACGGGTCCTTCACCCTGTAT TCCAAGCTCACGGTCGACAAGTCCCGCTGGCAGCAGGGCAACGTCTTCTCGTGCT CCGTGATGCACGAGGCCCTCCACAACCACTACACCCAGAAGTCACTGTCACTCTC CCCCGGC (SEQ ID NO: 242) |

In certain embodiments, a nucleic acid molecule of the present disclosure comprises SEQ ID NO: 109. In certain embodiments, a nucleic acid molecule of the present disclosure comprises SEQ ID NO: 110. In certain embodiments, a nucleic acid molecule of the present disclosure comprises SEQ ID NO:241. In certain embodiments, a nucleic acid molecule of the present disclosure comprises SEQ ID NO:242.

### Proteins With Antigen-Binding Sites

An antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)₂ fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain variable fragment or scFv), triomab, knobs-into-holes (kih) IgG with common light chain, crossmab, ortho-Fab IgG, DVD-Ig, 2 in 1-IgG, IgG-scFv, sdFv2-Fc, binanobody, tandAb, dual-affinity retargeting antibody (DART), DART-Fc, scFv-HSA-scFv (where HSA = human serum albumin), or dock-and-lock (DNL)-Fab3. In certain embodiments, an antibody of the present disclosure is an scFv. In certain embodiments, the scFv is in the VH-VL format.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker ((GlyGlyGlyGlySer)₄ (SEQ ID NO: 111)). In some embodiments of the scFv, the heavy chain variable domain is positioned at the N-terminus of the light chain variable domain. In some embodiments of the scFv, the heavy chain variable domain is positioned at the C terminus of the light chain variable domain.

It is contemplated that in an scFv, a VH and a VL can be connected by a linker, e.g., (GlyGlyGlyGlySer)₄ i.e. (G₄S)₄ linker (SEQ ID NO: 111). A skilled person in the art would appreciate that any of the other disclosed linkers (see, e.g., Table 3) may be used in an scFv having a VH and VL sequence disclosed herein (e.g., in Table 1).

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO: 112), (GGS)n (SEQ ID NO: 113), (GGGS)n (SEQ ID NO: 114), (GGSG)n (SEQ ID NO: 115), (GGSGG)ₙ (SEQ ID NO: 116), and (GGGGS)ₙ (SEQ ID NO: 117) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NOs:64-72, 111 and 118, as listed in Table 3.

TABLE 3

| | |
|---|---|
| SEQ ID | Amino Acid Sequence |
| SEQ ID NO:118 | GSGSGSGSGSGSGSGSGSGS |
| SEQ ID NO:64 | GGSGGSGGSGGSGGSGGSGGSGGSGGSGGS |
| SEQ ID NO:65 | GGGSGGGSGGGSGGGSGGGSGGGSGGGSGGGSGGGSGGGS |
| SEQ ID NO:66 | GGSGGGSGGGSGGGSGGGSGGGSGGGSGGGSGGGSGGGSG |
| SEQ ID NO:67 | GGSGGGGSGGGGSGGGGSGGGGSGGGGSGGGGSGGGGSGGGGSG GGGSGG |
| SEQ ID NO:68 | GGGGSGGGGSGGGGSGGGGSGGGGSGGGGSGGGGSGGGGSGGGG SGGGGS |
| SEQ ID NO:111 | GGGGSGGGGSGGGGSGGGGS |
| SEQ ID NO:69 | GGGGSGGGGSGGGGS |
| SEQ ID NO:70 | GGGGSGGGGSGGGGSGGGGSGGGGSGGGGSGGGGSGGGGSGGGG SGGGGSGGGGSGGGGSGGGGSGGGGSGGGGSGGGGSGGGGSGGG GSGGGGSGGGGS |
| SEQ ID NO:71 | GGSGGGGSGGGGSGGGGSGGGGSGGGGSGGGGSGGGGSGGGGSG GGGSGGGGSGGGGSGGGGSGGGGSGGGGSGGGGSGGGGSGGGGS GGGGSGGGGSGG |
| SEQ ID NO:72 | SGSGGGGS |

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, an antigen-binding site disclosed herein can be linked to a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In one embodiment, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence set forth below: DKTHTCPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPG (SEQ ID NO:119) . In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265 - Glu 269, Asn 297 - Thr 299, Ala 327 - Ile 332, Leu 234 - Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S 174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

To facilitate formation of an asymmetric protein, Fc domain heterodimerization is contemplated. Mutations (e.g., amino acid substitutions) in the Fc domain that promote heterodimerization are described, for example, in International Application Publication No. WO2019157366.

The proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third, and fourth expression vectors can be stably transfected together into host cells or chromosomally integrated into the genome of host cells to produce the multimeric proteins.

To achieve the highest yield of the proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of a protein comprising an antigen-binding site disclosed herein. The proteins can be isolated and purified. Such proteins that have been isolated and purified, in some embodiments, are substantially free of at least one component as compared to the multispecific binding protein produced in the culture. Therefore, a purified protein can be partly or completely separated from one or more other substances as it is generated, stored, or subsisted in non-naturally occurring environments. The proteins can be isolated and purified from a cell culture using methods known in the art including centrifugation, depth filtration, cell lysis, homogenization, freeze-thawing, affinity purification, gel filtration, ammonium sulfate or ethanol precipitation, ion exchange chromatography (anion or cation), hydrophobic interaction exchange chromatography, and mixed-mode chromatography. Other well-known methods are described in Process Scale Purification of Antibodies, Second Edition, U. Gottschalk (Ed.), John Wiley & Sons, Inc., Hoboken, NJ (2017). Alternatively, the proteins provided herein can be obtained using well-known recombinant methods (see, for example, Sambrook et al., Molecular Cloning: A Laboratory Manual, Third Ed., Cold Spring Harbor Laboratory, New York (2001); and Ausubel et al., Current Protocols in Molecular Biology, John Wiley & Sons, Baltimore, MD (1999)). The methods and conditions for purification of the proteins provided herein can be chosen by those skilled in the art, and purification monitored, for example, by a binding and/or functional assay as described herein.

Accordingly, in another aspect, the present disclosure provides one or more isolated nucleic acids comprising sequences encoding an immunoglobulin heavy chain and/or immunoglobulin light chain variable region of any one of the foregoing antibodies. The invention provides one or more expression vectors that express the immunoglobulin heavy chain and/or immunoglobulin light chain variable region of any one of the foregoing antibodies. Similarly, the invention provides host cells comprising one or more of the foregoing expression vectors and/or isolated nucleic acids.

In certain embodiments, the antibody of the present disclosure specifically binds 5T4 (e.g., human 5T4 or cynomolgus 5T4) with a K_{D} (i.e., dissociation constant) of 25 nM, 20 nM, 15 nM, 10 nM, 9 nM, 8 nM, 7 nM, 6 nM, 5 nM, 4 nM, 3 nM, 2 nM, 1 nM, 0.1 nM or lower, as measured using standard binding assays, for example, surface plasmon resonance (SPR) (e.g., using the method described in Example 1 infra) or bio-layer interferometry (BLI). In certain embodiments, the antibody as disclosed herein specifically binds 5T4 with a K_{D} less than 9 nM. In certain embodiments, the multispecific binding protein as disclosed herein specifically binds 5T4 with a K_{D} less than 8 nM. In certain embodiments, the antibody as disclosed herein specifically binds 5T4 with a K_{D} less than 7 nM. In certain embodiments, the antibody as disclosed herein specifically binds 5T4 with a K_{D} less than 6 nM. In certain embodiments, the antibody as disclosed herein specifically binds 5T4 with a K_{D} less than 5 nM. In certain embodiments, an antibody of the present disclosure specifically binds 5T4 (e.g., human 5T4 or cynomolgus 5T4) with a K_{d} (i.e., off-rate, also called K_{off}) equal to or lower than 1 × 10⁻⁵, 9 × 10⁻⁴, 8 × 10⁻⁴, 7 × 10⁻⁴, 6 × 10⁻⁴, 5 × 10⁻⁴, 4 × 10⁻⁴, 3 × 10⁻⁴, 2 × 10⁻⁴, 1 × 10⁻⁴, 1 × 10⁻³, 5 × 10⁻³, 0.01, 0.02, or 0.05 1/s, as measured by SPR (e.g., using the method described in Example 1 infra) or by BLI. In certain embodiments the antibody binds 5T4 from a body fluid, tissue and/or cell of a subject.

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human 5T4 protein or fragment thereof) bound to a solid surface or expressed on a cell surface, a test 5T4-binding antibody and a reference antibody. The reference antibody is labeled and the test antibody is unlabeled. Competitive inhibition is measured by determining the amount of labeled reference antibody bound to the solid surface or cells in the presence of the test antibody. Usually, the test antibody is present in excess (e.g., 1x, 5x, 10x, 20x or 100x). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1x, 5x, 10x, 20x or 100x) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### CAR T Cells, ST4/CD3-Directed Bispecific T-cell Engagers, Immunocytokines, Antibody-Drug Conjugates, and Immunotoxins

Another aspect of the present disclosure provides a molecule or complex comprising an antigen-binding site that binds 5T4 as disclosed herein. Exemplary molecules or complexes include but are not limited to chimeric antigen receptors (CARs), T-cell engagers (e.g., ST4/CD3-directed bispecific T-cell engagers), immunocytokines, antibody-drug conjugates, and immunotoxins.

Any antigen-binding site that binds 5T4 as disclosed herein can be used. In certain embodiments, the VH, VL, and/or CDR sequences of the antigen-binding site that binds 5T4 are provided in Table 1. In certain embodiments, the antigen-binding site that binds 5T4 is an scFv. In certain embodiments, the scFv comprises an amino acid sequence at least 90% (e.g., at least 91%, at least 92%, at least 93%, at least 94%, at least 95%, at least 96%, at least 97%, at least 98%, or at least 99%) identical to an amino acid sequence selected from SEQ ID NOs: 13, 14, 18, 19, 23, 25, 27, 29, 31, 34, 36, 38, 40, 60, 61, 95, 96, 97, 98, 99, 100, 101, 102, 103, 104, 105, 106, 107, 120, 121, 123, 124, 126, 128, 133, 134, 135, 136, or 137. In certain embodiments, the scFv comprises an amino acid sequence selected from SEQ ID NOs: 13, 14, 18, 19, 23, 25, 27, 29, 31, 34, 36, 38, 40, 60, 61, 95, 96, 97, 98, 99, 100, 101, 102, 103, 104, 105, 106, 107, 120, 121, 123, 124, 126, 128, 133, 134, 135, 136, or 137.

In certain embodiments, the antigen-binding site that binds 5T4 in the molecule or complex (e.g., CAR, T-cell engager, immunocytokine, antibody-drug conjugate, or immunotoxin) comprises a heavy chain variable domain comprising a CDR1, CDR2, and CDR3 sequences represented by the amino acid sequences of SEQ ID NOs: 3, 4, and 5, respectively; and a light chain variable domain comprising a CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 9; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 13 or SEQ ID NO: 14. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 13.

### Chimeric Antigen Receptors (CARs)

In certain embodiments, the present disclosure provides a 5T4-targeting CAR comprising an antigen-binding site that binds 5T4 as disclosed herein (see, e.g., Table 1). The 5T4-targeting CAR can comprise a Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional cytoplasmic signaling domain derived from a stimulatory molecule (also referred to herein as a “primary signaling domain”).

Accordingly, in certain embodiments, the CAR comprises an extracellular antigen-binding site that binds 5T4 as disclosed herein, a transmembrane domain, and an intracellular signaling domain comprising a primary signaling domain. In certain embodiments, the CAR further comprises one or more functional cytoplasmic signaling domains derived from at least one costimulatory molecule (also referred to as a “costimulatory signaling domain”).

In certain embodiments, the CAR comprises a chimeric fusion protein comprising an antigen-binding site that binds 5T4 (e.g., 5T4-binding scFv) disclosed herein as an extracellular antigen binding domain, a transmembrane domain, and an intracellular signaling domain comprising a primary signaling domain. In certain embodiments, the CAR comprises a chimeric fusion protein comprising an antigen-binding site that binds 5T4 (e.g., 5T4-binding scFv) disclosed herein as an extracellular antigen binding domain, a transmembrane domain, and an intracellular signaling domain comprising a costimulatory signaling domain and a primary signaling domain. In certain embodiments, the CAR comprises a chimeric fusion protein comprising an antigen-binding site that binds 5T4 (e.g., 5T4-binding scFv) disclosed herein as an extracellular antigen binding domain, a transmembrane domain, and an intracellular signaling domain comprising two costimulatory signaling domains and a primary signaling domain. In certain embodiments, the CAR comprises a chimeric fusion protein comprising an antigen-binding site that binds 5T4 (e.g., 5T4-binding scFv) disclosed herein as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 13 or SEQ ID NO: 14. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 13. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:95. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:96.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 41, 42, and 43, respectively; and a light chain variable domain comprising CDR1, CDR2, and CDR3 sequences represented by the amino acid sequences of SEQ ID NOs: 44, 45, and 46, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 15 or 17; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 16. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 18 or SEQ ID NO: 19. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 19. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 120.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:22; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:23.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:24; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:25.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 138; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:27.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:26; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 121.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 108; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 133.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 106, and a VL that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 107.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:28; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:29.

For example, in certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 3, 4, and 5, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:30; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 10. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:31.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:32; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:34.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 122; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 123.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20 and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:36.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:37; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:38.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 125; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 126.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:39; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:40.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 139; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 124.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 53, 54, and 55, respectively; and a light chain variable domain comprising CDR1, CDR2, and CDR3 sequences represented by the amino acid sequences of SEQ ID NOs: 56, 57, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:59. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:60 or 61. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 136 or 137.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 57, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:90; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:97.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 49, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:90; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 92, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:90; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:99.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 35, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 100.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 127; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 128.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 35, respectively; and a light chain variable domain comprising CDR1, CDR2, and CDR3 sequences represented by the amino acid sequences of SEQ ID NOs: 49, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 101.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 35, respectively; and a light chain variable domain comprising CDR1, CDR2, and CDR3 sequences represented by the amino acid sequences of SEQ ID NOs: 92, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:93; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 102.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 50, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20 and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 103.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 49, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20 and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:21. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 104.

In certain embodiments, the extracellular antigen binding domain comprises an antigen-binding site (e.g., an scFv) comprising a heavy chain variable domain comprising CDR1, CDR2, and CDR3 sequences represented by the amino acid sequences of SEQ ID NOs: 47, 4, and 48, respectively; and a light chain variable domain comprising CDR1, CDR2, and CDR3 sequences represented by the amino acid sequences of SEQ ID NOs: 92, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:20 and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:91. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 105.

In certain embodiments, the extracellular antigen binding domain comprises an antigen-binding site (e.g., an scFV) that comprises a heavy chain variable domain comprising CDR1, CDR2, and CDR3 sequences represented by the amino acid sequences from Table 5 and light chain variable domain comprising CDR1, CDR2, and CDR3 sequences represented by the amino acid sequences of, respectively. For example, in certain embodiments, the extracellular antigen binding domain comprises a heavy chain variable domain comprising a CDR1, a CDR2, and a CDR3 sequence selected from the group consisting of: (a) GYTFTSY (SEQ ID NO:53), DSSDSK (SEQ ID NO:54), and GGYLWFAY (SEQ ID NO:55); (b) GYTFGSY (SEQ ID NO:73), DASTEK (SEQ ID NO:74), and GGYLWFQY (SEQ ID NO:75); (c) GYLFTSY (SEQ ID NO:76), SVSDAK (SEQ ID NO:77), and GGYLWFKY (SEQ ID NO:78); (d) GYTFGSY (SEQ ID NO:73), DARSAK (SEQ ID NO:79), and GGYLWFKY(SEQ ID NO:78); (e) GYRFTSY (SEQ ID NO:80), DASSAK (SEQ ID NO:81), and GGYLWFKY (SEQ ID NO:78); (f) GYGFTSY (SEQ ID NO:82), DARTAK (SEQ ID NO:83), and GGYLWYAY (SEQ ID NO:84); (g) GYTFTSY (SEQ ID NO:53), DASDAK (SEQ ID NO:85), and GGYLWYHY (SEQ ID NO:86); (h) GYTFTSY (SEQ ID NO:53), DASDAK (SEQ ID NO:85), and GGYLWYSY (SEQ ID NO:87); (i) GYTFTSY (SEQ ID NO:53), DASDAK (SEQ ID NO:85), and GGYLWYAY (SEQ ID NO:84); (j) GYSFTSY (SEQ ID NO:88), DASDAK (SEQ ID NO:85), and GGYLWFKY (SEQ ID NO:78); (k) GYTFTSY (SEQ ID NO:53), DASDAK (SEQ ID NO:85), and GGYLWFKY (SEQ ID NO:78); and (1) GYGFTSY (SEQ ID NO:82), DARTAK (SEQ ID NO:83), and GGHLWYAY (SEQ ID NO:89). Such extracellular antigen binding domains that bind to 5T4 can be formed by combining any one of these heavy chain variable domains with a light chain variable domain comprising a CDR1, a CDR2, and a CDR3 sequence comprising the amino acid sequences of SEQ ID NOs: 56, 57, and 8, respectively.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR a chain, TCR β chain, TCR ζ chain, CD28, CD3s, CD45, CD4, CD5, CD8, CD9, CD16, CD22, BAFF-R, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R0, IL2Rγ, IL7Rα, ITGA1, VLA1, CD49a, ITGA4, IA4, CD49D, ITGA6, VLA-6, CD49f, ITGAD, CD11d, ITGAE, CD 103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD 150, IPO-3), BLAME (SLAMF8), SELPLG (CD 162), LTBR, PAG/Cbp, NKG2D, and NKG2C.

The extracellular 5T4-binding domain (e.g., 5T4-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker (SEQ ID NO: 111), a KIR2DS2 hinge, and a CD8a hinge.

The intracellular signaling domain of the CAR of the present disclosure is responsible for activation of at least one of the specialized functions of the immune cell (e.g., cytolytic activity or helper activity, including the secretion of cytokines, of a T cell) in which the CAR has been placed. Thus, as used herein, the term “intracellular signaling domain” refers to the portion of a protein which transduces an effector function signal and directs the cell to perform a specialized function. Although usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional cytoplasmic signaling domain derived from a stimulatory molecule) and one or more costimulatory signaling domains (i.e., functional cytoplasmic signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present disclosure include those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, the primary signaling domain in any one or more CARs of the present disclosure comprises a cytoplasmic signaling sequence derived from CD3-zeta.

In some embodiments, the primary signaling domain is a functional cytoplasmic signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional cytoplasmic signaling domain of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and/or DAP12. In a particular embodiment, the primary signaling domain is a functional cytoplasmic signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD I I a/CD 18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 11d, ITGAE, CD 103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional cytoplasmic signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD1 la/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR of the present disclosure may be linked to each other in a random or specified order. Optionally, a short oligo- or polypeptide linker, for example, between 2 and 10 amino acids in length may form the linkage.

Another aspect of the present disclosure provides a nucleic acid encoding a 5T4-targeting CAR disclosed herein. The nucleic acid is useful for expressing the CAR in an effector cell (e.g., T cell) by introducing the nucleic acid to the cell.

Modifications may be made in the sequence to create an equivalent or improved variant of the present disclosure, for example, by changing one or more of the codons according to the codon degeneracy table. A DNA codon degeneracy table is provided in Table 4.

TABLE 4

Amino Acid Codons

| | | | |
|---|---|---|---|
| Amino Acids | One letter code | Three letter code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG CGU |
| Serine | S | Ser | AGC AGU UCA UCC UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present disclosure provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular 5T4-binding domain (e.g., 5T4-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

Another aspect of the present disclosure provides an immune effector cell expressing the 5T4-targeting CAR. Also provided is an immune effector cell comprising the nucleic acid encoding the 5T4-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present disclosure provides a pluripotent or multipotent cell (e.g., a hematopoietic stem cell) expressing the 5T4-targeting CAR (e.g., expressing the CAR on the plasma membrane) or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present disclosure is a cell obtained from in vitro expansion.

Further exemplary methods and applications of the 5T4-targeting CAR (e.g., regulatable CAR), nucleic acid encoding the CAR, and effector cells expressing the CAR or comprising the nucleic acid are provided in U.S. Pat. Nos. 7,446,190 and 9,181,527, U.S. Pat. Application Publication Nos. 2016/0340406 and 2017/0049819, and International Pat. Application Publication No. WO2018/140725.

### 5T4/CD3-Directed Bispecific T-Cell Engagers

In certain embodiments, the present disclosure provides a 5T4/CD3-directed bispecific T-cell engager comprising an antigen-binding site that binds 5T4 disclosed herein. In certain embodiments, the 5T4/CD3-directed bispecific T-cell engager comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NO:13 or 14. In certain embodiments, the 5T4/CD3-directed bispecific T-cell engager comprises an amino acid sequence of SEQ ID NO:95 or SEQ ID NO:96. In certain embodiments, the cytokine is connected to the Fc domain directly or via a linker.

In certain embodiments, the 5T4/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present disclosure provides a nucleic acid encoding at least one polypeptide of the 5T4/CD3-directed bispecific T-cell engager, wherein the polypeptide comprises an antigen-binding site that binds 5T4. In certain embodiments, the nucleic acid further comprises a nucleotide sequence encoding a signal peptide that, when expressed, is at the N-terminus of one or more of the polypeptides of the 5T4/CD3-directed bispecific T-cell engager. Also provided is a vector (e.g., a viral vector) comprising the nucleic acid, a producer cell comprising the nucleic acid or vector, and a producer cell expressing the 5T4/CD3-directed bispecific T-cell engager.

### Immunocytokines

In certain embodiments, the present disclosure provides an immunocytokine comprising an antigen-binding site that binds 5T4 disclosed herein and a cytokine. Any cytokine (e.g., pro-inflammatory cytokines) known in the art can be used, including but not limited to IL-2, IL-4, IL-10, IL-12, IL-15, TNF, IFNα, IFNγ, and GM-CSF. Other exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of each polypeptide. The immunocytokine can further comprise an Fc domain connected to the antigen-binding site that binds 5T4. In certain embodiments, the immunocytokine comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 13 or 14. In certain embodiments, the immunocytokine comprises an amino acid sequence of SEQ ID NO:95 or SEQ ID NO:96. In certain embodiments, the cytokine is connected to the Fc domain directly or via a linker.

Another aspect of the present disclosure provides a nucleic acid encoding at least one polypeptide of the immunocytokine, wherein the polypeptide comprises an antigen-binding site that binds 5T4. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present disclosure provides an antibody-drug conjugate comprising an antigen-binding site that binds 5T4 disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds 5T4. In certain embodiments, the antibody-drug conjugate comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 13 or 14. In certain embodiments, the antibody-drug conjugate comprises an amino acid sequence of SEQ ID NO:95 or SEQ ID NO:96. In certain embodiments, the cytotoxic drug moiety is connected to the Fc domain directly or via a linker.

### Immunotoxins

In certain embodiments, the present disclosure provides an immunotoxin comprising an antigen-binding site that binds 5T4 disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds 5T4. In certain embodiments, the immunotoxin comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from SEQ ID NOs: 13 or 14. In certain embodiments, the immunotoxin comprises an amino acid sequence of SEQ ID NO:95 or SEQ ID NO:96. In certain embodiments, the cytotoxic peptide moiety is connected to the Fc domain directly or via a linker.

Another aspect of the present disclosure provides a nucleic acid encoding at least one polypeptide of the immunotoxin, wherein the polypeptide comprises an antigen-binding site that binds 5T4. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

### II. Therapeutic Compositions and Their Use

The present disclosure provides methods for treating cancer using a protein, conjugate, or cells comprising an antigen-binding site disclosed herein and/or a pharmaceutical composition described herein. Such methods include administering to a subject in need thereof an effective amount of any one of the antigen-binding sites or proteins described herein, including administering to a subject in need thereof the antigen-binding site or protein in the form of an effective amount of the antigen-binding site or protein, or a pharmaceutical composition, formulation, or dosage thereof described herein. The antigen-binding sites or proteins can be administered to a subject using any route well known in the art for administration of an antibody or antibody fragment. The methods may be used to treat a variety of cancers which express 5T4 by administering to a patient in need thereof a therapeutically effective amount of a protein, conjugate, or cells comprising an antigen-binding site disclosed herein. The methods of the present application can improve a variety of clinical endpoints. For example, in some embodiments, the method increases overall survival in the subject relative to individuals not receiving treatment. In some embodiments, the method increases progression free survival in the subject relative to individuals not receiving treatment. In some embodiments, the method increases overall survival and progression free survival in the subject relative to individuals not receiving treatment.

The therapeutic method of the present application can be characterized according to the cancer to be treated. The cancer to be treated can be characterized according to the presence of a particular antigen expressed on the surface of the cancer cell, e.g., 5T4. Accordingly, in some embodiments, the 5T4 is expressed by cancer cells. In some embodiments, the 5T4 is expressed by cancer-associated fibroblasts. In some embodiments, the 5T4 is expressed at high levels relative to normal cells. In some embodiments, the 5T4 is expressed at low levels relative to normal cells.

Cancers characterized by the expression of 5T4, include, for example and without limitation, colorectal cancer, ovarian cancer, cervical cancer, lung (e.g., non-small cell lung cancer), renal cancer, bladder cancer, prostate cancer, breast cancer (e.g., hormone receptor positive (HR+) breast cancer), uterine cancer, endometrial cancer, squamous cell carcinoma, head and neck squamous cell carcinoma, uterine cancer, pancreatic cancer, mesothelioma, esophageal cancer, and gastric cancer. See, e.g., Stern, et al., Cancer Immunol Immunother (2017) 66:415-426.

It is contemplated that the protein, conjugate, cells, and/or pharmaceutical compositions described in the present disclosure can be used to treat a variety of cancers, not limited to cancers in which the cancer cells or the cells in the cancer microenvironment express 5T4. It is also contemplated that the subject treated with the protein, conjugate, cells, and/or pharmaceutical compositions described in the present disclosure has previously received treatment, including chemotherapy for cancer. As such, in some embodiments, the subject treated by the protein, conjugate, cells, and/or pharmaceutical compositions described in the present disclosure is refractory to chemotherapy.

In certain embodiments, the cancer is a solid tumor. In certain embodiments, the cancer is a metastatic cancer. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing’s sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin’s disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi’s sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma, nodular melanoma, non-epithelial skin cancer, non-Hodgkin’s lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain embodiments, the cancer is selected from the group consisting of colorectal cancer, ovarian cancer, cervical cancer, lung (e.g., non-small cell lung cancer), renal cancer, bladder cancer, prostate cancer, breast cancer (e.g., hormone receptor positive (HR+) breast cancer), uterine cancer, endometrial cancer, squamous cell carcinoma, head and neck squamous cell carcinoma, uterine cancer, pancreatic cancer, mesothelioma, esophageal cancer, and gastric cancer. In certain embodiments, the cancer is selected from the group consisting of breast cancer, cervical cancer, lung (e.g., non-small cell lung cancer), renal cancer, bladder cancer, head and neck squamous cell carcinoma, pancreatic cancer, and gastric cancer.

The present disclosure also provides methods of enhancing tumor cell death. Such methods include exposing the tumor cell to an effective amount of any one of the antigen-binding sites or proteins described herein, an antibody-drug conjugate described herein, an immunocytokine described herein, a bispecific T-cell engager described herein, a CAR described herein, or an immune effector cell described herein, including administering to a subject in need thereof an effective amount of any one of the antigen-binding sites or proteins described herein, an antibody-drug conjugate described herein, an immunocytokine described herein, a bispecific T-cell engager described herein, a CAR described herein, or an immune effector cell described herein.

### III. Combination Therapies

Another aspect of the present application provides for combination therapy. A protein described herein can be used in combination with additional therapeutic agents to treat cancer.

Exemplary therapeutic agents that may be used as part of a combination therapy in treating cancer include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton’s Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-2, IL-12, IL-15, GM-CSF, G-CSF and variants thereof.

In some embodiments, the 5T4-targeting antigen-binding site or antigen-binding domain, as described herein, is co-administered with one or more therapeutic agents selected from a PI3K inhibitor, a FLT3R agonist, a PD-1 antagonist, a PD-L1 antagonist, a CD47 inhibitor, a Trop-2 inhibitor, an MCL1 inhibitor, a CCR8 binding agent, an HPK1 antagonist, a DGKα inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments, the 5T4-targeting antigen-binding site or antigen-binding domain, as described herein, is co-administered with one or more therapeutic agents selected from a PI3Kδ inhibitor (e.g., idealisib), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a CD47 inhibitor (e.g., magrolimab), a Trop-2 inhibitor (e.g., sacituzumab govitecan (TRODELVY™)), a small molecule MCL1 inhibitor (e.g., GS-9716), a small molecule HPK1 inhibitor (e.g., GS-6451), a HPK1 degrader (PROTAC; e.g., ARV-766), a small molecule DGKα inhibitor, a small molecule CD73 inhibitor (e.g., quemliclustat (AB680)), an anti-CD73 antibody (e.g., oleclumab), a dual A2a/A2b adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223), an IL-2 receptor agonist (e.g., GS-4528), and a CAR-T cell therapy (e.g., axicabtagene ciloleucel, brexucabtagene autoleucel, tisagenlecleucel).

In some embodiments, the 5T4-targeting antigen-binding site or antigen-binding domain, as described herein, is co-administered with one or more therapeutic agents selected from magrolimab, sacituzumab govitecan (TRODELVY™), GS-4528, idealisib, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel, and brexucabtagene autoleucel.

In various embodiments, the 5T4-targeting antigen-binding site or antigen-binding domain, as described herein, is further combined with an agent that inhibits binding between CD47 and SIRPα. In various embodiments, the agent that inhibits binding between CD47 and SIRPα is an antibody or antigen-binding fragment thereof that binds to CD47 (a.k.a., IAP, MER6, OA3; NCBI Gene ID: 961; UniProt Q08722). In various embodiments, an antibody that binds to CD47 has an Fc having effector function. In various embodiments, an antibody that binds to CD47 is an IgG4 or an IgG1. Examples of anti-CD47 antibodies of use include without limitation: magrolimab, lemzoparlimab, letaplimab, ligufalimab (AK117), AO-176, IBI-322, ZL-1201, IMC-002, SRF-231, CC-90002 (a.k.a., INBRX-103), NI-1701 (a.k.a., TG-1801), STI-6643 (Vx-1004), CNTO-7108, RCT-1938, RRx-001, DSP-107, VT-1021, and SGN-CD47M.

In various embodiments, the agent that inhibits binding between CD47 and SIRPα CD47 is an antibody or antigen-binding fragment thereof that binds to signal regulatory protein alpha (SIRPα) (NCBI Gene ID: 140885; UniProt P78324). Illustrative antibodies that bind to SIRPα include without limitation: anzurstobart (a.k.a., CC-95251), GS-0189 (FSI-189), ES-004, BI765063, and ADU1805.

In various embodiments, the agent that inhibits binding between CD47 and SIRPα CD47 is a SIRPα-Fc fusion protein or a “high affinity SIRPα reagent”, which includes SIRPα-derived polypeptides and analogs thereof. Illustrative SIRPα-Fc fusion proteins of use include ALX-148 (a.k.a., evorpacept, described in WO2013109752), TTI-621 or TTI-622 (described in WO2014094122), SIRPa-F8, JY002-M2G1(N297A), JMT601 (CPO107), SS002M91, SIRPalpha-lgG4-Fc-Fc, and hCD172a(SIRPa)-Fc-LIGHT.

In various embodiments, the 5T4-targeting antigen-binding site or antigen-binding domain, as described herein, is further combined with an agonist of fms related receptor tyrosine kinase 3 (FLT3); FLK2; STK1; CD135; FLK-2; NCBI Gene ID: 2322). Examples of FLT3 agonists include, but are not limited to, CDX-301, and GS-3583. GS-3583 is described, e.g., in WO 2020/263830, hereby incorporated herein by reference in its entirety for all purposes.

In various embodiments, the 5T4-targeting antigen-binding site or antigen-binding domain, as described herein, is further combined with an anti-CD19 agent or antibody. Examples of anti-CD19 agents or antibodies that can be co-administered include without limitation: blinatumomab, tafasitamab, XmAb5574 (Xencor), AFM-11, inebilizumab, loncastuximab, MEDI 551 (Cellective Therapeutics); and MDX-1342 (Medarex).

In various embodiments, the 5T4-targeting antigen-binding site or antigen-binding domain, as described herein, is further combined with an anti-CD20 agent or antibody. Examples of anti-CD20 agents or antibodies that can be co-administered include without limitation: IGN-002, PF-05280586; Rituximab (Rituxan/Biogen Idec), Ofatumumab (Arzerra/Genmab), Obinutuzumab (Gazyva/Roche Glycart Biotech), Alemtuzumab, Veltuzumab, Veltuzumab, Ocrelizumab (Ocrevus/Biogen Idec; Genentech), Ocaratuzumab and Ublituximab, and LFB-R603 (LFB Biotech.; rEVO Biologics).

In various embodiments, the 5T4-targeting antigen-binding site or antigen-binding domain, as described herein, is further combined with an anti-CD22 agent or antibody. Examples of anti-CD22 agents or antibodies that can be co-administered include without limitation: Epratuzumab, AMG-412, and IMMU-103 (Immunomedics).

In various embodiments, the 5T4-targeting antigen-binding site or antigen-binding domain, as described herein, is further combined with an anti-CD30 agent or antibody. Examples of anti-CD30 agents or antibodies that can be co-administered include without limitation: Brentuximab vedotin (Seattle Genetics).

In various embodiments, the 5T4-targeting antigen-binding site or antigen-binding domain, as described herein, is further combined with an anti-CD33 agent or antibody. Examples of anti-CD33 agents or antibodies that can be co-administered include without limitation: gemtuzumab, lintuzumab, vadastuximab, CIK-CAR.CD33; CD33CART, AMG-330 (CD33/CD3), AMG-673 (CD33/CD3),GEM-333 (CD3/CD33), and IMGN-779.

In various embodiments, the 5T4-targeting antigen-binding site or antigen-binding domain, as described herein, is further combined with an anti-CD37 agent or antibody. Examples of anti- CD37 agents or antibodies that can be co-administered include without limitation: BI836826 (Boehringer Ingelheim), Otlertuzumab, and TRU-016 (Trubion Pharmaceuticals).

In various embodiments, the 5T4-targeting antigen-binding site or antigen-binding domain, as described herein, is further combined with an anti-CD38 agent or antibody. Examples of anti-CD38 agents or antibodies that can be co-administered include without limitation: CD38, such as T-007, UCART-38; Darzalex (Genmab), Daratumumab, JNJ-54767414 (Darzalex/Genmab), Isatuximab, SAR650984 (ImmunoGen), MOR202, MOR03087 (MorphoSys), TAK-079; and anti-CD38-attenukine, such as TAK573.

In various embodiments, the 5T4-targeting antigen-binding site or antigen-binding domain, as described herein, is further combined with an anti-CD52 agent or antibody. Examples of anti-CD52 agents or antibodies that can be co-administered include without limitation: anti-CD52 antibodies, such as Alemtuzumab (Campath/University of Cambridge).

In various embodiments, the 5T4-targeting antigen-binding site or antigen-binding domain, as described herein, is further combined with an anti-CD98 (4F2, FRP-1) agent or antibody. Examples of anti-CD98 agents or antibodies that can be co-administered include without limitation: IGN523 (Igenica).

In various embodiments, the 5T4-targeting antigen-binding site or antigen-binding domain, as described herein, is further combined with an anti-CD157 (BST-1) agent or antibody. Examples of anti-CD157 agents or antibodies that can be co-administered include without limitation: OBT357, and MEN1112 (Menarini; Oxford BioTherapeutics).

In various embodiments, the 5T4-targeting antigen-binding site or antigen-binding domain, as described herein, is further combined with an anti- DKK-1 agent or antibody. Examples of anti-DKK-1 agents or antibodies that can be co-administered include without limitation: BHQ880 (MorphoSys; Novartis), and DKN-01, LY-2812176 (Eli Lilly).

In various embodiments, the 5T4-targeting antigen-binding site or antigen-binding domain, as described herein, is further combined with an anti-GRP78 (BiP) agent or antibody. Examples of anti-GRP78 agents or antibodies that can be co-administered include without limitation: PAT-SM6 (OncoMab GmbH).

In various embodiments, the 5T4-targeting antigen-binding site or antigen-binding domain, as described herein, is further combined with an anti-NOTCH1 agent or antibody. Examples of anti-NOTCH1 agents or antibodies that can be co-administered include without limitation: Brontictuzumab, and OMP-52M51 (OncoMed Pharmaceuticals).

In various embodiments, the 5T4-targeting antigen-binding site or antigen-binding domain, as described herein, is further combined with an anti-ROR1 agent or antibody. Examples of anti- ROR1 agents or antibodies that can be co-administered include without limitation: Mapatumumab, TRM1, and HGS-1012 (Cambridge Antibody Technology).

In various embodiments, the 5T4-targeting antigen-binding site or antigen-binding domain, as described herein, is further combined with an anti-SLAMF7 (CS1, CD319) agent or antibody. Examples of anti-SLAMF7 agents or antibodies that can be co-administered include without limitation: Elotuzumab, HuLuc63, BMS-901608 (Empliciti/PDL BioPharma), and Mogamulizumab (KW-0761).

In various embodiments, the 5T4-targeting antigen-binding site or antigen-binding domain, as described herein, is further combined with an anti-TNFRSF10A (DR4; APO2; CD261; TRAILR1; TRAILR-1) agent or antibody. Examples of anti- TNFRSF10A agents or antibodies that can be co-administered include without limitation: Mapatumumab, TRM1, and HGS-1012 (Cambridge Antibody Technology).

In various embodiments, the 5T4-targeting antigen-binding site or antigen-binding domain, as described herein, is further combined with an anti-Transferrin Receptor (TFRC; CD71) agent or antibody. Examples of anti-Transferrin Receptor agents or antibodies that can be co-administered include without limitation: E2.3/A27.15 (University of Arizona).

In various embodiments, the 5T4-targeting antigen-binding site or antigen-binding domain, as described herein, is further combined with an anti-EPHA3 agent or antibody. Examples of anti-EPHA3 agents or antibodies that can be co-administered include without limitation: Ifabotuzumab, and KB004 (Ludwig Institute for Cancer Research).

In various embodiments, the 5T4-targeting antigen-binding site or antigen-binding domain, as described herein, is further combined with an anti-CCR4 agent or antibody. Examples of anti- CCR4 agents or antibodies that can be co-administered include without limitation: Mogamulizumab, and KW-0761 (Poteligeo/Kyowa Hakko Kirin Co.).

In various embodiments, the 5T4-targeting antigen-binding site or antigen-binding domain, as described herein, is further combined with an anti-CXCR4 agent or antibody. Examples of anti-CXCR4 agents or antibodies that can be co-administered include without limitation: Ulocuplumab, BMS-936564, MDX-1338 (Medarex), and PF-06747143 (Pfizer).

In various embodiments, the 5T4-targeting antigen-binding site or antigen-binding domain, as described herein, is further combined with an anti-BAFF agent or antibody. Examples of anti-BAFF agents or antibodies that can be co-administered include without limitation: Tabalumab, and LY2127399 (Eli Lilly).

In various embodiments, the 5T4-targeting antigen-binding site or antigen-binding domain, as described herein, is further combined with an anti-BAFF Receptor (BAFF-R) agent or antibody. Examples of anti-BAFF-R agents or antibodies that can be co-administered include without limitation: VAY736 (MorphoSys; Novartis).

In various embodiments, the 5T4-targeting antigen-binding site or antigen-binding domain, as described herein, is further combined with an anti-RANKL agent or antibody. Examples of anti-RANKL agents or antibodies that can be co-administered include without limitation: Denosumab, and AMG-162 (Prolia; Ranmark; Xgeva/Amgen).

In various embodiments, the 5T4-targeting antigen-binding site or antigen-binding domain, as described herein, is further combined with an anti-IL-6 agent or antibody. Examples of anti-IL-6 agents or antibodies that can be co-administered include without limitation: Siltuximab, and CNTO-328 (Sylvant/Centocor).

In various embodiments, the 5T4-targeting antigen-binding site or antigen-binding domain, as described herein, is further combined with an anti-IL-6 Receptor (IL-6R) agent or antibody. Examples of anti-IL-6R agents or antibodies that can be co-administered include without limitation: Tocilizumab, R-1569 (Actemra/Chugai Pharmaceutical; Osaka University), and AS-101 (CB-06-02, IVX-Q-101).

In various embodiments, the 5T4-targeting antigen-binding site or antigen-binding domain, as described herein, is further combined with an anti-IL3RA (CD123) agent or antibody. Examples of anti- IL3RA (CD123) agents or antibodies that can be co-administered include without limitation: tagraxofusp, talacotuzumab (JNJ-56022473; CSL362 (CSL)), pivekimab sunirine (IMGN632), MB-102 (Mustang Bio), CSL360 (CSL); vibecotamab (XmAb 14045; Xencor); KHK2823 (Kyowa Hakko Kirin Co.); MGD-024 (CD123/CD3; Macrogenics), APVO436 (CD123/CD3); flotetuzumab (CD123/CD3); JNJ-63709178 (CD123/CD3); and XmAb-14045 (CD123/CD3) (Xencor).

In various embodiments, the 5T4-targeting antigen-binding site or antigen-binding domain, as described herein, is further combined with an anti-IL2RA (CD25) agent or antibody. Examples of anti-IL2RA agents or antibodies that can be co-administered include without limitation: Basiliximab, SDZ-CHI-621 (Simulect/Novartis), and Daclizumab.

In various embodiments, the 5T4-targeting antigen-binding site or antigen-binding domain, as described herein, is further combined with an anti-IGF-1R (CD221) agent or antibody. Examples of anti-IGF-1R agents or antibodies that can be co-administered include without limitation: Ganitumab, AMG-479 (Amgen); Ganitumab, AMG-479 (Amgen), Dalotuzumab, MK-0646 (Pierre Fabre), and AVE1642 (ImmunoGen).

In various embodiments, the 5T4-targeting antigen-binding site or antigen-binding domain, as described herein, is further combined with an anti-GM-CSF (CSF2) agent or antibody. Examples of anti- GM-CSF agents or antibodies that can be co-administered include without limitation: Lenzilumab (a.k.a., KB003; KaloBios Pharmaceuticals).

In various embodiments, the 5T4-targeting antigen-binding site or antigen-binding domain, as described herein, is further combined with an anti-HGF agent or antibody. Examples of anti-HGF agents or antibodies that can be co-administered include without limitation: Ficlatuzumab, AV-299 (AVEO Pharmaceuticals).

In various embodiments, the 5T4-targeting antigen-binding site or antigen-binding domain, as described herein, is further combined with an anti-CD44 agent or antibody. Examples of anti- CD44 agents or antibodies that can be co-administered include without limitation: RG7356, RO5429083 (Chugai Biopharmaceuticals; Roche).

In various embodiments, the 5T4-targeting antigen-binding site or antigen-binding domain, as described herein, is further combined with an anti-VLA-4 (CD49d) agent or antibody. Examples of anti-VLA-4 agents or antibodies that can be co-administered include without limitation: Natalizumab, and BG-0002-E (Tysabri/Elan Corporation).

In various embodiments, the 5T4-targeting antigen-binding site or antigen-binding domain, as described herein, is further combined with an anti-ICAM-1 (CD54) agent or antibody. Examples of anti- ICAM-1 agents or antibodies that can be co-administered include without limitation: BI-505 (BioInvent International).

In various embodiments, the 5T4-targeting antigen-binding site or antigen-binding domain, as described herein, is further combined with an anti-VEGF-A agent or antibody. Examples of anti-VEGF-A agents or antibodies that can be co-administered include without limitation: Bevacizumab (Avastin/Genentech; Hackensack University Medical Center).

In various embodiments, the 5T4-targeting antigen-binding site or antigen-binding domain, as described herein, is further combined with an anti-Endosialin (CD248, TEM1) agent or antibody. Examples of anti-Endosialin agents or antibodies that can be co-administered include without limitation: Ontecizumab, and MORAB-004 (Ludwig Institute for Cancer Research; Morphotek).

In various embodiments, the 5T4-targeting antigen-binding site or antigen-binding domain, as described herein, is further combined with an anti-CD79 agent or antibody. Examples of anti-CD79 agents or antibodies that can be co-administered include without limitation: polatuzumab, DCDS4501A, and RG7596 (Genentech).

In various embodiments, the 5T4-targeting antigen-binding site or antigen-binding domain, as described herein, is further combined with an anti- Isocitrate dehydrogenase (IDH) agent or antibody. Examples of anti-IDH agents or antibodies that can be co-administered include without limitation: IDH1 inhibitor ivosidenib (Tibsovo; Agios) and the IDH2 inhibitor enasidenib (Idhifa; Celgene/Agios).

In various embodiments, the 5T4-targeting antigen-binding site or antigen-binding domain, as described herein, is further combined with an antibody that targets tumor associated calcium signal transducer 2 (TACSTD2) (NCBI Gene ID: 4070; EGP-1, EGP1, GA733-1, GA7331, GP50, M1S1, TROP2), such as sacituzumab, e.g., sacituzumab govitecan (TRODELVY™).

In various embodiments, the 5T4-targeting antigen-binding site or antigen-binding domain, as described herein, is further combined with an anti-major histocompatibility complex, class I, G (HLA-G; NCBI Gene ID: 3135) antibody, such as TTX-080.

In various embodiments, the 5T4-targeting antigen-binding site or antigen-binding domain, as described herein, is further combined with an anti-leukocyte immunoglobulin like receptor B2 (LILRB2, a.k.a., CD85D, ILT4; NCBI Gene ID: 10288) antibody, such as JTX-8064 or MK-4830.

### TNF Receptor Superfamily (TNFRSF) Member Agonists or Activators

In various embodiments, the 5T4-targeting antigen-binding site or antigen-binding domain, as described herein, is further combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628, each of which is hereby incorporated by reference in its entirety.

Examples anti-TNF receptor superfamily member 10b (TNFRSF10B, DR5, TRAILR2) antibodies that can be co-administered include without limitation: DS-8273, CTB-006, INBRX-109, and GEN-1029.

Examples of anti-TNFRSF5 (CD40) antibodies that can be co-administered include without limitation: selicrelumab (RO7009789), mitazalimab (a.k.a., vanalimab, ADC-1013, JNJ-64457107), RG7876, SEA-CD40, APX-005M and ABBV-428, ABBV-927, and JNJ-64457107.

Examples of anti-TNFRSF7 (CD27) that can be co-administered include without limitation: varlilumab (CDX-1127).

Examples of anti-TNFRSF9 (4-1BB, CD137) antibodies that can be co-administered include without limitation: urelumab, utomilumab (PF-05082566), AGEN2373, ADG-106, BT-7480, and QL1806.

Examples of anti-TNFRSF17 (BCMA) that can be co-administered include without limitation: GSK-2857916.

Examples of anti-TNFRSF18 (GITR) antibodies that can be co-administered include without limitation: MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628, each of which is hereby incorporated by reference in its entirety.

Example anti-TRAILR1, anti-TRAILR2, anti-TRAILR3, anti-TRAILR4 antibodies that can be co-administered include without limitation: ABBV-621.

Examples of bi-specific antibodies targeting TNFRSF family members that can be co-administered include without limitation: PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20), and AMG-424 (CD38.CD3).

Examples of inhibitors of PVR related immunoglobulin domain containing (PVRIG, CD112R) that can be co-administered include without limitation: COM-701.

Examples of inhibitors of T cell immunoreceptor with Ig and ITIM domains (TIGIT; NCBI Gene ID: 201633) that can be co-administered include without limitation: BMS-986207, RG-6058, AGEN-1307, and COM-902, etigilimab, tiragolumab (a.k.a., MTIG-7192A; RG-6058; RO 7092284), AGEN1777, IBI-939, AB154, MG1131, and EOS884448 (EOS-448).

Examples of inhibitors of hepatitis A virus cellular receptor 2 (HAVCR2, TIMD3, TIM-3) that can be co-administered include without limitation: cobolimab (TSR-022), LY-3321367, sabatolimab (MBG-453), INCAGN-2390, RO-7121661 (PD-1/TIM-3), LY-3415244 (TIM-3/PDL1), and RG7769 (PD-1/TIM-3).

Examples of inhibitors of lymphocyte activating 3 (LAG-3, CD223) that can be co-administered include without limitation: relatlimab (ONO-4482), LAG-525, MK-4280, REGN-3767, INCAGN2385, TSR-033, MGD-013 (PD-1/LAG-3), and FS-118 (LAG-3/PD-L1).

Examples of anti-V-set immunoregulatory receptor (VSIR, B7H5, VISTA) antibodies that can be co-administered include without limitation: HMBD-002, and CA-170 (PD-L1/VISTA).

Examples of anti-CD70 antibodies that can be co-administered include without limitation: AMG-172.

Examples of anti-ICOS antibodies that can be co-administered include without limitation: JTX-2011, and GSK3359609.

Examples of ICOS agonists that can be co-administered include without limitation: ICOS-L.COMP (Gariepy, et al. 106th Annu Meet Am Assoc Immunologists (AAI) (May 9-13, San Diego) 2019, Abst 71.5).

### Immune Checkpoint Inhibitors

In various embodiments, the 5T4-targeting antigen-binding site or antigen-binding domain, as described herein, is further combined with one or more immune checkpoint inhibitors. In some embodiments, the one or more immune checkpoint inhibitors is a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation: ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD 1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors/antibodies of PD-L1 (CD274) or PD-1 (PDCD1) that can be co-administered include without limitation: zimberelimab, pembrolizumab (KEYTRUDA®, MK-3477), nivolumab (OPDIVO®, BMS-936558, MDX-1106), cemiplimab, pidilizumab, spartalizumab (PDR-001), atezolizumab (RG 7446; TECENTRIQ, MPDL3280A), durvalumab (MEDI-4736), avelumab (MSB0010718C), tislelizumab (BGB-A317), toripalimab (JS-001), genolimzumab (CBT-501), camrelizumab (SHR-1210), dostarlimab (TSR-042), sintilimab (IBI-308), tislelizumab (BGB-A317), cemiplimab (REGN-2810), lambrolizumab (CAS Reg. No. 1374853-91-4), AMG-404, AMP-224, MEDI0680 (AMP-514), BMS-936559, CK-301, PF-06801591, GEN-1046 (PD-L¼-1BB), GLS-010 (WBP-3055), AK-103 (HX-008), AK-105, CS-1003, HLX-10, MGA-012, BI-754091, AGEN-2034, JNJ-63723283, LZM-009, BCD-100, LY-3300054, SHR-1201, Sym-021, ABBV-181, PD1-PIK, BAT-1306, CX-072, CBT-502, MSB-2311, JTX-4014, BGB-A333, SHR-1316, CS-1001 (WBP-3155, KN-035, HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), SCH-900475, PF-06801591, AGEN-2034, AK-105, PD1-PIK, BAT-1306, BMS-936559, CK-301, MEDI-0680, PDR001 + Tafinlar ® + Mekinist ®, and those described, e.g., in Intl. Patent Publ. Nos. WO2018195321, WO2020014643, WO2019160882, and WO2018195321.

In various embodiments, the 5T4-targeting antigen-binding site or antigen-binding domain, as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, and WO2017147410. Toll-Like Receptor (TLR) Agonists

In various embodiments, the 5T4-targeting antigen-binding site or antigen-binding domain, or an anti-SIRPα agent as described herein, is combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation: DS-0509, GS-9620, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation: E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation: AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10, and PUL-042. Examples of TLR3 agonist include rintatolimod, poly-ICLC, RIBOXXON®, Apoxxim, RIBOXXIM®, IPH-33, MCT-465, MCT-475, and ND-1.1.

Examples of TLR8 inhibitors include, but are not limited to, E-6887, IMO-8400, IMO-9200, and VTX-763.

Examples of TLR8 agonists include, but are not limited to, MCT-465, motolimod, GS-9688, and VTX-1463.

Examples of TLR9 agonists include but are not limited to, AST-008, IMO 2055, IMO-2125, lefitolimod, litenimod, MGN-1601, and PUL-042.

Examples of TLR7/TLR8 agonists include without limitation: NKTR-262, IMO-4200, MEDI-9197 (telratolimod), and resiquimod.

Examples of TLR agonists include without limitation: lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, and telratolimod.

In some embodiments, the therapeutic agent is a stimulator of interferon genes (STING) In some embodiments, the STING receptor agonist or activator is selected from ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### Hematopoietic Progenitor Kinase 1 (HPK1) Inhibitors

In various embodiments, the 5T4-targeting antigen-binding site or antigen-binding domain, as described herein, is further combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152, WO2020092528, WO2020092621, and WO-2016090300.

### Apoptosis Signal-Regulating Kinase (ASK) Inhibitors

In various embodiments, the 5T4-targeting antigen-binding site or antigen-binding domain, as described herein, is further combined with an inhibitor of an ASK inhibitor, e.g., mitogen-activated protein kinase kinase kinase 5 (MAP3K5; ASK1, MAPKKK5, MEKKS; NCBI Gene ID: 4217). Examples of ASK1 inhibitors include without limitation, those described in WO 2011/008709 (Gilead Sciences) and WO 2013/112741 (Gilead Sciences). Bruton Tyrosine Kinase (BTK) Inhibitors

In various embodiments, the 5T4-targeting antigen-binding site or antigen-binding domain, as described herein, is further combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, Calquence + AZD6738, and Calquence + danvatirsen.

### Cyclin-Dependent Kinase (CDK) Inhibitors

In various embodiments, the 5T4-targeting antigen-binding site or antigen-binding domain, as described herein, is further combined with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include without limitation: abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02. Discoidin Domain Receptor (DDR) Inhibitors.

In various embodiments, the 5T4-targeting antigen-binding site or antigen-binding domain, as described herein, is further combined with an inhibitor of discoidin domain receptor tyrosine kinase 1 (DDR1, CAK, CD 167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Histone Deacetylase (HDAC) Inhibitors

In various embodiments, the 5T4-targeting antigen-binding site or antigen-binding domain, as described herein, is further combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation: abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat, romidepsin, and tucidinostat.

### Indoleamine-Pyrrole-2,3-Dioxygenase (IDO1) Inhibitors

In various embodiments, the 5T4-targeting antigen-binding site or antigen-binding domain, as described herein, is further combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Janus Kinase (JAK) Inhibitors

In various embodiments, the 5T4-targeting antigen-binding site or antigen-binding domain, as described herein, is further combined with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3 _HUMAN, JAKL, L-JAK, LJAK; NCBI Gene ID: 3718). Examples of JAK inhibitors include without limitation: AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

### Matrix Metalloprotease (MMP) Inhibitors

In various embodiments, the 5T4-targeting antigen-binding site or antigen-binding domain, as described herein, is further combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066), and/or MMP28 (NCBI Gene ID: 79148). Examples of MMP9 inhibitors include without limitation, marimastat (BB-2516), cipemastat (Ro 32-3555), GS-5745 (andecaliximab), and those described in WO 2012/027721 (Gilead Biologics).

### RAS and RAS Pathway Inhibitors

In various embodiments, the 5T4-targeting antigen-binding site or antigen-binding domain, as described herein, is further combined with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASH1; RASK2; Ki-Ras; p21ras; C-H-RAS; c-K-ras; H-RASIDX; c-Ki-ras; C-BAS/HAS; C-HA-RAS1; and NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT, and mTOR.

In various embodiments, the 5T4-targeting antigen-binding site or antigen-binding domain, as described herein, is further combined with an inhibitor of KRAS. Examples of KRAS inhibitors include AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C), and K-Ras(G12D)-selective inhibitory peptides, including KRpep-2 (Ac-RRCPLYISYDPVCRR-NH2) (SEQ ID NO: 243) and KRpep-2d (Ac-RRRRCPLYISYDPVCRRRR-NH2) (SEQ ID NO: 244).

In various embodiments, the 5T4-targeting antigen-binding site or antigen-binding domain, as described herein, is further combined with an inhibitor of KRAS mRNA. Illustrative KRAS mRNA inhibitors include anti-KRAS U1 adaptor, AZD-4785, siG12D-LODER™, and siG12D exosomes.

In various embodiments, the 5T4-targeting antigen-binding site or antigen-binding domain, as described herein, is further combined with an inhibitor of MEK. Illustrative MEK inhibitors that can be co-administered include binimetinib, cobimetinib, PD-0325901, pimasertib, RG-7304, selumetinib, trametinib, and selumetinib.

In various embodiments, the 5T4-targeting antigen-binding site or antigen-binding domain, as described herein, is further combined with an inhibitor of AKT. Illustrative AKT inhibitors that can be co-administered include RG7440, MK-2206, ipatasertib, afuresertib, AZD5363, and ARQ-092, capivasertib, triciribine, and ABTL-0812 (PI3K/Akt/mTOR).

In various embodiments, the 5T4-targeting antigen-binding site or antigen-binding domain, as described herein, is further combined with an inhibitor of Raf. Illustrative Raf inhibitors that can be co-administered BGB-283 (Raf/EGFR), HM-95573, LXH-254, LY-3009120, RG7304, TAK-580, dabrafenib, vemurafenib, encorafenib (LGX818), PLX8394. RAF-265 (Raf/VEGFR), and ASN-003 (Raf/PI3K).

In various embodiments, the 5T4-targeting antigen-binding site or antigen-binding domain, as described herein, is further combined with an inhibitor of ERK. Illustrative ERK inhibitors that can be co-administered include LTT-462, LY-3214996, MK-8353, ravoxertinib, GDC-0994, and ulixertinib.

In various embodiments, the 5T4-targeting antigen-binding site or antigen-binding domain, as described herein, is further combined with an inhibitor of PI3K. Illustrative PI3K inhibitors that can be co-administered include idelalisib (Zydelig®), alpelisib, buparlisib, pictilisib, eganelisib (IPI-549). Illustrative PI3K/mTOR inhibitors that can be co-administered include dactolisib, omipalisib, voxtalisib, gedatolisib, GSK2141795, and RG6114.

In various embodiments, the 5T4-targeting antigen-binding site or antigen-binding domain, as described herein, is further combined with an inhibitor of mTOR. Illustrative mTOR inhibitors that can be co-administered include as sapanisertib, vistusertib (AZD2014), ME-344, sirolimus (oral nano-amorphous formulation, cancer), and TYME-88 (mTOR/cytochrome P450 3A4).

In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov 1;408: 130-137. Also, K-RAS and mutant N-RAS can be reduced by the irreversible ERBB1/2/4 inhibitor neratinib. See, e.g., Booth, et al., Cancer Biol Ther. 2018 Feb 1;19(2): 132-137.

In various embodiments, the 5T4-targeting antigen-binding site or antigen-binding domain, as described herein, is further combined with an inhibitor of RAS. Examples of RAS inhibitors include NEO-100 and rigosertib.

In various embodiments, the 5T4-targeting antigen-binding site or antigen-binding domain, as described herein, is further combined with an antagonist of EGFR, such as AMG-595, necitumumab, ABBV-221, depatuxizumab mafodotin (ABT-414), tomuzotuximab, ABT-806, vectibix, modotuximab, and RM-1929.

In various embodiments, the 5T4-targeting antigen-binding site or antigen-binding domain, as described herein, is further combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, SAR442720, and those described in WO2018172984 and WO2017211303.

In various embodiments, the 5T4-targeting antigen-binding site or antigen-binding domain, as described herein, is further combined with an inhibitor of mitogen-activated protein kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, CK-127, cobimetinib (GDC-0973, XL-518), MT-144, selumetinib (AZD6244), sorafenib, trametinib (GSK1120212), uprosertib + trametinib, PD-0325901, pimasertib, LTT462, AS703988, CC-90003, refametinib, TAK-733, CI-1040, and RG7421.

In various embodiments, the 5T4-targeting antigen-binding site or antigen-binding domain, as described herein, is further combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, dactolisib, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, GSK2141795, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117,MLN-1117,TAK-117), SF1126, sonolisib (PX-866), RG6114, RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Spleen Tyrosine Kinase (SYK) Inhibitors

In various embodiments, the 5T4-targeting antigen-binding site or antigen-binding domain, as described herein, is further combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in US 8450321 (Gilead Connecticut) and those described in U.S. 2015/0175616.

### Tyrosine-Kinase Inhibitors (TKIs)

In various embodiments, the 5T4-targeting antigen-binding site or antigen-binding domain, as described herein, is further combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, axitinib, afatinib, ARQ-087 (derazantinib), asp5878, AZD3759, AZD4547, bosutinib, brigatinib, cabozantinib, cediranib, crenolanib, crizotinib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, olmutinib, osimertinib (AZD-9291), pazopanib, ponatinib, poziotinib, quizartinib, radotinib, rociletinib, sulfatinib (HMPL-012), sunitinib, famitinib L-malate, (MAC-4), tivoanib, TH-4000, MEDI-575 (anti-PDGFR antibody), and TAK-659.

Proteins of the present disclosure can also be used as an adjunct to surgical removal of the primary lesion.

The amount of protein and additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein may be administered during a time when the additional therapeutic agent(s) exerts its prophylactic or therapeutic effect, or vice versa.

### IV. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington’s Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein described herein, which contains a 5T4-binding site described herein, and a pharmaceutically acceptable carrier.

In certain embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:9, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:10.

The composition can be formulated for use in a variety of drug delivery systems. One or more physiologically acceptable excipients or carriers can be included in the composition for proper formulation. Suitable formulations for use in the present disclosure are found in Remington’s Pharmaceutical Sciences, Mack Publishing Company, Philadelphia, Pa., 17th ed., 1985; and Steven Shire, “Monoclonal Antibodies: Meeting the Challenges in Manufacturing, Formulation, Delivery and Stability of Final Drug Product,” Woodhead Publishing; 1st edition (Apr. 24, 2015). For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer’s solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH’s are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g. 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/ml of disodium phosphate dihydrate, 0.7 to 1.1 mg/mL of sodium dihydrogen phosphate dihydrate, and 6.0 to 6.4 mg/mL of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation include an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer’s solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg - about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present disclosure may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer’s solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

Accordingly, in some embodiments, the liquid formulation of a protein or of a lyophilized protein of the present disclosure is contained in a vial. In some embodiments, provided herein are vials comprising a liquid formulation of a protein or of a lyophilized protein described herein and a buffer, excipient, stabilizer and the like. The amount of the protein, in the liquid formulation or that is lyophilized as well as any of the buffers, excipients, stabilizers and the like form can be any combination described above and below.

The specific dose can be a uniform dose for each patient, for example, 50-5000 mg of protein. Alternatively, a patient’s dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient’s dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 µg to about 100 mg per kg of body weight, such as about 0.01 µg to about 100 mg/kg of body weight, about 0.01 µg to about 50 mg/kg of body weight, about 0.01 µg to about 10 mg/kg of body weight, about 0.01 µg to about 1 mg/kg of body weight, about 0.01 µg to about 100 µg/kg of body weight, about 0.01 µg to about 50 µg/kg of body weight, about 0.01 µg to about 10 µg/kg of body weight, about 0.01 µgto about 1 µg/kg of body weight, about 0.01 µg to about 0.1 µg/kg of body weight, about 0.1 µg to about 100 mg/kg of body weight, about 0.1 µg to about 50 mg/kg of body weight, about 0.1 µg to about 10 mg/kg of body weight, about 0.1 µg to about 1 mg/kg of body weight, about 0.1 µg to about 100 µg/kg of body weight, about 0.1 µg to about 10 µg/kg of body weight, about 0.1 µg to about 1 µg/kg of body weight, about 1 µg to about 100 mg/kg of body weight, about 1 µg to about 50 mg/kg of body weight, about 1 µg to about 10 mg/kg of body weight, about 1 µg to about 1 mg/kg of body weight, about 1 µg to about 100 µg/kg of body weight, about 1 µg to about 50 µg/kg of body weight, about 1 µg to about 10 µg/kg of body weight, about 10 µg to about 100 mg/kg of body weight, about 10 µg to about 50 mg/kg of body weight, about 10 µg to about 10 mg/kg of body weight, about 10 µg to about 1 mg/kg of body weight, about 10 µg to about 100 µg/kg of body weight, about 10 µg to about 50 µg/kg of body weight, about 50 µg to about 100 mg/kg of body weight, about 50 µg to about 50 mg/kg of body weight, about 50 µg to about 10 mg/kg of body weight, about 50 µg to about 1 mg/kg of body weight, about 50 µg to about 100 µg/kg of body weight, about 100 µg to about 100 mg/kg of body weight, about 100 µg to about 50 mg/kg of body weight, about 100 µg to about 10 mg/kg of body weight, about 100 µg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present disclosure could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the present disclosure. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1. Generation and Characterization of 5T4 Binding mAbs

This Example describes newly identified binders of 5T4 from an antibody discovery campaign. One binder, AB1002-scFv, was chosen for further development after using yeast display technology, multiple rounds of affinity maturation (CDRH3 focused and CDRH1/CDRH2 focused) for one 5T4-binder, and sequence liability analysis and correction as needed for multiple binders.

### 5T4-Antibody Generation

5T4-specific antibodies were selected starting with 131 hybridoma antibodies that bind to recombinant human 5T4-His. Of these, 113 were found to bind to human 5T4 (h5T4) on the cell surface. Of these, 78 clones showed binding to human 5T4 by surface plasmon resonance (SPR). 62 clones showed binding to rhesus 5T4 (r5T4) by SPR. 15 clones bound to h5T4 & r5T4 equally well and satisfied affinity criteria. These studies identified murine 10F10 as a binder displaying properties appropriate for a biologics drug candidate. Additional murine binders 11F09 and 08E06 were also identified as having desirable characteristics, and murine 05H04 was identified as having a subset of desirable characteristics (though lacking in binding to cynomolgus 5T4).

### Affinity Maturation of Clone 08E06

To search for variants of clone 08E06 with improved binding affinities, a yeast display affinity maturation library was created by mutating the CDRH3 residues (GGYLWFAY (SEQ ID NO:55). To enrich for scFvs that have higher affinity towards human 5T4 (h5T4), two rounds of selection were carried out with biotinylated h5T4-R-hFc-His at 1 nM. Affinities between the parental clone 08E06 and representative individual library clones were compared, and multiple rounds of FACS were performed.

Outcomes from the CDRH3 focused affinity maturation studies demonstrated an improvement in affinity, and further improvement was highly desirable. Thus, the CDRH1 and CDRH2 sequences were selected for affinity maturation (CDRH1: GYTFTSY (SEQ ID NO:53) and CDRH2: DSSDSK (SEQ ID NO:54)) using the matured CDRH3 backbone. The goal was to engineer and select binders with improved affinity over the parental clone (08E06 scFv) or the CDRH3 optimized variants described above. This created a library with a randomized CDRH1 and CDRH2 while retaining an optimized CDRH3. Multiple rounds of FACS were performed to enrich for high affinity binders of h5T4. 53 affinity matured clones were obtained in total from these processes. CDR sequences of select resultant affinity matured variants of clone 08E06 are shown in Table 5.

TABLE 5

Affinity Matured Variants of clone 08E06

| | | | |
|---|---|---|---|
| Clone | CDRH1 | CDRH2 | CDRH3 |
| 08E06 | GYTFTSY (SEQ ID NO:53) | DSSDSK (SEQ ID NO:54) | GGYLWFAY (SEQ ID NO:55) |
| AB1558 | GYTFGSY (SEQ ID NO:73) | DASTEK (SEQ ID NO:74) | GGYLWFQY (SEQ ID NO:75) |
| AB1566 | GYLFTSY (SEQ ID NO: 76) | SVSDAK (SEQ ID NO:77) | GGYLWFKY (SEQ ID NO:78) |
| AB1554 | GYTFGSY (SEQ ID NO:73) | DARSAK (SEQ ID NO: 79) | GGYLWFKY(SEQ ID NO: 78) |
| AB1574 | GYRFTSY (SEQ ID NO: 80) | DASSAK (SEQ ID NO:81) | GGYLWFKY (SEQ ID NO:78) |
| AB1579 | GYGFTSY (SEQ ID NO:82) | DARTAK (SEQ ID NO:83) | GGYLWYAY (SEQ ID NO:84) |
| AB1540 | GYTFTSY (SEQ ID NO:53) | DASDAK (SEQ ID NO:85) | GGYLWYHY (SEQ ID NO:86) |
| AB1541 | GYTFTSY (SEQ ID NO:53) | DASDAK (SEQ ID NO:85) | GGYLWYSY (SEQ ID NO:87) |
| AB1552 | GYTFTSY (SEQ ID NO:53) | DASDAK (SEQ ID NO:85) | GGYLWYAY (SEQ ID NO:84) |
| AB1565 | GYSFTSY (SEQ ID NO:88) | DASDAK (SEQ ID NO:85) | GGYLWFKY (SEQ ID NO:78) |
| AB1534 | GYTFTSY (SEQ ID NO:53) | DASDAK (SEQ ID NO:85) | GGYLWFKY (SEQ ID NO:78) |
| AB1573 | GYGFTSY (SEQ ID NO:82) | DARTAK (SEQ ID NO:83) | GGHLWYAY (SEQ ID NO:89) |

### Humanization and Sequence Liability Assessment

Clones 10F10 and 11F09 were humanized into multiple framework sequences; the sequences of these humanized clones are provided in Table 1 above. Because these clones contained amino acids in their CDRs that could negatively impact protein expression, stability, or immunogenicity, clones were designed with substitutions at these amino acids. Sequences of these liability-corrected clones are also provided in Table 1, above.

AB 1002 (a humanized variant of murine 10F10, with VH T62S correction to replace rare residue T62), was ultimately selected for further development.

### Epitope Mapping

Binding of 5T4 binders in relation to reference 5T4 antibodies was performed to determine binding epitope. The epitopes of murine 10F10 and murine 11F09 were mapped onto 5T4 in the leucine-rich repeat 1 (LRR1) domain. Furthermore, the epitope of murine 08E06 was mapped onto 5T4 in the leucine-rich repeat 2 (LRR2) domain. See, e.g., Zhao, et al., Structure (2014) 22(4):612-20.

### Surface Plasmon Resonance (SPR)

AB1002 scFv (VL-VH) (a humanized variant of 10F10) was converted into a multispecific binding protein comprising the 5T4-scFv, and two non-5T4 binders, to yield AB1310/AB1783. Additionally, 08E06 humanized variant (AB0063 (VH-VL) and AB0064 (VL-VH)) was converted similarly to two multispecific binding proteins. The binding affinities of AB1310/AB1783 to 5T4 were measured by surface SPR. Briefly, SPR was performed using a Biacore 8 K instrument at physiological temperature of 37° C. Briefly, human Fc specific antibodies were covalently immobilized at a density of about 8000-10000 resonance units (RU) on carboxy methyl dextran matrix of a CM5 biosensor chip to create an anti-hFc IgG chip. Samples were injected on the anti-hFc IgG chip at a flow rate of 5-10 µL/min for 60 seconds. Protein was serially diluted (300 nM-0.14 nM) in three-fold dilutions with running buffer and injected at a flow rate of 30 µl/min over the captured test articles. Association was monitored for 240-300 seconds, and dissociation was monitored for 300-900 seconds. Surfaces were regenerated between cycles with three pulses of 10 mM glycine-HCl (pH 1.7) injected for 20 seconds at 100 µL/min.

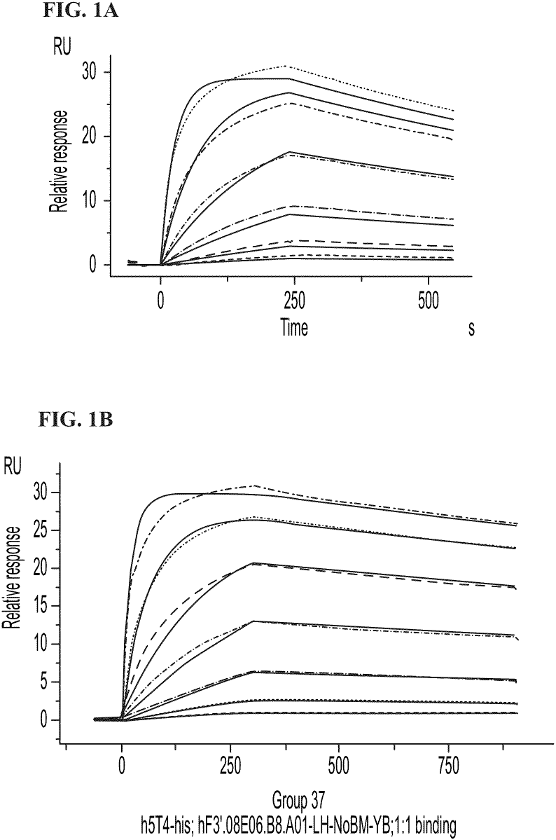

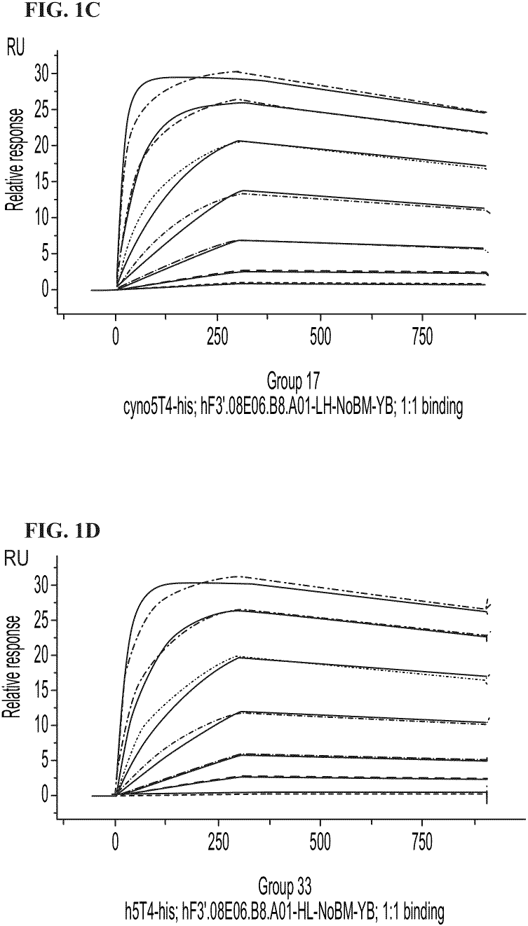

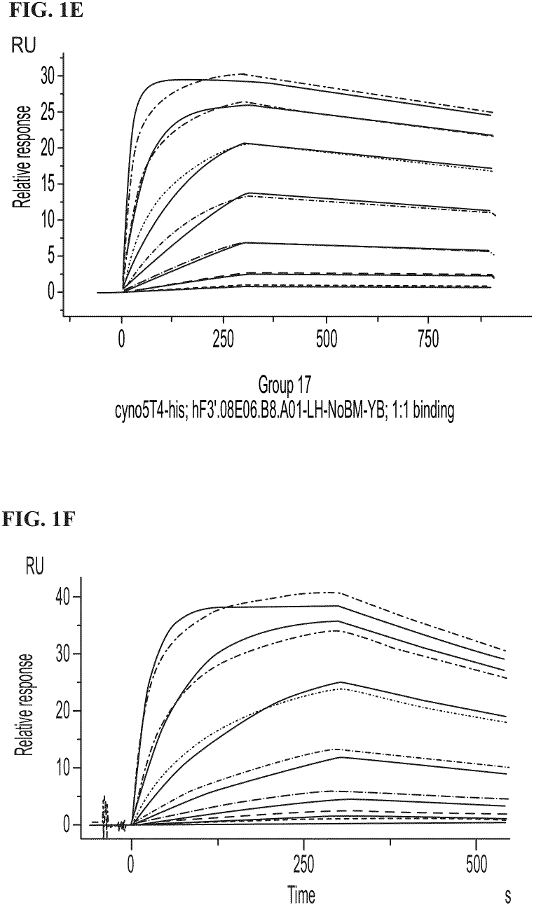

SPR analysis showed that AB1310/AB1783, AB0063, and AB0064 had high affinity for h5T4 (FIGS. 1A-1E). This binding affinity was maintained at a lower pH (6.0), which is representative of the tumor microenvironment (FIG. 1F). Details of SPR analysis of AB1310/AB1783 binding to 5T4 are shown in Table 6, and comparative binding to human and cynomolgus monkey 5T4 for AB1310 is shown in Table 7.

TABLE 6

Binding affinity of AB1310/AB1783, AB0063, and AB0064 in SPR assay

| Molecule | pH | kₐ (M⁻¹s⁻¹) | k_{d} (s⁻¹) | Kinetics fit K_{D} (nM) |
|---|---|---|---|---|
| AB1310/AB1783 | 7.4 | (1.3 ± 0.0) × 10⁵ | (7.7 ± 0.3) × 10⁻⁴ | 6.1 ± 0.2 |
| | 6.0 | 5 (1.2 ± 0.1) × 10⁵ | -3 (1.2 ± 0.0) × 10⁻³ | 9.4 ± 0.6 |
| AB0064 | 7.4 | 1.56 × 10⁵ | 2.49 × 10⁻⁴ | 1.6 |
| AB0063 | 7.4 | 1.28 × 10⁵ | 2.42 × 10⁻⁴ | 1.9 |

TABLE 7

Binding affinity of AB 1310 in SPR assay

| Species | K_{D} 5T4 (nM) |
|---|---|
| Human | 6.2 ± 0.4 |
| Cynomolgus monkey | 8.3 ± 2.2 |

Studies were conducted to assess the binding affinity of the 5T4-targeting TriNKET® to 5T4 expressed on the cell surface. The KYSE-30 and H292 human cancer cell lines, derived from esophageal squamous cell carcinoma and non-small cell lung carcinoma, respectively, were used. KYSE-30 cells express higher levels of surface 5T4 than is found on H292 cells. Briefly, tumor cells were incubated with 5T4-TriNKET® or 5T4-mAb at 4° C. for 1 hour. After incubation, binding patterns of the TriNKET® and mAb to 5T4+ cells were detected using a fluorophore conjugated anti-human IgG secondary antibody. Cells were analyzed by flow cytometry and fold MFI over secondary-only controls reported. Additionally, the same protocol was used to assess binding of 5T4-TriNKET® to 5T4-expressing primary lung cancer-associated fibroblasts.

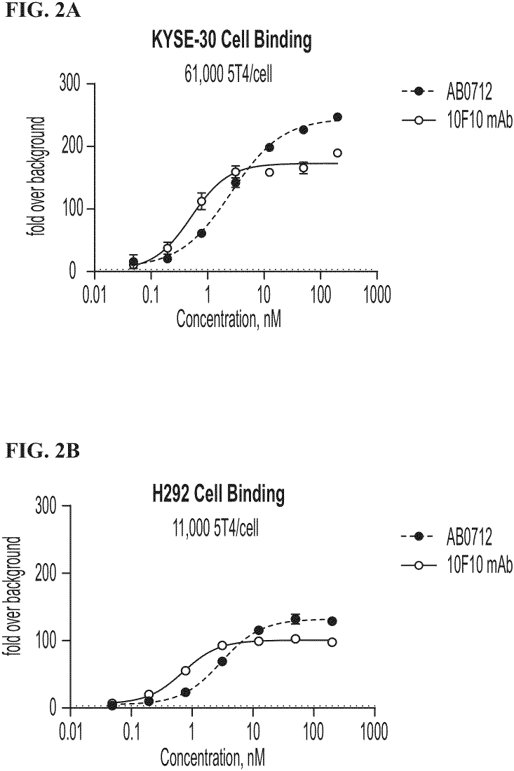

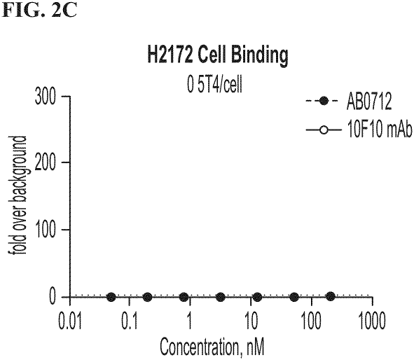

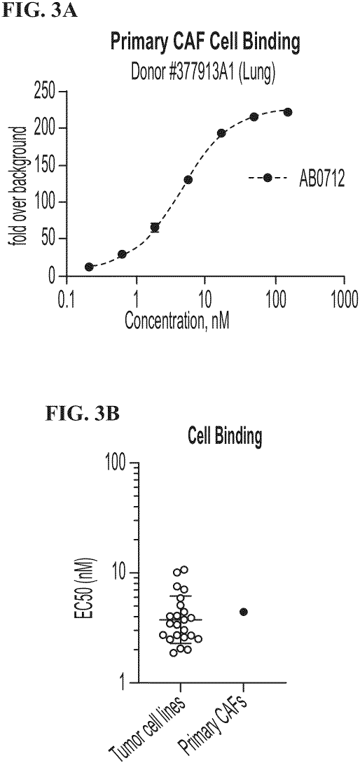

FIG. 2A, FIG. 2B, FIG. 3A and FIG. 3B show binding to 5T4-positive target cells after incubation with 5T4-TriNKET® or 5T4-mAb. TriNKET® bound to a higher magnitude on KYSE-30 cells, which express greater levels of surface 5T4 than H292 cells do. The 5T4-targeting TriNKET® bound the cells with single-digit nM concentrations and with higher maximum binding than 5T4-mAb. In addition, robust binding of 5T4-TriNKET® was observed to primary cancer-associated fibroblasts. By contrast, neither AB1310/AB1783-TriNKET® nor the parental monoclonal antibody (10F10), showed binding to the 5T4⁻ cell line H2712 (FIG. 2C).

### Assessment of Specificity to h5T4

A flow cytometry based polyspecificity reagent (PSR) assay allows the filtering out of antibodies that have a higher probability to bind non-specifically to unrelated proteins. PSR assays correlate well with cross-interaction chromatography, a surrogate for antibody solubility, as well as with baculovirus particle enzyme-linked immunosorbent assay, a surrogate for in vivo clearance (Xu et. al (2013). Addressing polyspecificity of antibodies selected from an in vitro yeast presentation system: a FACS-based, high-throughput selection and analytical tool. Protein engineering design and selection, 26, 663-670).

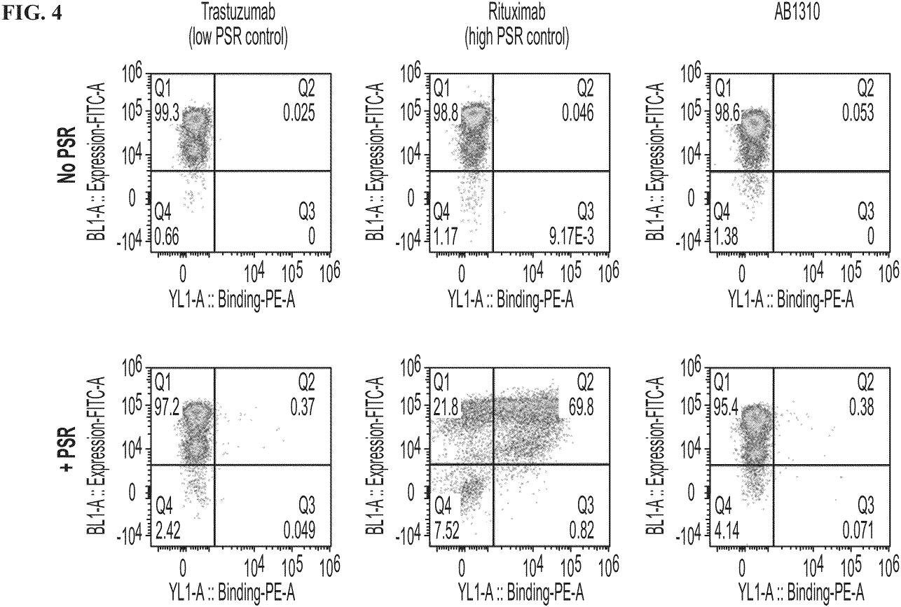

50 µL of 100 nM TriNKET® or control mAb in PBSF were incubated with prewashed 5 µL protein A dyna beads slurry (Invitrogen, catalog # 10001D) for 30 minutes at room temperature. TriNKET® or mAb bound magnetic beads were allowed to stand on a magnetic rack for 60 seconds and the supernatant was discarded. The bound beads were washed with 100 µL PBSF. Beads were incubated for 20 minutes on ice with 50 µL of biotinylated PSR reagent which was diluted 25-fold from the stock (Xu et. al., (2013) Protein engineering design and selection, 26, 663-670). Samples were put on the magnetic rack, supernatant discarded, and washed with 100 µL of PBSF. A secondary FACS reagent, to detect binding of biotinylated PSR reagent to TriNKET®s or control mAbs, was made as follows: 1:250 µL of Streptavidin-PE (Biologend, catalog # 405204) and 1:100 donkey anti-human Fc were combined in PBSF. To each sample, 100 µL of the secondary reagents were added and allowed to incubate for 20 minutes on ice. The beads were washed twice with 100 µL PBSF, and samples were analyzed on a FACS Celesta (BD). AB1310/AB1783 was shown to lack non-specific interactions by the PSR assay (FIG. 4).

### Assessment of Surface Binding to h5T4 in Vitro

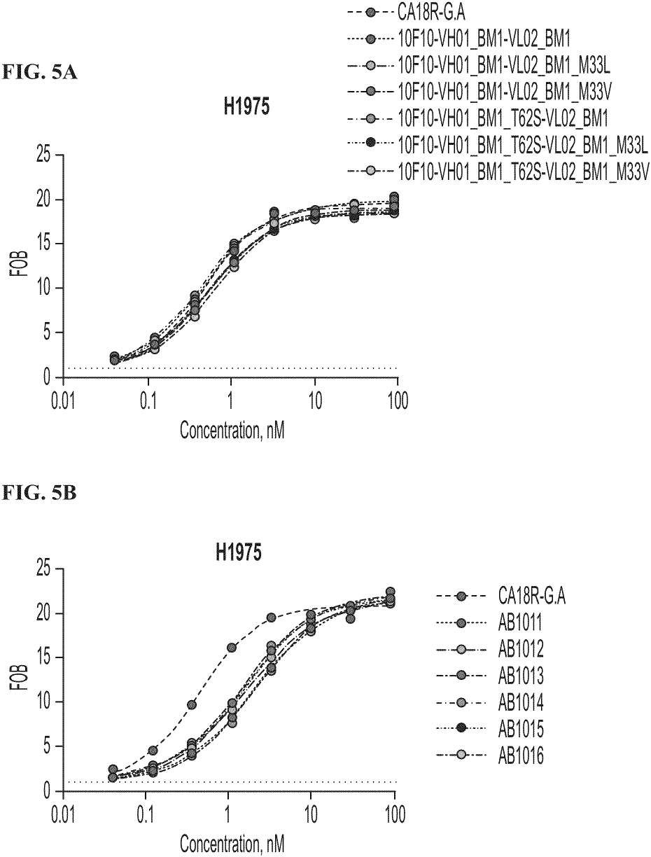

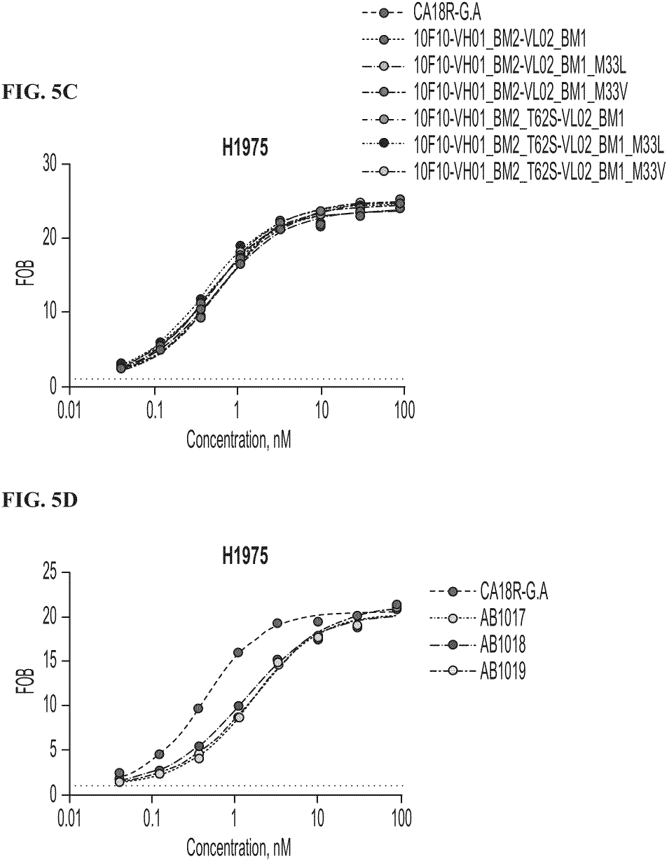

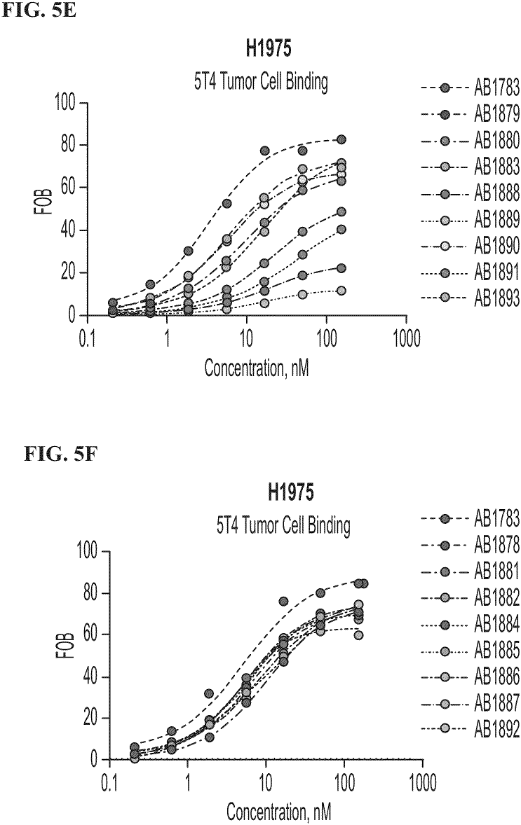

Identified 5T4-binding clones 10F10 and 11F09 were both shown to maintain binding to 5T4⁺ tumor cells H1975 following humanization and sequence liability alteration (FIGS. 5A-5D) using the binding affinity assay described above. FIG. 5E and FIG. 5F are graphs showing binding (fold over background (FOB)) of various concentrations of humanized 5T4 binders.

Further, AB 1310/AB1783 bound with single-digit nanomolar relative affinity (1.5 - 7.6 nM EC50 values) on a panel of tumor cell lines representing a range of 5T4 expression, and did not bind a 5T4 knockout line, demonstrating high affinity and specificity, shown in Table 8.

TABLE 8

Binding of AB1310/AB1783 to 5T4-expressing tumor cell lines

| Target Cell line | Origin | 5T4 per cell | Cell binding EC50 (nM) |
|---|---|---|---|
| KYSE-30 | HNSCC | 61,000 | 2.5 |
| HCC4006 | NSCLC-adeno | 53,000 | 7.6 |
| FaDu | HNSCC | 22,000 | 7.1 |
| H1975 | NSCLC - adeno | 22,000 | 1.9 |
| Detroit 562 | HNSCC | 21,000 | 2.7 |
| BT474 | BRC | 20,000 | 3.7 |
| 786-O | RCC | 18,000 | 2.7 |
| H1703 | NSCLC - SCC | 15,000 | 2.6 |
| HCT116 | CRC | 14,000 | 2.1 |
| H292 | NSCLC | 12,000 | 1.5 |
| HT29 | CRC | 5,000 | 4.1 |
| H2172 | NSCLC | 0 | N/A |
| H292-5T4KO | NSCLC | 0 | N/A |

In conclusion, an antibody discovery campaign for 5T4 binders was conducted. Multiple 5T4 binders, including 10F10, 11F09, 05H04, and 08E06, were identified as promising candidates for further development. AB1002 (a humanized variant of murine 10F10, with VH T62S correction to replace rare residue T62), particularly, displayed desirable properties of a biologic.

### APPENDIX

### Exemplary 5T4-Binding Site Sequences

### AB2029 - hF3′.5T4

### scFv-Fc

ENVLTQSPATLSLSPGERATLSCSAKSSVSYIHWYQQKPGQAPRLLIYDT
SYLGSGIPARESGSGSGTDYTLTISSLEPEDFAVYYCQQWSSYPYTFGCG
TKVEIKGGGGSGGGGSGGGGSGGGGSQVQLVQSGAEVKKPGASVKVSCKA
SGYKFTDYYMDWVRQAPGQCLEWIGYIFPNDASTTYNEKFKGKATLTADK
STNTAYMELSSLRSEDTAVYYCARSRDADYWGQGTTVTVSSGSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPRVYTLPPCRDELTKNQVSLTCLVKGFYPSDI
AVEWESNGQPENNYKTTPPVLVSDGSFTLYSKLTVDKSRWQQGNVFSCSV
MHEALHNHYTQKSLSLSPG (SEQ ID NO:140)

### AB2093 - hF3′.5T4

### scFv-Fc

QVQLVQSGAEVKKPGASVKVSCKASGYKFTDYYMDWVRQAPGQCLEWIGY
IFPNDASTTYNEKFKGKATLTADKSTNTAYMELSSLRSEDTAVYYCARSR
DADYWGQGTTVTVSSGGGGSGGGGSGGGGSGGGGSENVLTQSPATLSLSP
GERATLSCSAKSSVSYIHWYQQKPGQAPRLLIYDTSYLGSGIPARFSGSG
SGTDYTLTISSLEPEDFAVYYCQQWSSYPYTFGCGTKVEIKGSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPRVYTLPPCRDELTKNQVSLTCLVKGFYPSDI
AVEWESNGQPENNYKTTPPVLVSDGSFTLYSKLTVDKSRWQQGNVFSCSV
MHEALHNHYTQKSLSLSPG (SEQ ID NO:141)

### AB2143 - hF3′.5T4

### scFv-Fc

EVQLVQSGAEVKKPGASVKVSCKASGYKFTDYYMDWVRQAPGQCLEWIGY
IFPNDASTTYNEKFKGKATLTADKSTNTAYMELSSLRSEDTAVYYCARSR
DADYWGQGTTVTVSSGGGGSGGGGSGGGGSGGGGSENVLTQSPATLSLSP
GERATLSCSAKSSVSYIHWYQQKPGQAPRLLIYDTSYLGSGIPARFSGSG
SGTDYTLTISSLEPEDFAVYYCQQWSSYPYTFGCGTKVEIKGSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPRVYTLPPCRDELTKNQVSLTCLVKGFYPSDI
AVEWESNGQPENNYKTTPPVLVSDGSFTLYSKLTVDKSRWQQGNVFSCSV
MHEALHNHYTQKSLSLSPG (SEQ ID NO:142)

### AB2509 — hF3.5T4 (comprises a Fab Binding 5T4)

### Chain L:

ENVLTQSPATLSLSPGERATLSCSAKSSVSYIHWYQQKPGQAPRLLIYDT
SYLGSGIPARESGSGSGTDYTLTISSLEPEDFAVYYCQQWSSYPYTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC (SEQ ID NO:143)

### Chain H:

QVQLVQSGAEVKKPGASVKVSCKASGYKFTDYYMDWVRQAPGQGLEWIGY
IFPNDASTTYNEKFKGKATLTADKSTNTAYMELSSLRSEDTAVYYCARSR
DADYWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVCTLP
PSRDELTENQVSLTCLVKGFYPSDIAVEWESNGQPENNYKTTPPVLDSDG
SFFLYSWLTVDKSRWQQGNVFSCSVMHEALHNHYTQKSLSLSPG (SEQ
ID NO:144)

### AB2511 — hF3.5T4 (Comprises a Fab Binding 5T4)

### Chain L

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC (SEQ ID NO:145)

### Chain H

EVQLVESGGGLVKPGGSLRLSCEASGFTFSDYGMHWVRQAPGKGLEWLAY
ISSGSSTFYYADTVKGRFTISRDNAKNSLYLQMNSLRAEDTAVYYCGSSQ
SYYRGTMDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
(SEQ ID NO:146)

### AB2512 — hF3.5T4 (Comprises a Fab Binding 5T4)

### Chain L

DIQLTQSPSSLSASVGDRVTITCRASQGVSTSTYTYLHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC (SEQ ID NO:147)

### Chain H

EVQLVESGGGLVKPGGSLRLSCEASGFTFSDYGMHWVRQAPGKGLEWLAY
ISSGSSTFYYADTVKGRFTISRDNAKNSLYLQMNSLRAEDTAVYYCGSSQ
SYYRGTMDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VCTLPPSRDELTENQVSLTCLVKGFYPSDIAVEWESNGQPENNYKTTPPV
LDSDGSFFLYSWLTVDKSRWQQGNVFSCSVMHEALHNHYTQKSLSLSPG
(SEQ ID NO:148)

### AB2513 — hF3.5T4 (comprises a Fab Binding 5T4)

### Chain L

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASWCLLNNFYPREAKVQ
WKVDNALQSGNSQESVTEQDSKDSTYSLSSTLTLSKADYEKHKVYACEVT
HQGLSSPVTKSFNRGEC (SEQ ID NO:149)

### Chain H

EVQLVESGGGLVKPGGSLRLSCAASRFTFSDFGMHWVRQAPGKGLEWVSY
ISSGSSTIYYADTVKGRFTISRDNAKNSLYLQMNSLRAEDTAVYYCASSQ
SYYRGTLDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVC
TLPPSRDELTENQVSLTCLVKGFYPSDIAVEWESNGQPENNYKTTPPVLD
SDGSFFLYSWLTVDKSRWQQGNVFSCSVMHEALHNHYTQKSLSLSPG (S
EQ ID NO:150)

### AB2514 — hF3.5T4 (comprises a Fab Binding 5T4)

### Chain L

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC (SEQ ID NO:151)

### Chain H

EVQLVESGGGLVKPGGSLRLSCAASRFTFSDFGMHWVRQAPGKGLEWVSY
ISSGSSTIYYADSVKGRFTISRDNAKNSLYLQMNSLRAEDTAVYYCASSQ
SYYRGTLDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVWDVSHEDPEVKFNWYVDGVEVHNAKTKPREEQYNS
TYRWSVLTVLHQDWLNGKEYKCKVSNKALPAPIEKTISKAKGQPREPQVC
TLPPSRDELTENQVSLTCLVKGFYPSDIAVEWESNGQPENNYKTTPPVLD
SDGSFFLYSWLTVDKSRWQQGNVFSCSVMHEALHNHYTQKSLSLSPG (S
EQ ID NO:152)

### AB1878 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASRFTFSDFGMHWVRQAPGKCLEWVSYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:153)

### AB1881 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASRFTFSDFGMHWVRQAPGKCLEWIAYISSGSSTIYYADTVKGRFT
ISRDNAKNTLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:154)

### AB1882 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASRFTFSDFGMHWVRQAPGKCLEWVSYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:155)

### AB1884 - hF3′ (alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASRFTFSDFGMHWVRQAPGKCLEWIAYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:156)

### AB1885 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSQVQLVESGGGLVKPGGSLR
LSCAASRFTFSDFGMHWVRQAPGKCLEWIAYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:157)

### AB1886 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASRFTFSDFGMHWIRQAPGKCLEWVSYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:158)

### AB1887 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASRFTFSDFGMHWVRQAPGKCLEWIAYISSGSSTIYYADTVKGRFT
ISRDNAKNSLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:159)

### AB1892 - hF3′ (Alternative Humanization of Clone 10F10)

### scFv-Fc

DIQLTQSPSSLSASVGDRVTITCRASQSVTTSNYNYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASRFTFSDFGMHWVRQAPGKCLEWVSYISSGSSTIYYADTVKGRFT
ISRDNSKNTLYLQMNSLRAEDTAVYYCASSQSYYRGTLDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:160)

### AB1319 - hF3′

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCEASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SASDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:161)

### AB1879 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASGFTFSDYGMHWVRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:162)

### AB1880 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCEASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:163)

### AB1883 - hF3′ (alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSQVQLVESGGGLVKPGGSLR
LSCAASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:164)

### AB1888 - hF3′ (alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASGFTFSDYGMHWVRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:165)

### AB1889 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVQPGGSLR
LSCAASGFTFSDYGMHWVRQAPGKCLEWLAYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:166)

### AB1890 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASGFTFSDYGMHWVRQAPGKCLEWVSYISSGSSTFYYADTVKGRFT
ISRDNSKNTLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:167)

### AB1891 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLVESGGGLVKPGGSLR
LSCAASGFTFSDYGMHWIRQAPGKCLEWVSYISSGSSTFYYADTVKGRFT
ISRDNAKNSLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:168)

### AB1893 - hF3′ (Alternative Humanization of Clone 11F09)

### scFv-Fc

DIQLTQSPSSLSASVGDRVTITCRASQGVSTSTYTYMHWFQQKPGKAPKL
LIKFASNLESGVPSRFSGSGSGTDFTLTISSLQPEDFATYYCQHSWEIPW
TFGCGTKVEIKGGGGSGGGGSGGGGSGGGGSEVQLLESGGGLVQPGGSLR
LSCAASGFTFSDYGMHWVRQAPGKCLEWLAYISSGSSTFYYADTVKGRFT
ISRDNAKNTLYLQMNSLRAEDTAVYYCGSSQSYYRGTMDYWGQGTTVTVS
SGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPRVYTLPPCRDELTKNQVSLTC
LVKGFYPSDIAVEWESNGQPENNYKTTPPVLVSDGSFTLYSKLTVDKSRW
QQGNVFSCSVMHEALHNHYTQKSLSLSPG (SEQ ID NO:169)

### INCORPORATION BY REFERENCE

Unless stated to the contrary, the entire disclosure of each of the patent documents and scientific articles referred to herein is incorporated by reference for all purposes. Sequences listed in the accompanying Appendix filed concurrently with this application are incorporated by reference as part of the disclosure described herein for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1. An antigen-binding site that binds 5T4, comprising:
a heavy chain variable domain (VH) comprising a complementarity-determining region 1 (CDR1) sequence comprising SEQ ID NO:3, a complementarity-determining region 2 (CDR2) sequence comprising SEQ ID NO:4, and a complementarity-determining region 3 (CDR3) sequence comprising SEQ ID NO:5; and
a light chain variable domain (VL) comprising a CDR1 sequence comprising SEQ ID NO:6, a CDR2 sequence comprising SEQ ID NO:7, and a CDR3 sequence comprising SEQ ID NO:8.

2-5. (canceled)

6. The antigen-binding site of claim 1, wherein:
a. the VH comprises an amino acid sequence of SEQ ID NO:9 and the VL comprises an amino acid sequence of SEQ ID NO: 10,
b. the VH comprises an amino acid sequence of SEQ ID NO: 11 and the VL comprises an amino acid sequence of SEQ ID NO: 12,
c. the VH comprises an amino acid sequence of SEQ ID NO:22 and the VL comprises an amino acid sequence of SEQ ID NO: 10,
d. the VH comprises an amino acid sequence of SEQ ID NO:24 and the VL comprises an amino acid sequence of SEQ ID NO: 10,
e. the VH comprises an amino acid sequence of SEQ ID NO:26 and the VL comprises an amino acid sequence of SEQ ID NO: 10,
f. the VH comprises an amino acid sequence of SEQ ID NO: 108 and the VL comprises an amino acid sequence of SEQ ID NO: 10,
g. the VH comprises an amino acid sequence of SEQ ID NO: 138 and the VL comprises an amino acid sequence of SEQ ID NO: 10,
h. the VH comprises an amino acid sequence of SEQ ID NO:28 and the VL comprises an amino acid sequence of SEQ ID NO: 10,
i. the VH comprises an amino acid sequence of SEQ ID NO:30 and the VL comprises an amino acid sequence of SEQ ID NO: 10, or
j. the VH comprises an amino acid sequence of SEQ ID NO: 1 and the VL comprises an amino acid sequence of SEQ ID NO:2.

7. The antigen-binding site of claim 1, wherein the VH comprises an amino acid sequence at least 90%, at least 91%, at least 92%, at least 93%, at least 94%, at least 95%, at least 96%, at least 97%, at least 98%, or at least 99% identical to the amino acid sequence of SEQ ID NO:9.

8. The antigen-binding site of claim 1, wherein the VH comprises a G44C substitution relative to SEQ ID NO:9, wherein the numbering is according to the Kabat numbering scheme.

9-10. (canceled)

11. The antigen-binding site of claim 1, wherein the VL comprises an amino acid sequence at least 90%, at least 91%, at least 92%, at least 93%, at least 94%, at least 95%, at least 96%, at least 97%, at least 98%, or at least 99% identical to the amino acid sequence of SEQ ID NO: 10.

12-13. (canceled)

14. The antigen-binding site of any claim 1, wherein the VL comprises the amino acid sequence of SEQ ID NO: 10.

15. An antigen-binding site comprising a VH comprising the amino acid sequence of SEQ ID NO:94 and a VL comprising the amino acid sequence of SEQ ID NO: 10.

16. An antigen-binding site comprising a VH comprising at least 95% identity to the amino acid sequence of SEQ ID NO:9 and a VL comprising at least 95% identity to the amino acid sequence of SEQ ID NO: 10, or a VH comprising at least 95% identity to the amino acid sequence of SEQ ID NO: 11 and a VL comprising at least 95% identity to the amino acid sequence of SEQ ID NO: 12.

17-27. (canceled)

28. The antigen-binding site of claim 1, wherein the antigen-binding site is present as a single-chain fragment variable (scFv), a Fab fragment, or a monoclonal antibody.

29. The antigen-binding site of claim 1, wherein the antigen-binding site is present as a single-chain fragment variable (scFv).

30. The antigen-binding site of claim 29, wherein the scFv comprises a sequence selected from the group consisting of SEQ ID NO:95 and SEQ ID NO:96.

31. The antigen-binding site of claim 29, wherein the scFv comprises a sequence selected from the group consisting of SEQ ID NO: 13 and SEQ ID NO: 14.

32-33. (canceled)

34. A protein comprising the antigen-binding site of claim 1.

35-43. (canceled)

44. An isolated nucleic acid molecule encoding one or more of the VH or VL comprising the antigen-binding site of claim 1.

45-48. (canceled)

49. A bispecific T-cell engager comprising the antigen-binding site of claim 1 and an antigen-binding site that binds CD3.

50-61. (canceled)

62. One or more isolated nucleic acid molecules encoding:
a. a VH comprising a CDR1 sequence comprising SEQ ID NO:3, a CDR2 sequence comprising SEQ ID NO:4, and a CDR3 sequence comprising SEQ ID NO:5; and/or
b. a VL comprising a CDR1 sequence comprising SEQ ID NO:6, a CDR2 sequence comprising SEQ ID NO:7, and a CDR3 sequence comprising SEQ ID NO:8.

63. One or more isolated nucleic acid molecules encoding:
a. a VH comprising the amino acid sequence of SEQ ID NO:9; and
b. a VL comprising the amino acid sequence of SEQ ID NO: 10.

64. One or more isolated nucleic acid molecules encoding:
a. a VH comprising the amino acid sequence of SEQ ID NO: 11; and
b. a VL comprising the amino acid sequence of SEQ ID NO: 12.

65-70. (canceled)

71. A pharmaceutical composition comprising the protein of claim 34; and a pharmaceutically acceptable carrier.

72. A method of treating cancer, the method comprising administering to a subject in need thereof an effective amount of the protein of claim 34.

73-80. (canceled)

81. A method of enhancing tumor cell death, the method comprising exposing the tumor cell to an effective amount of antigen the protein of claim 34.

82-83. (canceled)
